(19) United States
(12) Patent Application Publication
(10) Pub. No.: US 2026/0171206 A1
(43) Pub. Date: Jun. 18, 2026

(54) SYSTEMS AND METHODS FOR SUBJECT, DEVICE, AND MEDICAMENT VERIFICATION

(71) Applicant: Rocket Science Health Corp., Victoria (CA)

(72) Inventors: Kenneth IRVING, Victoria (CA); Jason DELICHTE, Victoria (CA); Darren DELICHTE, Victoria (CA); James JACKSON, Victoria (CA); Lyndon SOBOLIK, Victoria (CA); Aaron GLENN, Victoria (CA)

(21) Appl. No.: 19/531,909

(22) Filed: Feb. 6, 2026

Related U.S. Application Data
(63) Continuation of application No. PCT/IB2024/057630, filed on Aug. 6, 2024.
(60) Provisional application No. 63/518,054, filed on Aug. 7, 2023.
(60) Provisional application No. 63/555,621, filed on Feb. 20, 2024.

(51) Int. Cl. G16H 20/10; G16H 30/40

(52) U.S. Cl. CPC G16H 20/10; G16H 30/40

(57) ABSTRACT

Disclosed herein are systems, devices and methods for delivering a composition to a subject. The method may include capturing images of the subject, identifying the subject based on images and other criteria, capturing images of a device, the device comprising a marker, the device transitioning between a plurality of configuration states, identifying a configuration state based on the images of the device, wherein the marker is partially concealed in a first configuration state, and providing feedback based on identifying the device, configuration state, and/or the subject.

### CROSS REFERENCE

This application is a continuation of International Application No. PCT/IB2024/057630, filed Aug. 6, 2024, which claims the benefit to U.S. Provisional Application No. 63/518,054, filed Aug. 7, 2023, and U.S. Provisional Application No. 63/555,621, filed Feb. 20, 2024, the contents of which are incorporated herein by reference.

### BACKGROUND

It is often desirable to verify the identity of a subject in connection with the administration of medication, or other procedures which are based upon a physical sample collected from a subject (e.g., alcohol breath analysis). Presently, administration of many controlled medications does not occur in a manner which verifies the identity of the recipient which leads to diversion of the controlled medication, and other procedures which are based upon a physical sample collected from a subject (e.g., alcohol breath analysis) must be completed in person. It is also desirable to ensure that the subject has correctly administered a medication.

### SUMMARY

It is appreciated by the inventors that methods and systems for verifying a subject's identity in connection with administration of medication, or collection of a physical sample would offer significant benefits to the compliance and safety, ensuring the authenticity and reliability of the procedures, and that there is presently a lack of available methods and systems for accomplishing the same. In addition, verification of adherence to programs for clinical trials, treatment programs, or similar processes which require a patient to follow a prescribed dosage schedule is desirable, for example, verifying administration, verifying administration of the correct doses at the proper times, or correct dosing protocol, can improve resulting data. As such, provided herein are methods and systems for verifying a subject for administration of a medicament to the subject.

An aspect of the embodiments herein disclose a method for delivery of a composition to a target region of a subject, comprising: capturing one or more images of the subject; identifying the subject based on the one or more images of the subject and one or more criteria, wherein the one or more criteria comprise the subject being in need of the composition; capturing one or more images of a device, wherein the device comprises a marker and a housing, wherein the device is configured to transition between a plurality of configuration states based on a position of the marker and the housing; identifying the device as being in a first configuration state of the plurality of configuration states based on the one or more images of the device, wherein the marker is not concealed by the housing in the first configuration state; at least one of: providing an indicator to deliver the composition to the target region based on identifying the device as being in the first configuration state and identifying the subject based on the one or more criteria; or displaying a recommendation to deliver the composition to the target region based on identifying the device as being in the proper configuration state and identifying the subject based on the one or more criteria; and verifying actuation of the device based on a change in the configuration state of the device, thereby verifying delivery of the composition of the subject. In some embodiments, the device further comprises a second housing. In some embodiments, the marker is a fiducial marker. In some embodiments, the first configuration state of the plurality of configuration states indicates an alignment of the device with respect to the target region of the subject. In some embodiments, the target region is an orifice of the subject, the mouth of the subject, the nasal cavity of the subject, an olfactory region of the subject, or the columella of the subject. In some embodiments, the marker is at least partially concealed by the housing and the housing is visible in a second configuration state of the plurality of configuration states. In some embodiments, the second housing is concealed in a second configuration state of the plurality of configuration states. In some embodiments, the marker is at least partially concealed by the housing and the housing is not visible in a third configuration state of the plurality of configuration states. In some embodiments, the second housing is visible in a third configuration state of the plurality of configuration states. In some embodiments, the marker is not concealed and the housing is concealed in a fourth configuration state of the plurality of configuration states. In some embodiments, the second housing is visible in a fourth configuration state of the plurality of configuration states. In some embodiments, the housing comprises a first housing marker. In some embodiments, the first housing marker comprises an annular ring. In some embodiments, the first housing marker comprises a color distinct from the housing. In some embodiments, the second housing comprises a second housing marker. In some embodiments, the second housing marker comprises a color distinct from the first housing marker. In some embodiments, the marker is a QR code. In some embodiments, the marker is an ArUco marker. In some embodiments, the device being in the first configuration state indicates the device can be actuated. In some embodiments, the method further comprising monitoring the device and/or the subject. In some embodiments, based on the monitoring, the method further comprises providing a second indicator, wherein the second indicator prevents the device from delivering the composition. In some embodiments, the second indicator is provided based on the subject being unidentifiable during the monitoring or the device not being in the first configuration state during the monitoring. In some embodiments, based on the monitoring, the method further comprises providing a third indicator after providing the second indicator, wherein the third indicator allows the device to deliver the composition. In some embodiments, the third indicator is provided based on the subject becoming identifiable during the monitoring or the device being in the first configuration state during the monitoring after previously being in a different configuration state. In some embodiments, the marker comprises one or more submarkers. In some embodiments, in the second configuration state, a first submarker of the one or more submarkers is concealed by the housing and a second submarker of the one or more submarkers is not concealed by the housing and the housing is not concealed. In some embodiments, in the first configuration state, the first submarker of the one or more submarkers is not concealed, and the second submarker of the one or more submarkers is not concealed, and the housing is not concealed. In some embodiments, in the third configuration state, the first submarker of the one or more submarkers is not concealed, a second submarker of the one or more submarkers is concealed, and the housing is concealed. In some embodiments, the device is partially inserted into a nasal cavity of the subject, causing the device to transition from the first configuration state to the second configuration state. In some embodiments, the device is partially inserted into the nasal cavity of the subject at a prescribed depth and angle. In some embodiments, the method further comprises determining an alignment state of the device and the subject based upon a position of the marker. In some embodiments, the method further comprises determining an alignment state of the device and the subject based upon a position of the marker relative to one or more subject facial features, the one or more criteria, or combinations thereof. In some embodiments, the position comprises the alignment state of the device, and wherein the method further comprises providing a recommendation to adjust the alignment state of the device if it is determined the alignment state is improper relative to the one or more subject facial features. In some embodiments, the identifying the subject comprises analyzing an image amongst the one or more images, wherein the image is of the subject's face, and associating at least 68 data points to facial features of the subject. In some embodiments, the data points associated with facial features of the subject comprise at least two points corresponding to a nose bridge and a tip of a nose of the subject. In some embodiments, the data points associated with facial features of the subject are used to produce a 3D facial map. In some embodiments, the 3D facial map is produced using a machine learning algorithm. In some embodiments, the machine learning algorithm calculates the position of a facial feature based on an image not present in the captured one or more images. In some embodiments, the marker comprises at least two submarkers which indicate a configuration state of the device. In some embodiments, the method further comprises determining a configuration state of the device based upon the position of the marker. In some embodiments, the method further comprises determining an alignment state of the device based upon the position of the marker relative to one or more subject facial features, the one or more subject indicia, or combinations thereof. In some embodiments, the 3D facial map comprises facial landmarks not directly measured by the camera, wherein the facial landmarks not directly measured by the camera are extrapolated using the machine learning algorithm and input based on the one or more data points. In some embodiments, the method further comprises providing a side profile of the subject to the machine learning algorithm. In some embodiments, the side profile is used to calculate the nose angle of the subject. In some embodiments, the nose angle is calculated using a formula

$${tan}^{- 1} ( \frac{\vert {z}_{2} - {z}_{1} \vert}{\vert {y}_{2} - {y}_{1} \vert} ) * \frac{180}{π} ,$$

by comparing a depth at a middle of the nose z1 and a depth at top of nose z2, where (x1, y1, z1) and (x2, y2, z2) are the Cartesian-coordinates of the two points, respectively. In some embodiments, the method further comprises measuring the nose angle of the subject by comparing the tip of the nose of the subject with a depth of the nose of the subject at a top of the nose bridge of the nose. In some embodiments, measuring the nose angle of the subject is with an accuracy of from about +/−2 degrees to about +/−15 degrees. In some embodiments, the method further comprises comparing a position of the device in the nasal cavity of the subject relative to a database of video or images files of a device in a nasal cavity of the subject, and indicating a proper or improper alignment state of the device based upon similarity in one or more features between a captured image or video file of the subject, and the database of video or image files. In some embodiments, the position of the device is determined by applying a bounding box around the marker. In some embodiments, the position is determined based upon a pitch or a yaw of the device. In some embodiments, the pitch or the yaw of the device is calculated based upon the shape of the bounding box. In some embodiments, the method further comprises using a position of the housing compared to the position of the first fiducial marker to calculate a pitch of the device. The method of any of the preceding claims, wherein the pitch or the yaw of the device is calculated based upon a measurement of the marker to a projection of a plane defined by the measurement of the marker. In some embodiments, the projection of the plane defined by the known measurement of the marker is captured as a 3D object projected into a 2D space. In some embodiments, the pitch of the device is calculated based on the projection onto the plane using a formula:

$${cos}^{- 1} ( \frac{observed height}{observed width} ) * \frac{180}{π} ,$$

observed height and observed width are a height and width of the marker. In some embodiments, the camera comprises a depth camera, wherein the pitch of the device is calculated based upon the projection onto the plane using a formula

$${sin}^{- 1} ( \frac{\vert {z}_{2} - {z}_{1} \vert}{λ} ) * \frac{180}{π} ,$$

wherein λ is an actual height of the fiducial marker, wherein z1 is a depth at a top of the marker and z2 is a depth at the bottom of the marker. In some embodiments, wherein the pitch of the device is calculated with an accuracy of from about +/−2° to about +/−15° when the pitch is greater than 20°, or from about +10° to about +/−15° when the pitch is 20° or is lower than 20°. In some embodiments, the accuracy of the pitch relative to the nose of the subject is calculated based on an accuracy of the pitch of the device and an accuracy of the pitch of the nose. In some embodiments, the yaw of the device is calculated based upon a vertical edge of the bounding box. In some embodiments, the yaw of the device is calculated based upon the projection onto the plane using a formula

$${tan}^{- 1} ( \frac{{x}_{1} - {x}_{2}}{{y}_{1} - {y}_{2}} ) * \frac{180}{π} ,$$

wherein x1 is a horizontal coordinate at a top corner of the marker, x2 is a horizontal coordinate at a bottom corner of the marker, y1 is a vertical coordinate at a top corner of the marker, and y2 is a vertical coordinate at a bottom corner of the marker. In some embodiments, the yaw is calculated with an accuracy of from about +3° to about +15°. In some embodiments, the accuracy of the yaw relative to the subject's nose is calculated based on an accuracy of a yaw of the device and an accuracy of a yaw of the nose. In some embodiments, the recommendation comprises feedback to the subject as to the alignment state of the device, wherein the subject is directed to adjust alignment state of the device if it is determined the device is in an improper alignment state. In some embodiments, the method further comprises providing feedback to the subject as to the alignment state of the device, wherein the subject is directed to adjust the alignment state of the device if it is determined the device is in an improper alignment state. In some embodiments, the method further comprises providing feedback as to the alignment state of the device, wherein the subject is provided an indication of a proper alignment state of the device prior to actuating the device. In some embodiments, the method further comprises continuously tracking and continuously calculating the position of the one or more subject facial features relative to the camera. In some embodiments, the identifying the subject comprises using a machine learning algorithm to determine a 3D facial surface map of the subject with the camera. In some embodiments, the method further comprises collecting data corresponding to facial features of the subject. In some embodiments, the method further comprises calculating an angle of the nose, an angle of the head, or position of a nasal cavity using at least a portion of the data points associated with facial features of the subject. In some embodiments, the identifying the subject comprises using a machine learning algorithm to determine a 3D facial surface of the subject with the camera without using a depth sensor. In some embodiments, the identifying the subject comprises comparing detected facial features of the subject to a known or verified image of the subject to identify the subject with at least about 70% to at least about 99% accuracy. In some embodiments, the method further comprises tracking a position of the nose or face of the subject relative to the camera, wherein tracking the position of the nose or face of the subject comprises extrapolating an angle of the nose based upon the at least two points one or more points corresponding to a nose bridge and a tip of a nose of the subject. In some embodiments, identifying the subject using one or more subject indicia comprises applying a facial recognition algorithm to identify the subject. In some embodiments, the one or more subject indicia comprises one or more facial features of the subject. In some embodiments, the identifying the subject comprises matching the one or more facial features of the subject to a data file comprising data corresponding to one or more facial features of the subject. In some embodiments, the identifying the subject comprises matching the one or more facial features of the subject to a data file comprising a verified image of the subject. In some embodiments, the machine learning algorithm isolates a face within an individual frame of a captured video from the camera for analysis. In some embodiments, identifying the subject using one or more subject indicia comprises applying a facial recognition algorithm to identify the subject by matching the one or more subject indicia to a known facial scan of the subject. In some embodiments, identifying the subject comprises instructing the subject to position a face of the subject within a target view field of the camera. In some embodiments, identifying the subject comprises indicating to the subject when the face of the subject is correctly positioned within the target view field of the camera. In some embodiments, the method further comprises continuously identifying the subject during performance of the claimed method, or continuously identifying the subject at multiple time points during performance of the claimed method. In some embodiments, the method further comprises continuously identifying the subject during the actuating of the device, verifying actuation of the device, the determining the alignment state of the device, or any combination thereof. In some embodiments, the method further comprises re-identifying the subject if the subject is determined to leave the field of view of the camera during performance of the method. In some embodiments, the method further comprises identifying the device by scanning and identifying indicia located on the device with the computing device. In some embodiments, the method further comprises verifying the device corresponds to a prescription by referencing matching the device to a data file comprising data corresponding to a prescription. In some embodiments, the method further comprises verifying the device corresponds to a prescription belonging to the subject based upon a positive device identification, a positive prescription identification, a positive subject identification, or combinations thereof. In some embodiments, the method further comprises identifying an impediment to identifying the subject or verifying the device, and prompting the subject to correct the impediment. In some embodiments, the impediment comprises the subject or the device being positioned too far away from the camera, an improper lighting state, a physical obstruction in view of the camera, an improper subject head angle, a subject position out of view of the camera, or any combination thereof. In some embodiments, the improper lighting state comprises glare reflection. In some embodiments, the method further comprises continuously identifying a position of the device during performance of the claimed method, or continuously identifying a position of the device at multiple time points during performance of the claimed method. In some embodiments, the method further comprises continuously identifying a position of the device during the actuating of the device, verifying actuation of the device, the determining the alignment state of the device, or any combination thereof. In some embodiments, the method further comprises verifying a configuration state of the device based upon whether the housing is visible. In some embodiments, the method further comprises prompting the subject to actuate the device. In some embodiments the first indicator prompts the subject to actuate the device. In some embodiments, the third indicator prompts the subject to re-actuate the device. In some embodiments, the device comprises a securing mechanism preventing actuation of the device. In some embodiments, the device is in communication with the computing device. In some embodiments, the securing mechanism is released upon verifying a positive subject identification, a positive prescription identification, a proper alignment state of the device, or combinations thereof. In some embodiments, the securing mechanism is released upon a signal transmitted from the computing device to the device. In some embodiments, the recommendation comprises one or more prompts. In some embodiments, verifying delivery of the composition to the subject comprises maintaining a continuous positive subject identification throughout the actuating the device. In some embodiments, the method further comprises identifying a negative subject identification and a positive prescription identification, and not releasing the securing mechanism, preventing actuation of the device. In some embodiments, the method further comprises identifying a negative subject identification and a positive prescription identification, and generating a report. In some embodiments, the data file comprising data corresponding to one or more facial features of the subject, the data file comprising a verified image of the subject, the data file comprising data corresponding to a prescription are stored locally on the computing device. In some embodiments, the data file comprising data corresponding to one or more facial features of the subject, the data file comprising a verified image of the subject, the data file comprising data corresponding to a prescription are stored remotely from the computing device. In some embodiments, the method further comprises capturing one or more images of the subject when the device is actuated. In some embodiments, the method further comprises capturing one or more images of the subject during the identifying the subject. In some embodiments, the marker comprises a bar code, a QR code, or other camera readable code comprising drug data, prescription data, or device identification data. In some embodiments, the marker comprises an ArUco marker. In some embodiments, the method further comprises updating the machine learning algorithm based upon a captured image or video file of the subject. In some embodiments, the method further comprises detecting an object or obstruction within a reference frame. In some embodiments, the method further comprises identifying objects or obstructions within the reference frame based upon a shape, a contour, a color, or a texture of the object or obstruction. In some embodiments, the method further comprises localizing a position of the object or obstruction within a reference frame. In some embodiments, the method further comprises measuring a distance between the camera and the subject or device using a relative scale, wherein the relative scale increases the accuracy of the measurement. In some embodiments, patient identification, detection of device positioning, detection of device actuation, and/or corrective guidance are performed simultaneously. In some embodiments, the method further comprises generating a data report comprising subject identity, device serial number, drug identity, date, time, location, a video of performance of the claimed method, a photo of performance of the claimed method, an actuation event, face position at actuation, device position at actuation, an actuation confirmation, incorrect dose alert, tampering alert or any combination thereof. In some embodiments, the method further comprises storing data in a database following performance of any step of the method. In some embodiments, the data comprises image or video data. In some embodiments, the image or video data is stored as a JSON object structure. In some embodiments, the JSON object structure comprises keys mapping the positioning state of the device, the identifying the subject, the actuating the device, or any combination thereof relative to a state of performance of the method. In some embodiments, the composition comprises a medicament, vaccine, biologic, small molecule, cell line, or therapeutic.

Aspects disclosed herein provide a device for delivering a composition, comprising: a housing transitionable from a first configuration state to a second configuration state, wherein transitioning the housing from the first configuration state to the second configuration state actuates the device; a marker readable by an electronic device, the marker being exposed in the first configuration state, the marker being at least partially obscured in the second configuration state, the marker indicating the configuration state of the device; and one or more dispensing elements for delivery of the composition upon actuation of the device. In some embodiments, the device further comprises a second housing. In some embodiments, the marker is a fiducial marker. In some embodiments, the first configuration state of the plurality of configuration states indicates an alignment of the device with respect to a target region of the subject. In some embodiments, the target region is an orifice of the subject, the mouth of the subject, the nasal cavity of the subject, an olfactory region of the subject, or the columella of the subject. In some embodiments, the marker is at least partially concealed by the housing and the housing is visible in a second configuration state of the plurality of configuration states. In some embodiments, the second housing is concealed in a second configuration state of the plurality of configuration states. In some embodiments, the marker is at least partially concealed by the housing and the housing is not visible in a third configuration state of the plurality of configuration states. In some embodiments, the second housing is not concealed in a third configuration state of the plurality of configuration states. In some embodiments, the marker is not concealed and the housing is concealed in a fourth configuration state of the plurality of configuration states. In some embodiments, the second housing is visible in a fourth configuration state of the plurality of configuration states. In some embodiments, the marker is a QR code. In some embodiments, the marker is an ArUco marker. In some embodiments, the device being in the first configuration state indicates the device can be actuated. In some embodiments, the marker comprises one or more submarkers. In some embodiments, in the second configuration state, a first submarker of the one or more submarkers is concealed by the housing and a second submarker of the one or more submarkers is not concealed by the housing and the housing is not concealed. In some embodiments, in the first configuration state, the first submarker of the one or more submarkers is not concealed, and the second submarker of the one or more submarkers is not concealed, and the housing is not concealed. In some embodiments, in the third configuration state, the first submarker of the one or more submarkers is not concealed, a second submarker of the one or more submarkers is concealed, and the housing is concealed. In some embodiments, the device is configured to transition from the first configuration state to the second configuration state when the device is partially inserted into a nasal cavity of the subject. In some embodiments, the device is configured to transition from the first configuration state to the second configuration state when the device is partially inserted into the nasal cavity of the subject at a prescribed depth and angle. In some embodiments, the marker comprises at least two submarkers which indicate a configuration state of the device. In some embodiments, a position of the marker indicates a configuration state of the device. In some embodiments, the position of the marker relative to one or more subject facial features, the one or more subject indicia, or combinations thereof indicates an alignment state of the device. In some embodiments, the pitch or the yaw of the device is calculated based upon a measurement of the marker to a projection of a plane defined by the measurement of the marker. In some embodiments, the pitch of the device is calculated based on the projection onto the plane using a formula:

$${cos}^{- 1} ( \frac{observed height}{observed width} ) * \frac{180}{π} ,$$

wherein the observed height and observed width are a height and width of the marker. In some embodiments, the pitch of the device is calculated with an accuracy of from about +/−2° to about +/−15° when the pitch is greater than 20°, or from about +10° to about +15° when the pitch is 20° or is lower than 20°. In some embodiments, the yaw of the device is calculated based upon the projection of the plane using a formula

$${tan}^{- 1} ( \frac{{x}_{1} - {x}_{2}}{{y}_{1} - {y}_{2}} ) * \frac{180}{π} ,$$

wherein x1 is a horizontal coordinate at a top corner of the marker, x2 is a horizontal coordinate at a bottom corner of the marker, y1 is a vertical coordinate at a top corner of the marker, and y2 is a vertical coordinate at a bottom corner of the marker. In some embodiments, the accuracy of the yaw relative to the subject's nose is calculated based on an accuracy of a yaw of the device and an accuracy of a yaw of the nose. In some embodiments, the device comprises a securing mechanism preventing actuation of the device. In some embodiments, the marker comprises a bar code, a QR code, or other camera readable code comprising drug data, prescription data, or device identification data. In some embodiments, the marker comprises an ArUco marker. In some embodiments, the device is in communication with a computing device. In some embodiments, the device is configured to receive a first indicator, wherein the device allows actuation of the device upon receiving the first indicator. In some embodiments, the device is configured to receive the first indicator when the device is in the first configuration state. In some embodiments, the device is configured to receive a second indicator after receiving the first indicator, wherein the device is configured to prevent actuation upon receiving the second indicator. In some embodiments, the device is configured to receive the second indicator when the device is not in the first configuration state. In some embodiments, the device is configured to receive a third indicator after receiving the second indicator, wherein the device allows actuation of the device upon receiving the third indicator. In some embodiments, the device is configured to receive the third indicator when the device is in the first configuration state. In some embodiments, the composition comprises a medicament, vaccine, biologic, small molecule, cell line, or therapeutic.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Another aspect of the present disclosure provides adaptors and adaptor kits to adapt medicament delivery devices for use with any of the systems or methods described herein.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings (also “Figure” and “FIG.” herein), of which:

FIG. 1 depicts a non-limiting example of a system for recognizing and confirming a subject in need of a composition.

FIGS. 2A-2H depict non-limiting examples of configuration states of the device for delivering a composition. (A-D) illustrate an exemplary device for delivering a composition according to some embodiments. (E-F) illustrate another device for delivering a composition according to some embodiments.

FIG. 3 shows a non-limiting example of a computing device; in this case, a device with one or more processors, memory, storage, and a network interface.

FIG. 4 shows a non-limiting example of a web/mobile application provision system; in this case, a system providing browser-based and/or native mobile user interfaces.

FIG. 5 shows a non-limiting example of a cloud-based web/mobile application provision system; in this case, a system comprising an elastically load balanced, auto-scaling web server and application server resources as well synchronously replicated databases.

FIG. 6 depicts a non-limiting example process for recognizing and confirming a subject in need of a composition.

FIG. 7A depicts a non-limiting example of a device for delivering a composition in an unactuated configuration.

FIG. 7B depicts the device shown in FIG. 7A in an actuated configuration.

FIG. 8A depicts a non-limiting example of a device for delivering a composition in an unactuated configuration.

FIG. 8B depicts the device shown in FIG. 8A in an actuated configuration.

FIG. 9A depicts a non-limiting example of a device for delivering a composition in an unactuated configuration.

FIG. 9B depicts the device shown in FIG. 9A in an actuated configuration.

FIG. 10A depicts a non-limiting example of a device for delivering a composition in an unactuated configuration.

FIG. 10B depicts the device shown in FIG. 10A in an actuated configuration.

FIG. 11A depicts a non-limiting example of a syringe device for delivering a composition in an unactuated configuration.

FIG. 11B depicts the syringe device shown in FIG. 11A in an actuated configuration.

FIG. 12A depicts a side view of a non-limiting example of an inhaler device for delivering a composition in an unactuated configuration.

FIG. 12B depicts a side view of the device shown in FIG. 12A in an actuated configuration.

FIG. 12C a front view of the device shown in FIG. 12A in an unactuated configuration.

FIG. 12D depicts a front view of the device shown in FIG. 12A in an actuated configuration.

FIG. 13A depicts a non-limiting example of a dual-inserter device for delivering a composition in an unactuated configuration.

FIG. 13B depicts the dual-inserter device shown in FIG. 13A in an actuated configuration.

FIG. 14A depicts a non-limiting example of a device for delivering a composition in an unactuated configuration.

FIG. 14B depicts the device shown in FIG. 14A in an actuated configuration.

FIG. 15A depicts a non-limiting example of a multi-dose device for delivering a composition in an unactuated configuration.

FIG. 15B depicts the device shown in FIG. 15A in a partially-actuated (dose 1 delivered) configuration.

FIG. 15C depicts the device shown in FIG. 15A in a fully-actuated (dose 2 delivered) configuration.

FIG. 16A depicts a non-limiting example of a device for delivering a composition in an unactuated configuration.

FIG. 16B depicts the device shown in FIG. 16A with an inner sleeve and an outer sleeve surrounding the actuation mechanism.

FIG. 16C depicts the device shown in FIG. 16B in an unactuated configuration, with an ArUco code on the inner sleeve.

FIG. 16D depicts the device shown in FIG. 16B in an actuated configuration, with the inner sleeve hidden behind the outer sleeve.

FIG. 16E depicts the device shown in FIG. 16B post actuation with the inner sleeve remaining hidden behind the outer sleeve and a second ArUco code visible.

FIG. 17A depicts a non-limiting example of a device for delivering a composition in an unactuated configuration with an outer sleeve, an inner sleeve, and an ArUco code visible above the inner sleeve.

FIG. 17B depicts the device shown in FIG. 17A in actuated configuration with the inner sleeve hidden behind the outer sleeve and the ArUco code remaining visible above the outer sleeve.

FIG. 17C depicts the device shown in FIG. 17A post actuation with the inner sleeve remaining hidden behind the outer sleeve and two ArUco codes visible above the outer sleeve.

FIG. 18A depicts a non-limiting example of a device for delivering a composition in an unactuated configuration with an outer sleeve, an inner sleeve, and an ArUco code visible above the inner sleeve.

FIG. 18B depicts the device shown in FIG. 18A in an actuated configuration with the inner sleeve hidden behind the outer sleeve and the ArUco code remaining visible above the outer sleeve.

FIG. 18C depicts the device shown in FIG. 18A post actuation, with push buttons on either side of the cap being actuated to return the device to its original state.

FIG. 18D depicts the device shown in FIG. 18A returned to a pre-actuated configuration with an outer sleeve, an inner sleeve, and an ArUco code visible above the inner sleeve.

FIG. 19 depicts the roll, pitch, and yaw of the device according to some embodiments. Roll is defined as a rotation about the z-axis, Pitch is defined as rotation about the x-axis, Yaw is defined as rotation about the y-axis.

### DETAILED DESCRIPTION

Medicaments may be prescribed to subjects for a variety of reasons, and the prescription of controlled medication is becoming increasingly common as new drugs and indications are approved, and knowledge of the viability and efficacy of such therapies becomes well known. However, many controlled medications prescribed to subject are not always administered to the subject who holds the prescription, and drug diversion is a serious concern. In addition, verification of adherence to programs for clinical trials, treatment programs, or similar processes which require a patient to follow a prescribed dosage schedule is desirable, for example, verifying administration, verifying administration of the correct doses at the proper times, or correct dosing protocol, can improve resulting data. Accordingly, there is a need in the art to properly verify subject identity and proper medicament administration, for example, tracking guiding, confirming, and reporting proper delivery in an effort to prevent unauthorized diversion of controlled mediation, and otherwise collect data verifying proper delivery of a medication to a subject.

Systems and methods described herein include devices that allow for verifying the identity of subjects in need of certain medicaments as well as verifying that a subject in need is in the position to take a medicament before the medicament is administered. Accordingly, by identifying a subject on screen and determining that the device is in position to be administered to the subject on screen, systems and methods as described herein help prevent any misuse of medicaments and ensure that the subject in need is the one that the medicament is administered or record misuse of the medicaments in an effort to ensure the appropriate subject's receive necessary care. In particular, in some embodiments, the subject may be identified through facial recognition, while the device may be placed in a position to administer the medicament. The device may have a marker that may be identified in one or more ways in order to identify a configuration state of the device, such as a configuration state where the device is in a position to administer the medicament, a configuration state where the device is not in a position to administer the medicament, or a configuration state where the medicament has already been administered.

For example, configuration states of the device may be used to determine that the device is in a position to be actuated as well as determining if the device has been actuated. With the use of a marker and one or more housings of the device, a position of the device with respect to the face may be determined, thereby indicating if the device is in a position to be actuated (e.g., if the device is correctly positioned in the nose, and thus, ready to deliver the medicament) or not. Accordingly, in some embodiments, once the device is actuated, the marker will still be partially concealed and the housing will also be concealed, indicating that the device has been actuated. In some embodiments, once the device is actuated, the marker will be concealed and a second housing will be visible, indicating that the device has been actuated. Thus, the devices movement between each configuration state allows for determining when the device is ready to be actuated, and when actuation has occurred. In some embodiments, by monitoring an alignment region of a subject, a computing device can determine whether the device is correctly positioned for actuation. By monitoring the device, its configuration states, and the subject receiving the medicament, a computing device (e.g., a smartphone) can determine not only that the correct person is receiving the medicament, but that the device was properly actuated and that the medicament was properly received. Accordingly, accurate records of the medicament being delivered properly can be kept, providing a more accurate record for medical personnel to keep track of prescriptions and how they are applied.

In some embodiments, the device may not be a smart device, and the computing device may keep track of both the subject receiving the medicament (as opposed to another person), and when the medicament is properly received. In some embodiments, the device may be a smart device which may be able to communicate with the computing device. In some embodiments, the computing device may determine that the subject or position are incorrect, the computing device may provide an indicator to the device that causes the device to prevent actuation entirely, and prevent delivery of the medicament.

Accordingly, the systems and methods described herein improve upon conventional methods by preventing misuse of medicaments and ensuring that subjects in need receive their needed medicaments.

### Systems

Disclosed herein, in some embodiments, a subject, composition, and device verification system.

### Devices

Disclosed herein is a device. In some embodiments, the device comprises a compound delivery component, a housing, a second housing, a marker, or any combination thereof. In some embodiments, the device further comprises a compound. In some embodiments, the compound delivery component comprises the compound. In some embodiments, the compound is a medicament. In some embodiments, the compound delivery component is designed to deliver a compound to a target region of a subject. In some embodiments, the target region is an orifice of a subject. In some embodiments, the target region is a mouth of a subject. In some embodiments, the target region is the nasal cavity of a subject. In some embodiments, the target region is an olfactory region of a subject.

In some embodiments, the device comprises a marker. In some embodiments, the marker comprises a plurality of submarkers. In some embodiments, the marker comprises one or more submarkers. In some embodiments, the marker is used for recognition of the device. In some embodiments, the marker is used for verification of the device. In some embodiments, the one or more submarkers are used for recognition of the device. In some embodiments, the one or more submarkers are used for verification of the device. In some embodiments, the marker is used to assess the configuration state of the device. In some embodiments, the submarker is used to assess the configuration state of the device. In some embodiments, the one or more submarkers are used to assess the configuration state of the device.

In some embodiments, the device comprises a housing. In some embodiments, the housing is used to assess the configuration state of the device. In some embodiments, the device further comprises a second housing. In some embodiments, the second housing is used to assess the configuration state of the device. In some embodiments, the configuration state assessment comprises assessing the marker, one or more submarkers, the housing, the second housing, or any combination thereof. In some embodiments, the configuration state allows for delivery of a compound to a target region of a subject. In some embodiments, the configuration state that allows for delivery of a compound to a target region of a subject is a first configuration state (e.g., an unactuated configuration state). In some embodiments, the configuration state does not allow for delivery of a compound to a target region of a subject. In some embodiments, the device is in communication with the one or more devices listed herein. In some embodiments, the device is in communication with the one or more systems listed herein.

For example, the marker of the device may be recognized by one or more processing devices (e.g., a smartphone or other computing device). In some embodiments, recognition of the marker allows the processing device to determine what configuration state the device is in. For example, in a first configuration state (e.g. an unactuated configuration state), the marker is not concealed and the housing is showing, which the processing device may recognize as indicating the device has not been actuated, and the processing device may further track the position of the device with respect to a subject's face to ensure the device is in the proper position for actuation. In some embodiments, the device further comprises a second housing and the second housing is concealed in the first configuration state. In a second configuration state (e.g., an unactuated configuration state), the marker is at least partially concealed and the housing is showing, which the processing device may recognize as indicating that the device is ready for actuation but has not been actuated. In some embodiments, the device further comprises a second housing and the second housing is concealed in the second configuration state. In a third configuration state (e.g., the actuated position state), the marker is at least partially concealed and the housing is not showing, showing that the device has been actuated. In some embodiments, the device further comprises a second housing and the second housing is not concealed in the third configuration state. In a fourth configuration state (e.g., an actuation confirmation and/or not use ready configuration state), the marker is not concealed and the housing is concealed, which the processing device may recognize as indicating that the device is used. In some embodiments, the device further comprises a second housing and the second housing is not concealed in the fourth configuration state. In some embodiments, the device may be configured to communicate with the processing device, and may further be configured to prevent actuation of the device. The device preventing actuation may occur when the device is in the second configuration state but not in the proper position or when the device is in the third configuration state. In some embodiments, the device may not be configured to communicate with the processing device. In some embodiments, the processing device may track actuation of the device based on a schedule of proper actuations for the subject (e.g., a prescribed schedule for delivery of the medicament) and/or identification of one or more configuration states of the device (e.g., the device being in the proper position in the first configuration state, the device being actuated once in the second configuration state, and then the device being present in the third configuration state.)

In some embodiments, the housing comprises a first housing marker. In some embodiments, the first housing marker comprises an annular ring. In some embodiments, the first housing marker comprises a color distinct from the housing. In some embodiments, the second housing comprises a second housing marker. In some embodiments, the second housing marker comprises a color distinct from the first housing marker.

In some embodiments, the marker on the device is a fiducial marker. In some embodiments, the marker on the device is a non-fiducial marker. In some embodiments, the marker is a QR code. In some embodiments, the marker is an ArUco marker. In some embodiments, the marker has one or more submarkers. In some embodiments, a configuration state of the device and the subject is based upon the position of the marker. In some embodiments, the alignment state of the device (also referred to herein as a “position of the device”) and the subject is based upon a position of the marker relative to one or more subject facial features. In some embodiments, the alignment state of the device and the subject is based upon a position of the marker relative to one or more criteria. In some embodiments, the alignment state of the device and the subject is based upon a position of the marker relative to one or more subject facial features and the one or more criteria.

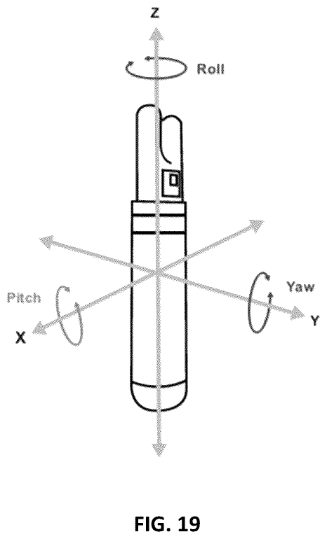

Aspects disclosed herein provide a device for delivering a composition, comprising: a housing transitionable from a first configuration state to a second configuration state, wherein transitioning the housing from the first configuration state to the second configuration state actuates the device; a marker readable by an electronic device, the fiducial marker being exposed in the first configuration state, the fiducial marker being at least partially obscured in the second configuration state, the fiducial marker indicating the configuration state of the device; and one or more dispensing elements for delivery of the composition upon actuation of the device. In some embodiments, the marker is a fiducial marker. In some embodiments, the first configuration state of the plurality of configuration states indicates an alignment of the device with respect to a target region of the subject. In some embodiments, the first configuration state of the plurality of configuration states indicates an alignment of the device with respect to an alignment region of the subject. In some embodiments, the target region and/or the alignment region is an orifice of the subject, the mouth of the subject, the nasal cavity of the subject, an olfactory region of the subject, or the columella of the subject. In some embodiments, the device is configured to be partially inserted into the nasal cavity of the subject. In some embodiments, partial insertion of the device into the nasal cavity of the subject comprises pressing the device against a columella of the subject. In some embodiments, the marker is at least partially concealed by the housing and the housing is visible in a second configuration state of the plurality of configuration states. In some embodiments, the marker is at least partially concealed and the housing is not visible in a third configuration state of the plurality of configuration states. In some embodiments, the second housing is visible in a third configuration state of the plurality of configuration states. In some embodiments, the marker is not concealed and the housing is concealed in a fourth configuration state of the plurality of configuration states. In some embodiments, the second housing is visible in a fourth configuration state of the plurality of configuration states. In some embodiments, the marker is a QR code. In some embodiments, the marker is an ArUco marker. In some embodiments, the device being in the first configuration state indicates the device can be actuated. In some embodiments, the marker comprises one or more submarkers. In some embodiments, in the second configuration state, a first submarker of the one or more submarkers is concealed by the housing and a second submarker of the one or more submarkers is not concealed by the housing and the housing is not concealed. In some embodiments, in the first configuration state, the first submarker of the one or more submarkers is not concealed, and the second submarker of the one or more submarkers is not concealed, and the housing is not concealed. In some embodiments, in the third configuration state, the first submarker of the one or more submarkers is not concealed, a second submarker of the one or more submarkers is concealed, and the housing is concealed. In some embodiments, in the third configuration state, the marker is concealed, the housing is concealed, and the second housing is not concealed. In some embodiments, in the fourth configuration state, the marker is not concealed and the housing is concealed. In some embodiments, in the fourth configuration state, the second housing is not concealed. In some embodiments, the device being in the fourth configuration state indicates actuation confirmation. In some embodiments, the device being in the fourth configuration state indicates the device is not use ready, for example, it has been used and/or it is not loaded with the composition. In some embodiments, the device is configured to transition from the first configuration state to the second configuration state when the device is partially inserted into a nasal cavity of the subject. For example, in some embodiments, as the device is inserted into the nasal cavity of the subject, the device is pressed against the columella of the subject, thereby preventing further insertion of the device. Accordingly, the device is partially inserted when the device transition from the first configuration state to the second configuration state in some embodiments. In some embodiments, the columella of the subject prevents full insertion of the device. In some embodiments, the device is configured to transition from the first configuration state to the second configuration state when pressure is applied to the device by the subject's columella. In some embodiments, the device is configured to transition from the first configuration state to the second configuration state when the device is partially inserted into the nasal cavity of the subject at a prescribed depth and angle. In some embodiments, the marker comprises at least two submarkers which indicate a configuration state of the device. In some embodiments, a position of the marker indicates a configuration state of the device. In some embodiments, the position of the marker relative to one or more subject facial features, the one or more subject indicia, or combinations thereof indicates an alignment state of the device. The pitch, yaw, and roll of the device is illustrated in FIG. 19. In some embodiments, the pitch or the yaw of the device is calculated based upon a measurement of the marker to a projection of a plane defined by the measurement of the marker. In some embodiments, the pitch of the device is calculated based on the projection onto the plane using a formula:

$${cos}^{- 1} ( \frac{observed height}{observed width} ) * \frac{180}{π} ,$$

wherein the observed height and observed width are a height and width of the marker. In some embodiments, the pitch of the device is calculated with an accuracy of +/−2°, +/−3°, +/−4°, +/−5°, +/−6°, +/−7°, +/−8°, +/−9°, +/−10°, +/−11°, +/−12°, +/−13°, +/−14°, or +/−15° when the pitch is greater than 20°, or ±10°, +11°, +12°, +13°, +14° or ±15° when the pitch is or is lower than 20°. In some embodiments, the pitch of the device is calculated with an accuracy of +/−2° when the pitch is greater than 20°. In some embodiments, the pitch of the device is calculated with an accuracy of +/−3° when the pitch is greater than 20°. In some embodiments, the pitch of the device is calculated with an accuracy of +/−4° when the pitch is greater than 20°. In some embodiments, the pitch of the device is calculated with an accuracy of +/−5° when the pitch is greater than 20°. In some embodiments, the pitch of the device is calculated with an accuracy of +/−6° when the pitch is greater than 20°. In some embodiments, the pitch of the device is calculated with an accuracy of +/−7° when the pitch is greater than 20°. In some embodiments, the pitch of the device is calculated with an accuracy of +/−8° when the pitch is greater than 20°. In some embodiments, the pitch of the device is calculated with an accuracy of +/−9° when the pitch is greater than 20°. In some embodiments, the pitch of the device is calculated with an accuracy of +/−10° when the pitch is greater than 20°. In some embodiments, the pitch of the device is calculated with an accuracy of +/−11° when the pitch is greater than 20°. In some embodiments, the pitch of the device is calculated with an accuracy of +/−12° when the pitch is greater than 20°. In some embodiments, the pitch of the device is calculated with an accuracy of +/−13° when the pitch is greater than 20°. In some embodiments, the pitch of the device is calculated with an accuracy of +/−14° when the pitch is greater than 20°. In some embodiments, the pitch of the device is calculated with an accuracy of +/−15° when the pitch is greater than 20°. In some embodiments, the pitch of the device is calculated with an accuracy of +/−10° when the pitch is 20° or is lower than 20°. In some embodiments, the pitch of the device is calculated with an accuracy of +/−11° when the pitch is 20° or is lower than 20°. In some embodiments, the pitch of the device is calculated with an accuracy of +/−12° when the pitch is 20° or is lower than 20°. In some embodiments, the pitch of the device is calculated with an accuracy of +/−13° when the pitch is 20° or is lower than 20°. In some embodiments, the pitch of the device is calculated with an accuracy of +/−14° when the pitch is 20° or is lower than 20°. In some embodiments, the pitch of the device is calculated with an accuracy of +/−15° when the pitch is 20° or is lower than 20°. In some embodiments, the yaw of the device is calculated based upon the projection onto the plane using a formula

$${tan}^{- 1} ( \frac{{x}_{1} - {x}_{2}}{{y}_{1} - {y}_{2}} ) * \frac{180}{π} ,$$

wherein x1 is a horizontal coordinate at a top corner of the marker, x2 is a horizontal coordinate at a bottom corner of the marker, y1 is a vertical coordinate at a top corner of the marker, and y2 is a vertical coordinate at a bottom corner of the marker. In some embodiments, the accuracy of the yaw relative to the subject's nose is calculated based on an accuracy of a yaw of the device and an accuracy of a yaw of the nose. In some embodiments, the device comprises a securing mechanism preventing actuation of the device. In some embodiments, the marker comprises a bar code, a QR code, or other camera readable code comprising drug data, prescription data, or device identification data. In some embodiments, the marker comprises an ArUco marker. In some embodiments, the device is in communication with a computing device. In some embodiments, the device is configured to receive a first indicator, wherein the device allows actuation of the device upon receiving the first indicator. In some embodiments, the device is configured to receive the first indicator when the device is in the first configuration state. In some embodiments, the device is configured to receive a second indicator after receiving the first indicator, wherein the device is configured to prevent actuation upon receiving the second indicator. In some embodiments, the device is configured to receive the second indicator when the device is not in the first configuration state. In some embodiments, the device is configured to receive a third indicator after receiving the second indicator, wherein the device allows actuation of the device upon receiving the third indicator. In some embodiments, the device is configured to receive the third indicator when the device is in the first configuration state. In some embodiments, the composition comprises a medicament, vaccine, biologic, small molecule, cell line, or therapeutic. In some embodiments, the device further comprises an anti-tampering mechanism. In some embodiments, the device further comprises a tamper sensor. In some embodiments, the device further comprises a tamper-evident system. In some embodiments, the device further comprises a tamper indication system.

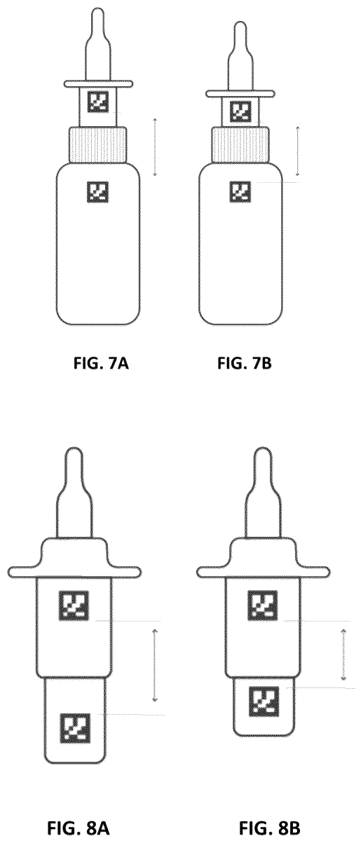

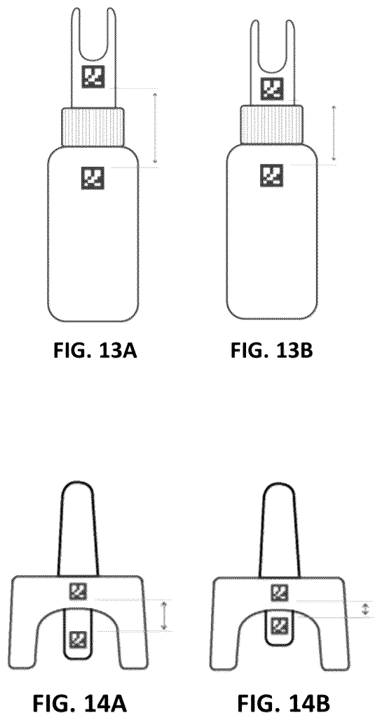

FIGS. 7-18 depict non-limiting examples of a device according to the present disclosure.

### Computer Systems

Disclosed herein are computer systems configured to verify a subject, verify a composition, and verify the subject is due for receiving the composition. In some embodiments, computer systems are configured to verify a subject, verify a composition, and verify the subject is in need of the composition. In some embodiments, the computer system comprises one or more processors configured to execute instructions for performing the methods disclosed herein. In some embodiments, the one or more processors is configured to verify data of the subject. In some embodiments, the one or more processors is configured to verify data of the composition. In some embodiments, the one or more processors is configured to verify data of the device. The computer systems disclosed herein have one or more software modules for receiving subject, device, and composition data and verifying subject, device, and composition data.

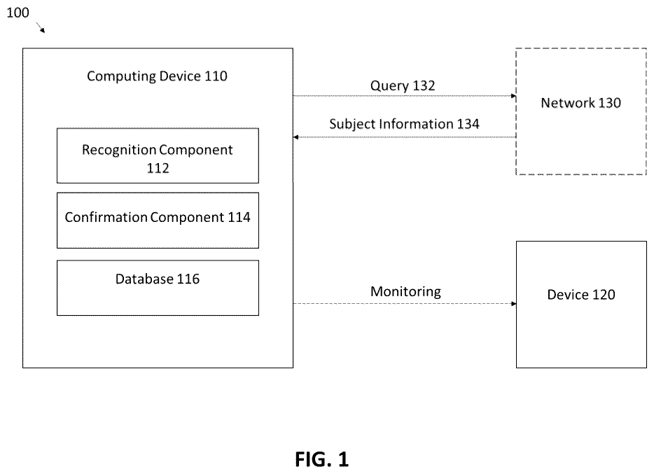

FIG. 1 depicts a non-limiting example of a computer system 100 for verifying a subject, verifying a composition, and verifying the subject is due for receiving the composition. In this depicted example, system 100 includes computing device 110, device 120, and network 130.

In this depicted embodiment, computing device 110 further includes recognition component 112, confirmation component 114, and database 116. In this depicted embodiment, computing device 110 is configured to communicate with device 120 and network 130. In this depicted embodiment, computing device 110 may query 132 the network 130. In some embodiments, the computing device 110 queries 132 the network 130 for subject information 134. In this depicted embodiment, the network 130 may send subject information 134 to computing device 110. In this depicted embodiment, the computing device 110 is further configured to monitor device 120.

In this depicted embodiment, computing device 110 further comprises recognition component 112. In some embodiments, the recognition component 112 includes or may access a camera. In some embodiments, the recognition component 112 is in communication with a camera. In some embodiments, recognition component 112 may receive one or more images from the camera. In some embodiments, the camera is a part of computing device 110. In some embodiments, the camera is connected to computing device 110. In some embodiments, the camera is connected to recognition component 112. In some embodiments, the recognition component 112 is configured to monitor the face of a subject. In some embodiments, the recognition component 112 is configured to monitor device 120. In some embodiments, the recognition component 112 is configured to monitor a marker of device 120. In some embodiments, the recognition component 112 is configured to monitor a housing of device 120. In some embodiments, the recognition component 112 is configured to monitor a marker and a housing of device 120. In some embodiments, the recognition component 112 is configured to monitor the device 120 and the subject. In some embodiments, recognition component 112 recognizes the face of a subject. In some embodiments, recognition component 112 recognizes device 120. In some embodiments, recognition component 112 recognizes a marker of device 120. In some embodiments, recognition component 112 recognizes a housing of device 120. In some embodiments, the recognition component 112 is configured to recognize a marker and a housing of device 120. In some embodiments, recognition component 112 recognizes the face of a subject and device 120. In some embodiments, recognition component 112 does not recognize the face of a subject for at least a period of time while the computing device 110 is monitoring the subject and/or the device 120. In some embodiments, recognition component 112 does not recognize device 120 for at least a period of time while the computing device 110 is monitoring the subject and/or the device 120. In some embodiments, recognition component 112 does not recognize a marker of device 120 for at least a period of time while the computing device 110 is monitoring the subject and/or the device 120. In some embodiments, recognition component 112 does not recognize a housing of device 120 for at least a period of time while the computing device 110 is monitoring the subject and/or the device 120. In some embodiments, recognition component 112 does not recognize the face of a subject or device 120 for at least a period of time while the computing device 110 is monitoring the subject and/or the device 120. In some embodiments, recognition component 112 does not recognize the face of a subject because a person who is not the subject is attempting to use the device (e.g., the face of the person who is attempting the recognition component is not the subject who is being monitored). In some embodiments, recognition component 112 does not recognize the face of a subject if the subject cannot be viewed by the recognition component while the subject is being monitored (e.g., if the subject turns their face, moves out of camera, or obscures or conceals their face). In some embodiments, recognition component 112 does not recognize the face of a subject if the subject is not the subject intended to use the system and the device does not administer a compound. In some embodiments, recognition component 112 does not recognize device 120 if the device is not the device intended to be used with the subject because the recognition component cannot identify one or more submarkers of the marker. In some embodiments, recognition component 112 does not recognize a marker of device 120 if the device is not the device intended to be used with the subject. As a non-limiting example, a subject using computing device 110 has one or more images captured by recognition component 112 and does not match the subject information 134 comprising one or more images stored in network 130 or database 116. In some embodiments, when the subject does not match the subject information 134 comprising one or more images stored in network 130 or database 116, the device will not administer the compound. In some embodiments, when the subject does not match the subject information 134 comprising one or more images stored in network 130 or database 116, the computing device 110 may display a recommendation indicating that the compound should not be administered (e.g., because the subject does not match subject information 134). In some embodiments, when the subject does not match the subject information 134 comprising one or more images stored in network 130 or database 116, the computing device 110 may record that an unauthorized user attempted to use the compound. As another non-limiting example, a subject using computing device 110 has one or more images captured by recognition component 112 and does match the subject information 134 comprising one or more images stored in network 130 or database 116; however, the device 120 is not recognized as the device associated with the subject. In a non-limiting example, a subject using the system has a device with submarkers specific to them and they accidently take the device of another user of the system. When the subject goes to use the system to deliver the compound, the system does not recognize the other device accidently acquired by the subject. In some embodiments, when the device is not recognized for use with this subject, the device will not administer the compound. In some embodiments, when the device is not recognized for use with this subject, the computing device 110 may display a recommendation indicating that the compound should not be administered (e.g., because the incorrect device would be used). In yet another non-limiting example, the subject (e.g., subject identity) is confirmed and the device is confirmed as the device for use by the subject; however, the subject is not in need of the compound (e.g., because the subject is not due to receive another dose of the compound based on their prescription schedule). In some embodiments, when the subject is confirmed to not be in need of the compound, the device will not administer the compound. In some embodiments, when the subject is confirmed to not be in need of the compound, the computing device 110 may display a recommendation indicating that the compound should not be administered (e.g., because subject is not in need of the compound). A non-limiting example of a subject not in need of the compound includes a subject trying to have the compound administered at an earlier time point than is prescribed by the system.

In this depicted embodiment, computing device 110 further comprises confirmation component 114. In some embodiments, confirmation component 114 confirms the subject being monitored by recognition component 112 is the correct subject. In some embodiments, confirmation component 114 confirms device 120 being monitored by recognition component 112 is the correct device 120. In some embodiments, the computing device 110 queries 132 the network 130 to receive subject information 134 for the confirmation component 114 to assess the subject monitored by recognition component 112. In some embodiments, the subject information 134 is used by confirmation component 114 to verify the subject monitored by recognition component 112 is the correct subject. In some embodiments, the subject information 134 is used by confirmation component 114 to verify the subject monitored by recognition component 112 is the incorrect subject. In embodiments where the device 120 is configured to communicate with the computing device 110, the computing device 110 may provide an indication to administer the compound upon verification of the correct subject by confirmation component 114, verification of the device associated with the subject, and verification of the subject being in need of the compound. In embodiments where the device 120 is configured to communicate with the computing device 110, the computing device may provide an indication to not administer the compound upon verification of the incorrect subject by confirmation component 114, which may result in device 120 not allowing administration of the compound. In embodiments where the device 120 is configured to communicate with the computing device 110, the computing device may provide an indication to not administer the compound upon verification of the incorrect device by confirmation component 114, which may result in device 120 not allowing administration of the compound. In embodiments where the device 120 is configured to communicate with the computing device 110, the computing device may provide an indication to not administer the compound upon verification of the subject not being in need of the compound, which may result in device 120 not allowing administration of the compound. In embodiments where the device 120 is configured to communicate with the computing device 110, the computing device 110 may display a recommendation to administer or not administer the compound consistent with the indication provided to the device 110. In embodiments where the device 120 is not configured to communicate with the computing device 110, the computing device 110 may display a recommendation to administer the compound upon verification of the correct subject by confirmation component 114, verification of the device associated with the subject, and verification of the subject being in need of the compound. In embodiments where the device 120 is not configured to communicate with the computing device 110, the computing device 110 may display a recommendation to not administer the compound upon verification of the incorrect subject by confirmation component 114. In embodiments where the device 120 is not configured to communicate with the computing device 110, the computing device 110 may display a recommendation to not administer the compound upon verification of the incorrect device by confirmation component 114. In embodiments where the device 120 is not configured to communicate with the computing device 110, the computing device 110 may display a recommendation to not administer the compound upon verification of the subject not being in need of the compound.

In this depicted embodiment, computing device 110 further comprises database 116. In some embodiments, database 116 comprises subject information, e.g., subject information 134. In some embodiments, computing device 110 queries 132 network 130 to receive subject information 134. In some embodiments, subject information 134 is saved to database 116 of computing device 110. In some embodiments, database 116 is accessed by confirmation component 114. In some embodiments, confirmation component 114 confirms the subject being monitored by recognition component 112 is the correct subject with subject information 134 stored on database 116. In some embodiments, the subject information 134 stored on database 116 is used by confirmation component 114 to assess the subject monitored by recognition component 112. In some embodiments, the subject information, stored on database 116 is used by confirmation component 114 to verify the subject monitored by recognition component 112 is the correct subject. In some embodiments, the subject information 134 stored on database 116 is used by confirmation component 114 to verify the subject monitored by recognition component 112 is the incorrect subject.

In some embodiments, verification of the correct subject by confirmation component 114 through the subject information 134 allows device 120 to administer the compound if the device and the subject being in need of the compound are also verified. In some embodiments, the device 120 may be a “smart device” configured to communicate with computing device 110. In some embodiments where the device 120 is configured to communicate with computing device 110, verification of the correct subject by confirmation component 114 through the subject information 134 allows manual activation of device 120 to administer the compound if the device and the subject being in need of the compound are also verified. In some embodiments where the device 120 is configured to communicate with computing device 110, verification of the correct subject by confirmation component 114 through the subject information 134 allows device 120 to administer the compound without manual activation if the device and the subject being in need of the compound are also verified. In some embodiments where the device 120 is configured to communicate with computing device 110, verification of the correct subject and that the device is in the correct configuration state by confirmation component 114 through the subject information 134 results in computing device 110 providing an indicator to device 120 to deliver a compound if the device and the subject being in need of the compound are also verified. In some embodiments where the device 120 is configured to communicate with computing device 110, verification of the incorrect subject by confirmation component 114 through the subject information 134 results in device 120 not being able to administer the compound. In some embodiments where the device 120 is configured to communicate with computing device 110, verification of the incorrect subject by confirmation component 114 through the subject information 134 results in computing device 110 providing an indicator to device 120 to deliver a compound, if the device and the subject being in need of the compound are also verified. In some embodiments, the subject information 134 comprises one or more subject identifying parameters. In some embodiments, the one or more subject identifying parameters may comprise biometric features of the subject (e.g., unique factial features) which are measured by the computing device 110 and stored as a hash. In some embodiments, the hash may be used to verify subject identity. In some embodiments, the subject information 134 comprises one or more images of the subject. In some embodiments, the subject information 134 comprises a prescription of a subject. In some embodiments, the subject information 134 comprises a birthdate of a subject. In some embodiments, the subject information 134 comprises a name of a subject. In some embodiments, the subject information 134 comprises a facial map of the subject. In some embodiments, subject information 134 is sufficient for confirmation component 114 to verify the subject monitored by recognition component 112. In some embodiments where the device 120 is configured to communicate with computing device 110, computing device 110 may provide an indicator indicating that device 120 should administer the composition if computing device 110 determines that the subject information 134 is sufficient and if the device and the subject being in need of the compound are also verified. In some embodiments where the device 120 is configured to communicate with computing device 110, computing device 110 may provide an indicator indicating that the device should not administer the composition if the computing device determines that the subject information is insufficient. In some embodiments where the device 120 is configured to communicate with computing device 110, computing device 110 may determine the subject information 134 is sufficient if one or more parameters of the subject information 134 is confirmed by the confirmation component 114 or recognition component 112. In some embodiments where the device 120 is configured to communicate with computing device 110, computing device 110 may determine the subject information 134 is insufficient if one or more parameters of subject information 134 is not confirmed by confirmation component 114 or recognition component 112. In some embodiments, computing device 110 does not query 132 network 130 for subject information 134 when the subject information 134 stored on database 116 is sufficient for confirmation component 114 to verify the subject monitored by recognition component 112. In some embodiments, the subject information 134, stored on database 116 is insufficient for confirmation component 114 to verify the subject monitored by recognition component 112. In some embodiments, computing device 110 queries 132 network 130 for subject information 134 when the subject information 134, stored on database 116 is insufficient for confirmation component 114 to verify the subject monitored by recognition component 112. In some embodiments, confirmation component 114 may apply one or more machine learning models to verify the identity of the subject. In some embodiments, the one or more machine learning models may include a classifier. In some embodiments, the classifier may receive the one or more images captured by the computing device 110 as input. In some embodiments, the classifier may determine one or more features of the subject identified in the one or more images captured by the computing device 110 and compare the one or more features identified in the one or more images captured by the computing device 110 to one or more features of the subject from subject information 134. Based on the comparison, the classifier may output a verification of the identity of the correct subject based on the one or more features of the subject identified in the one or more images captured by the computing device 110 and the one or more features of the subject from subject information 134.

In some embodiments, computing device 110 displays one or more recommendations upon verification of one or more factors by computing device 110. In some embodiments, the device 120 is not configured to communicate (e.g. provide messages to) with the computing device 110. In some embodiments where the device 120 is not configured to communicate with the computing device 110, computing device 110 may display a recommendation that the compound should be administered upon verification of the correct subject by confirmation component 114 through the subject information 134. In some embodiments where the device 120 is not configured to communicate with the computing device 110, computing device 110 may display a recommendation that the compound should be administered upon verification of the correct subject by confirmation component 114 through the subject information 134 and verification of the subject being in need of the compound. In some embodiments where the device 120 is not configured to communicate with the computing device 110, computing device 110 may display a recommendation that the compound should be administered upon verification of the correct subject based on subject information 134 and that the device is in the correct configuration state by confirmation component 114 if the subject being in need of the compound is also verified. In some embodiments where the device 120 is not configured to communicate with the computing device 110, computing device 110 may display a recommendation that the compound should not be administered upon verification of the incorrect subject by confirmation component 114 based on the subject information 134. In some embodiments where the device 120 is not configured to communicate with the computing device 110, computing device 110 may display a recommendation that the compound should not be administered upon verification of the incorrect subject by confirmation component 114 based on the subject information 134, the incorrect device by confirmation component 114, and/or verification of the subject not being in need of the compound. In some embodiments, the subject information 134 comprises one or more parameters. In some embodiments, the subject information 134 comprises one or more images of the subject. In some embodiments, the subject information 134 comprises a prescription of a subject. In some embodiments, the subject information 134 comprises a birthdate of a subject. In some embodiments, the subject information 134 comprises a name of a subject. In some embodiments, the subject information 134 comprises a facial map of the subject. In some embodiments, subject information 134 is sufficient for confirmation component 114 to verify the subject monitored by recognition component 112. In some embodiments where the device 120 is not configured to communicate with the computing device 110, computing device 110 may display a recommendation indicating that the device should not administer the composition if the computing device determines that the subject information is insufficient. In some embodiments where the device 120 is not configured to communicate with the computing device 110, computing device 110 may determine the subject information 134 is sufficient if one or more parameters of the subject information 134 is confirmed by the confirmation component 114 or recognition component 112. In some embodiments where the device 120 is not configured to communicate with the computing device 110, computing device 110 may determine the subject information 134 is insufficient if one or more parameters of subject information 134 is not confirmed by confirmation component 114 or recognition component 112. In some embodiments, computing device 110 does not query 132 network 130 for subject information 134 when the subject information 134 stored on database 116 is sufficient for confirmation component 114 to verify the subject monitored by recognition component 112. In some embodiments, the subject information 134, stored on database 116 is insufficient for confirmation component 114 to verify the subject monitored by recognition component 112. In some embodiments, computing device 110 queries 132 network 130 for subject information 134 when the subject information 134, stored on database 116 is insufficient for confirmation component 114 to verify the subject monitored by recognition component 112.

In some embodiments, the marker on the device is a fiducial marker. In some embodiments, the marker on the device is a non-fiducial marker. In some embodiments, the marker is a QR code. In some embodiments, the marker is an ArUco marker. In some embodiments, the marker has one or more submarkers. In some embodiments, an configuration state of the device and the subject is based upon the position of the marker. In some embodiments, the alignment state of the device and the subject is based upon a position of the marker relative to one or more subject facial features. In some embodiments, the alignment state of the device and the subject is based upon a position of the marker relative to one or more criteria. In some embodiments, the alignment state of the device and the subject is based upon a position of the marker relative to one or more subject facial features and the one or more criteria.

In some embodiments, device 120 is in communication with computing device 110. In this depicted embodiment, device 120 may be activated by computing device 110. In this depicted embodiment, device 120 may be deactivated by computing device 110. In some embodiments, device 120 being monitored by recognition component 112 of computing device 110 may be unable to administer a compound upon the absence of verification by confirmation component 114. In some embodiments, device 120 is monitored by computing device 110, e.g., FIG. 1. In this depicted embodiment, device 120 being monitored by recognition component 112 of computing device 110 may be manually activated upon verification by confirmation component 114, if the subject and the subject being in need of the compound are also verified. In this depicted embodiment, device 120 being monitored by recognition component 112 of computing device 110 may be manually turned off upon the absence of verification by confirmation component 114.

In some embodiments, device 120 is not in communication with computing device 110. In those embodiments, computing device 110 may display one or more recommendations as described above. In particular, in those embodiments, when the computing device 110 verifies that one or more conditions for delivery are not met, the computing device 110 displays a recommendation to not deliver the medicament. Even further, computing device 110 may display one or more prompts in the recommendation related to the one or more conditions. For example, if the device 120 is in the first configuration state and is not in the correct position, the recommendation may include a prompt indicating that the position of device 120 needs to be adjusted. As another example, if the device 120 is in the first configuration state but the subject is not able to be monitored, the recommendation may include a prompt that the subject needs to move into view. As yet another example, if the device 120 is in the first configuration state but the incorrect subject is being monitored, the recommendation may include a prompt indicating that the correct subject needs to move into view. As indeed another example, if the device 120 is in the third configuration state due to being actuated but the subject is still being monitored by the computing device, the recommendation may include a prompt indicating that the medicament should not be delivered until a later time. As yet another example, if the device 120 is in the third configuration state due to being actuated, but the medicament was delivered to the improper subject, was delivered at an improper timing (e.g., based on the prescription schedule), or was delivered from an improper position, the recommendation may include a prompt indicating that the medicament was delivered to the improper subject, at the improper time, or at the improper position, respectively. As another example, if the device 120 is in the first configuration state, but the device is not inserted in an target region (e.g., the nasal cavity, an olfactory region, or the columella of the subject) or not inserted into a correct target region, the recommendation may include a prompt indicating that the device must be inserted into a correct target region. As even another example, if the device 120 is in the first configuration state but cannot be recognized by the computing device 110 because the device 120 is too far away, the recommendation may include a prompt indicating that the device 120 needs to be moved closer to the computing device 110. In another example, if the subject cannot be recognized by the computing device 110 because the subject is too far away or not on screen, the recommendation may include a prompt indicating that the subject needs to move closer to the computing device 110 or into the view of computing device 110. In yet another example, if the subject, device 120, or both cannot be recognized by the computing device 110 because there is not enough light to recognize the subject, device 120, or both, the recommendation may include a prompt indicating that the lighting around the subject, device 120, or both needs to be adjusted. In yet another example, if the device 120 cannot be recognized by the computing device 110 because an object (e.g., a finger) is fully or partially covering the marker, the recommendation may include a prompt indicating that an object is covering the marker. Accordingly, in embodiments, where the device 120 is not in communication with computing device 110, the device 120 may not have mechanism to prevent actuation, but can recommend proper delivery and record improper deliveries as well.

In some embodiments, the computing device 110 may be associated with a user. In some embodiments, a user may be a subject. In some embodiments, the user may be a subject in need of a composition. In some embodiments, a user may be assisted by a guardian of a user or a subject. In some embodiments, a user may be assisted by a medical professional of a user or a subject. In some embodiments, computing device 110 may be a personal electronic device of a user. In some embodiments, computing device 110 may be a personal electronic device of a subject as described herein. In some embodiments, computing device 110 may be a personal electronic device of a person associated with a user or a subject as described herein. In some embodiments, computing device 110 comprises a phone, such as a smartphone. In some embodiments, computing device 110 comprises a computer. In some embodiments, computing device 110 comprises a laptop. In some embodiments, computing device 110 comprises a tablet. In some embodiments, computing device 110 is another processing device as described herein.

In some embodiments, the device 120 is monitored by computing device 110. In some embodiments, the subject is monitored by computing device 110. In some embodiments, the device 120 and subject are monitored by computing device 110. In some embodiments, a first indicator is given. In some embodiments, a first indicator is given when the subject, device, device configuration state, and the subject being in need of the compound are verified. In some embodiments, a second indicator is given. In some embodiments, the second indicator is based on monitoring the subject, the device, or both. In some embodiments, a second indicator is given when the subject, device, device configuration state, or the subject being in need of the compound are not verified. In some embodiments, a second indicator is given when the subject, device, device configuration state, or the subject being in need of the compound are not continuously verified through active monitoring. In some embodiments the second indicator prevents the device from administering the composition. In some embodiments, the second indicator is based on the subject being unidentifiable during the monitoring. In some embodiments, the second indicator is based on the device not being in a first configuration state during monitoring. In some embodiments, the second indicator is based on the subject being unidentifiable and the device not being in the first configuration state during monitoring. For example, in some embodiments, when the subject is identified and the device is in the first configuration state, the computing device 110 may provide the first indicator indicating that the medicament is ready to be administered. However, in those embodiments, if the subject can no longer be identified and/or the device is no longer in the first configuration state, the computing device may provide the second indicator indicating that the medicament is not ready to be administered, and the device may prevent administration. In some embodiments, a third indicator is given based on monitoring. In some embodiments, a third indicator is given after the second indicator was provided. In some embodiments, the third indicator allows the device to administer the composition. In some embodiments, the third indicator is based on the subject being identifiable during the monitoring. In some embodiments, the third indicator is based on the device being in a first configuration state after previously being in a different configuration state during monitoring. In some embodiments, the third indicator is based on the subject being identifiable and the device being in the first configuration state after previously being in a different configuration state during monitoring. For example, in the examples where a second indicator is provided, the subject may once again be identified and/or the device 120 may return to the first configuration state. Upon identifying the subject again and/or confirming that the device 120 is in the first configuration state, computing device 110 may provide the third indicator indicating that the device is ready to administer the compound.

In some embodiments, the subject information comprises facial features of the subject. In some embodiments, the subject is identified by analyzing an image of one or more images that was captured. In some embodiments, the image of the one or more images is of the subject's face. In some embodiments, the subject is identified by analyzing an image of one or more images that was captured of the subject's face by associating at least 68 data points to facial features of the subject based on one or more reference images of the subject. In some embodiments, two data points of the data points associated facial features of the subject correspond to a nose bridge and a tip of a nose of the subject. In some embodiments, more than two data points of the data points associated facial features of the subject correspond to a nose bridge and a tip of a nose of the subject. In some embodiments, the data points associated with facial features of the subject are used to produce a 3D facial map. In some embodiments, the 3D facial map is produced using a machine learning algorithm. In some embodiments, the machine learning algorithm calculates the position of a facial feature. In some embodiments, the machine learning algorithm does not directly measure the position of a facial feature. In some embodiments, the 3D facial map comprises facial landmarks and/or facial features imaged. In some embodiments, the measurements of the facial landmarks and/or facial features are extrapolated using the machine learning algorithm. In some embodiments, the extrapolated measurements are input as one or more data points.

In some embodiments, identifying the subject comprises using a machine learning algorithm to determine a 3D facial surface map of the subject with the camera based on one or more images of a subject. In some embodiments, the one or more images may be retrieved from a network. In some embodiments, data is collected corresponding to facial features of the subject based on the one or more images of the subject. In some embodiments, an angle of the nose, an angle of the head, or position of a nasal cavity is calculated using at least a portion of the data points associated with facial features of the subject. In some embodiments, identifying the subject comprises using a machine learning algorithm to determine a 3D facial surface of the subject with the camera without using a depth sensor. In some embodiments, identifying the subject comprises comparing detected facial features of the subject to a known or verified image of the subject to identify the subject with at least about 70%, at least about 75%, at least about 80%, at least about 85%, at least about 90%, at least about 95%, at least about 98%, or at least about 99% accuracy. In some embodiments, identifying the subject comprises comparing detected facial features of the subject to a known or verified image of the subject to identify the subject with at least about 70% accuracy. In some embodiments, identifying the subject comprises comparing detected facial features of the subject to a known or verified image of the subject to identify the subject with at least about 75% accuracy. In some embodiments, identifying the subject comprises comparing detected facial features of the subject to a known or verified image of the subject to identify the subject with at least about 80% accuracy. In some embodiments, identifying the subject comprises comparing detected facial features of the subject to a known or verified image of the subject to identify the subject with at least about 85% accuracy. In some embodiments, identifying the subject comprises comparing detected facial features of the subject to a known or verified image of the subject to identify the subject with at least about 90% accuracy. In some embodiments, identifying the subject comprises comparing detected facial features of the subject to a known or verified image of the subject to identify the subject with at least about 95% accuracy. In some embodiments, identifying the subject comprises comparing detected facial features of the subject to a known or verified image of the subject to identify the subject with at least about 98% accuracy. In some embodiments, identifying the subject comprises comparing detected facial features of the subject to a known or verified image of the subject to identify the subject with at least about 99% accuracy. In some embodiments, tracking a position of the nose or face of the subject relative to the camera, wherein tracking the position of the nose or face of the subject comprises extrapolating an angle of the nose based upon the at least two points wherein one or more points correspond to a nose bridge and a tip of a nose of the subject.

In some embodiments, the one or more images includes a side profile of the subject. In some embodiments, the side profile of the subject is provided to the machine learning algorithm. In some embodiments, the side profile is used to calculate the nose angle of the subject. In some embodiments the nose angle is calculated using formula 1 by comparing a depth at a middle of the nose z1 and a depth at top of nose z2, where (x1, y1, z1) and (x2, y2, z2) are the Cartesian-coordinates of the two points, respectively.

$${tan}^{- 1} ( \frac{\vert {z}_{2} - {z}_{1} \vert}{\vert {y}_{2} - {y}_{1} \vert} ) * \frac{180}{π} \qquad ( 1 )$$

In some embodiments, the nose angle of the subject is measured by comparing the tip of the nose of the subject with a depth of the nose of the subject at a top of the nose bridge of the nose of the subject. In some embodiments, the accuracy of measuring the nose angle of the subject is within ±2°. In some embodiments, the accuracy of measuring the nose angle of the subject is within +3°. In some embodiments, the accuracy of measuring the nose angle of the subject is within ±4°. In some embodiments, the accuracy of measuring the nose angle of the subject is within ±5°. In some embodiments, the accuracy of measuring the nose angle of the subject is within ±6°. In some embodiments, the accuracy of measuring the nose angle of the subject is within ±7°. In some embodiments, the accuracy of measuring the nose angle of the subject is within ±8°. In some embodiments, the accuracy of measuring the nose angle of the subject is within ±9°. In some embodiments, the accuracy of measuring the nose angle of the subject is within ±10°. In some embodiments, the accuracy of measuring the nose angle of the subject is within ±11°. In some embodiments, the accuracy of measuring the nose angle of the subject is within ±12°. In some embodiments, the accuracy of measuring the nose angle of the subject is within ±13°. In some embodiments, the accuracy of measuring the nose angle of the subject is within ±14°. In some embodiments, the accuracy of measuring the nose angle of the subject is within ±15°.

In some embodiments, the marker on the device comprises at least two data points which indicate an alignment state of the device in relation to one or more facial features of a subject. In some embodiments, an alignment state of the device is determined based on the position of the marker on the device relative to one or more facial features of the subject. In some embodiments, an alignment state of the device is determined based on the position of the marker on the device relative to one or more indicia of the subject. In some embodiments, an alignment state of the device is determined based on the position of the marker on the device relative to one or more facial features of the subject and the one or more indicia of the subject. In some embodiments, a position of the device in the orifice of the subject is captured and compared relative to a database of videos or images. In some embodiments, the videos or images comprises a device in the orifice of the subject. In some embodiments, a comparison of the position of the device in the orifice of the subject to videos or images of a device in the orifice of the subject indicates a proper alignment state of the device. In some embodiments, the comparison of the captured position of the device in the orifice of the subject to videos or images of a device in the orifice of the subject indicates an improper alignment state of the device. In some embodiments, the position of the device relative to the subject is determined by applying a bounding box around the marker. In some embodiments, the position of the device is determined based upon a pitch or a yaw of the device. In some embodiments, the pitch or the yaw of the device is calculated based upon the shape of the bounding box. In some embodiments, a position of a housing compared to the position of the first marker is used to calculate a pitch of the device. In some embodiments, the pitch or the yaw of the device is calculated based upon a measurement of the marker of the device to a projection of a plane defined by the measurement of the marker of the device. In some embodiments, the projection of the plane defined by the known measurement of the marker is captured as a 3D object projected into a 2D space. In some embodiments, the pitch of the device is calculated based on the projection onto the plane using formula 2, wherein the observed height and observed width are known values of the device.

$${cos}^{- 1} ( \frac{observed height}{observed width} ) * \frac{180}{π} \qquad ( 2 )$$

In some embodiments, the camera comprises a depth camera, wherein the pitch of the device is calculated based upon the projection onto the plane using formula 3, wherein A is a height of the fiducial marker and wherein z1 and z2 are depth of the bounding box.

$${sin}^{- 1} ( \frac{\vert {z}_{2} - {z}_{1} \vert}{λ} ) * \frac{180}{π} \qquad ( 3 )$$

In some embodiments, the pitch of the device is calculated with an accuracy of ±2° when the pitch is greater than 20°. In some embodiments, the pitch of the device is calculated with an accuracy of ±3° when the pitch is greater than 20°. In some embodiments, the pitch of the device is calculated with an accuracy of ±4° when the pitch is greater than 20°. In some embodiments, the pitch of the device is calculated with an accuracy of ±5° when the pitch is greater than 20°. In some embodiments, the pitch of the device is calculated with an accuracy of ±6° when the pitch is greater than 20°. In some embodiments, the pitch of the device is calculated with an accuracy of ±7° when the pitch is greater than 20°. In some embodiments, the pitch of the device is calculated with an accuracy of ±8° when the pitch is greater than 20°. In some embodiments, the pitch of the device is calculated with an accuracy of ±9° when the pitch is greater than 20°. In some embodiments, the pitch of the device is calculated with an accuracy of ±10° when the pitch is greater than 20°. In some embodiments, the pitch of the device is calculated with an accuracy of ±11° when the pitch is greater than 20°. In some embodiments, the pitch of the device is calculated with an accuracy of ±12° when the pitch is greater than 20°. In some embodiments, the pitch of the device is calculated with an accuracy of ±13° when the pitch is greater than 20°. In some embodiments, the pitch of the device is calculated with an accuracy of ±14° when the pitch is greater than 20°. In some embodiments, the pitch of the device is calculated with an accuracy of ±15° when the pitch is greater than 20°. In some embodiments, the pitch of the device is calculated with an accuracy of ±10° when the pitch is 20° or is lower than 20°. In some embodiments, the pitch of the device is calculated with an accuracy of ±11° when the pitch is 20° or is lower than 20°. In some embodiments, the pitch of the device is calculated with an accuracy of ±12° when the pitch is 20° or is lower than 20°. In some embodiments, the pitch of the device is calculated with an accuracy of ±13° when the pitch is 20° or is lower than 20°. In some embodiments, the pitch of the device is calculated with an accuracy of ±14° when the pitch is 20° or is lower than 20°. In some embodiments, the pitch of the device is calculated with an accuracy of ±15° when the pitch is 20° or is lower than 20°. In some embodiments, the accuracy of the pitch relative to the nose of the subject is calculated with formula 4:

$${σ}_{combined} = \sqrt{{σ}_{device}^{2} + {σ}_{nose}^{2}} \qquad ( 4 )$$

where σ_{device} is the accuracy of the pitch of the device and σₙₒₛₑ is the accuracy of the pitch of the nose. In some embodiments, the yaw of the device is calculated based upon a vertical edge of the bounding box. In some embodiments, the yaw of the device is calculated based upon the projection of the plane using formula 5, wherein x1 is the horizontal coordinate at a top corner (e.g., top left corner or top right corner) of the marker, x2 is a horizontal coordinate at a bottom corner of the marker (e.g., bottom left corner or top right corner), y1 is a vertical coordinate at a top corner of the marker, and y2 is a vertical coordinate at a bottom corner of the marker. In some embodiments, the top corner and bottom corner of the marker are on the same side of the marker (e.g., left or right).

$${tan}^{- 1} ( \frac{{x}_{1} - {x}_{2}}{{y}_{1} - {y}_{2}} ) * \frac{180}{π} \qquad ( 5 )$$

In some embodiments, the yaw is calculated with an accuracy of ±3°. In some embodiments, the yaw is calculated with an accuracy of ±4°. In some embodiments, the yaw is calculated with an accuracy of ±5°. In some embodiments, the yaw is calculated with an accuracy of ±6°. In some embodiments, the yaw is calculated with an accuracy of ±7°. In some embodiments, the yaw is calculated with an accuracy of ±8°. In some embodiments, the yaw is calculated with an accuracy of ±9°. In some embodiments, the yaw is calculated with an accuracy of ±10°. In some embodiments, the yaw is calculated with an accuracy of ±11°. In some embodiments, the yaw is calculated with an accuracy of ±12°. In some embodiments, the yaw is calculated with an accuracy of ±13°. In some embodiments, the yaw is calculated with an accuracy of ±14°. In some embodiments, the yaw is calculated with an accuracy of ±15°. In some embodiments, the accuracy of the yaw relative to the subject's nose is calculated with formula 4. In some embodiments, if the device is in an improper configuration state, feedback is provided to a computing device of the subject as to the configuration state of the device, wherein the feedback indicates that the configuration state of the device needs to be adjusted. In some embodiments, feedback is provided to the subject as to the alignment state of the device, wherein the subject is directed to adjust the alignment state of the device if it is determined the device is in an improper alignment state. In some embodiments, feedback is provided as to the alignment state of the device, wherein the subject is provided an indication of a proper alignment state of the device prior to actuating the device. In some embodiments, the position of the one or more subject facial features relative to the camera are continuously tracked and continuously calculated.

In some embodiments, identifying the subject using one or more subject indicia comprises applying a machine learning algorithm to identify the subject. In some embodiments, the machine learning algorithm is a facial recognition algorithm. In some embodiments, the one or more subject indicia comprises one or more facial features of the subject. In some embodiments, identifying the subject comprises matching the one or more facial features of the subject to a data file comprising data corresponding to one or more facial features of the subject. In some embodiments, identifying the subject comprises matching the one or more facial features of the subject to a data file comprising a verified image of the subject. In some embodiments, the data file is stored on the network. In some embodiments, the data file is stored on the database of the computing device. In some embodiments, the data file is transferred from the network to the database of the computing device. In some embodiments, the subject information comprises the data file. In some embodiments, the machine learning algorithm isolates a face within an individual frame of a captured video from the camera for analysis. In some embodiments, identifying the subject using one or more subject indicia comprises applying a facial recognition algorithm to identify the subject by matching the one or more subject indicia to a known facial scan of the subject. In some embodiments, identifying the subject comprises instructing the subject to position a face of the subject within a target field of view of the camera. In some embodiments, identifying the subject comprises indicating to the subject when the face of the subject is correctly positioned within the target field of view of the camera. In some embodiments, the subject is continuously identified during performance of the method disclosed herein. In some embodiments, the subject is continuously identified at multiple time points during performance of the method disclosed herein. In some embodiments, the subject is continuously identified during the use of the device, the subject is continuously verified during use of the device, the alignment state of the device continuously identified, the alignment state of the device continuously verified, the configuration state of the device continuously identified, the configuration state of the device continuously verified, or any combination thereof. In some embodiments, the subject is re-identified if the subject is determined to leave the field of view of the camera during performance of the method disclosed herein.

In some embodiments, the device is identified by scanning and identifying indica located on the device with the computing device. In some embodiments, the device is verified as corresponding to a prescription by matching the device to a data file comprising data corresponding to a prescription. In some embodiments, the device is verified as corresponding to a prescription belonging to the subject based upon a positive device identification, a positive prescription identification, a positive subject identification, or any combination thereof. In some embodiments, an impediment is identified while identifying the subject or verifying the device, and providing feedback to correct the impediment. In some embodiments, the impediment comprises the subject or the device being positioned too far away from the camera, an improper lighting state, a physical obstruction in view of the camera, an improper subject head angle, a subject position out of the field of view of the camera, or any combination thereof. In some embodiments, the improper lighting state comprises glare reflection. In some embodiments, a configuration state of the device is continuously identified during performance of the method disclosed herein. In some embodiments, an alignment state of the device is continuously identified during performance of the method disclosed herein. In some embodiments, a configuration state of the device is continuously identified at multiple time points during performance of the method disclosed herein. In some embodiments, an alignment state of the device is continuously identified at multiple time points during performance of the method disclosed herein. In some embodiments, a position of the device is continuously identified during the actuating of the device, verifying actuation of the device, the determining the alignment state of the device, or any combination thereof.

In some embodiments, a configuration state of the device is verified based upon the position of the one or more submarkers of the marker. In some embodiments, the subject is prompted to actuate the device. In some embodiments where the device 120 is not configured to communicate with computing device 110, the subject is prompted to actuate the device upon verifying a proper configuration state of the device, as described above. In some embodiments, the subject is prompted to re-actuate the device. In some embodiments where the device 120 is configured to communicate with computing device 110, the device comprises a securing mechanism preventing actuation of the device. In those embodiments, the securing mechanism allows actuation of the device after the subject, the device, the device being in the first configuration state, and the subject being in need of a compound are verified. In those embodiments, the securing mechanism allows actuation of the device after the subject, the device, the device being in the first configuration state, and the subject being in need of a compound are continuously verified. In those embodiments, the securing mechanism is released upon verifying a positive subject identification, a positive prescription identification, a proper alignment state of the device, or any combinations thereof. In those embodiments, the securing mechanism is released upon a signal transmitted from the computing device to the device. In some embodiments, verifying delivery of the composition to the subject comprises maintaining a continuous positive subject identification throughout the monitoring of the device. In some embodiments, a negative subject identification and a positive prescription identification result in not releasing the securing mechanism and preventing actuation of the device.

In some embodiments, a report is generated. In some embodiments, the report provides one or more images of the method undertaken as disclosed herein. In some embodiments, the report provides one or more videos of the method undertaken as disclosed herein. In some embodiments, the report provides an incorrect dose alert. In some embodiments, the report provides a tampering alert. In some embodiments, the report provides details regarding actuation of the device not administering the compound. For example, details regarding actuation of the device include when the device was actuated, if the subject was the correct subject, if the device was actuated at a prescribed time (e.g., according to a prescription), and if the device was in the correct position with respect to the subject. While some details regarding actuation of the device are listed, these details are exemplary and other details may be used.

In some embodiments, the data file comprising data corresponding to one or more facial features of the subject, the data file comprising a verified image of the subject, the data file comprising data corresponding to a prescription are a part of subject information. In some embodiments, the data file comprising data corresponding to one or more facial features of the subject, the data file comprising a verified image of the subject, the data file comprising data corresponding to a prescription are stored locally on the computing device. In some embodiments, the data file comprising data corresponding to one or more facial features of the subject, the data file comprising a verified image of the subject, the data file comprising data corresponding to a prescription are stored remotely from the computing device. In some embodiments, the data file comprising data corresponding to one or more facial features of the subject, the data file comprising a verified image of the subject, the data file comprising data corresponding to a prescription are stored remotely on a network. In some embodiments, one or more images of the subject are captured during the actuating of the device. In some embodiments, one or more images of the subject are captured during the identifying the subject. The one or more images of the subject can be used post-delivery of the composition, for example, to detect response to the composition. In some embodiments, the first fiducial marker comprises a bar code, a QR code, or other camera readable code. In some embodiments, the fiducial marker comprises a bar code, a QR code, or other camera readable code comprising known positioning data. In some embodiments, the fiducial marker comprises an ArUco marker.

In some embodiments, the machine learning algorithm is updated based upon a captured image or video of the user. In some embodiments, an object or obstruction is detected within a reference frame. In some embodiments, objects or obstructions are identified within the reference frame based upon a shape, a contour, a color, or a texture of the object or obstruction. In some embodiments, a position of the object or obstruction is localized within a reference frame. In some embodiments, a distance between the camera and the subject and/or device is measured using a relative scale. In some embodiments, the relative scale increases the accuracy of the measurement. In some embodiments, patient identification, device identification, detection of device configuration state, detection of device alignment state, detection of device actuation, and/or corrective guidance are performed simultaneously.

In some embodiments, a data report is generated comprising subject identity, device serial number, drug identity, date, time, location, a video of performance of the method disclosed herein, a photo of performance of the method disclosed herein, an actuation event, face position at actuation, device position at actuation, an actuation confirmation, an incorrect dose alert, a tampering alert, details of the actuation of the device as described above, or any combination thereof. In some embodiments, storing a data in a computer readable medium following performance of any part of the method disclosed herein. In some embodiments, the data comprises image or video data. In some embodiments, the image or video data is stored as a JSON object structure. In some embodiments, the JSON object structure comprises keys mapping the configuration state of the device, the alignment state of the device, the identifying the subject, the identifying the device, the actuating the device, or any combination thereof relative to a state of performance of the method disclosed herein.

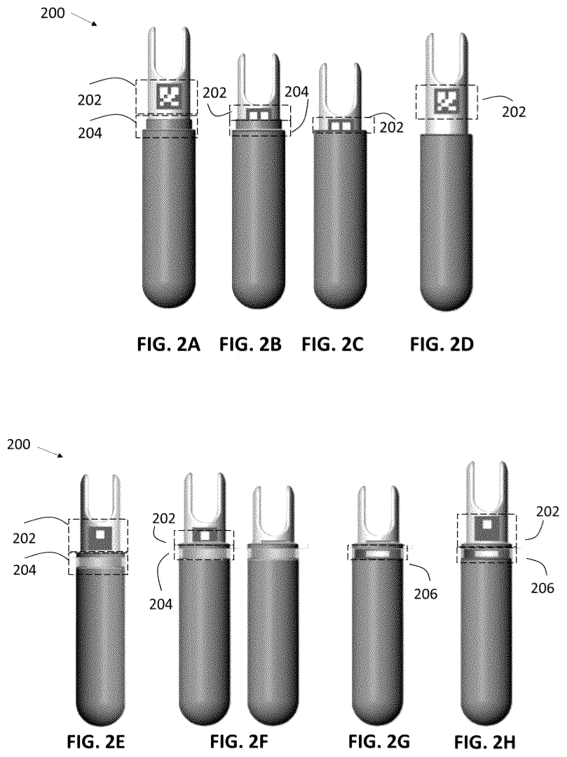

FIGS. 2A-2D depicts a non-limiting example of device 200. In some embodiments, device 200 is the same as device 120 of FIG. 1. In each of FIGS. 2A-2D, the device comprises a marker that may be entirely visible (e.g., the proper configuration state as shown in FIG. 2A), partially concealed (e.g., the unactuated configuration state and actuated configuration state as shown in FIGS. 2B and 2C, respectively), or entirely concealed. FIG. 2A depicts a non-limiting example of device 200 in a fully extended orientation. In this depicted embodiment, the entirety of marker 202 and housing 204 are not concealed. In some embodiments, the configuration state of FIG. 2A, wherein the entirety of marker 202 and housing 204 are not concealed, is a proper configuration state (e.g., the first configuration state as described with respect to FIG. 1). FIG. 2B depicts a non-limiting example of device 200 in a half-extended orientation. In this depicted embodiment, the top portion of marker 202 is not concealed, while the bottom portion of marker is concealed, and the housing 204 is not concealed. In some embodiments, the configuration state of FIG. 2B, wherein the top portion of marker 202 and housing 204 are not concealed, is an active state (e.g., the second configuration state as described with respect to FIG. 1). FIG. 2C depicts a non-limiting example of device 200 in a non-extended orientation. In this depicted embodiment, the top portion of marker 202 is not concealed while the bottom portion of marker 202 and housing 204 are concealed. In some embodiments, the configuration state of FIG. 2C, wherein the top portion of marker 202 is not concealed and housing 204 is concealed, is an inactive state (e.g., the third configuration state as described with respect to FIG. 2). In another non-limiting example of device 200, the entirety of marker 202 (e.g., the top portion and the bottom portion of marker) and housing 204 are concealed. FIG. 2D depicts a non-limiting example of device 200 in an extended orientation. In this depicted embodiment, the marker 202 is not concealed, while the housing 204 is concealed. In some embodiments, the configuration state of FIG. 2D is an inactive state (e.g., the fourth configuration state as described with respect to FIG. 1). In some embodiments, the configuration state with the entirety of the marker and housing concealed is an inactive state (e.g., because the device has been actuated).

FIGS. 2E-2H depict another non-limiting example of device 200, comprising a marker 202, a first housing 204, and a second housing 206. In some embodiments, device 200 is the same as device 120 of FIG. 1. In each of FIGS. 2E-2H, the device comprises a marker that may be entirely visible (e.g., the proper configuration state as shown in FIG. 2E and Actuation Confirmation/Not Use Ready configuration state as shown in FIG. 2H), partially concealed (e.g., the Actuating configuration state as shown in FIG. 2F), or entirely concealed (e,g the Actuated configuration state as shown in FIG. 2G). FIG. 2E depicts a non-limiting example of device 200 in a fully extended orientation. In this depicted embodiment, the entirety of marker 202 and first housing 204 are not concealed, while the second housing 206 is concealed. In some embodiments, the configuration state of FIG. 2E, wherein the entirety of marker 202 and first housing 204 are not concealed while the second housing 206 is concealed, is a proper configuration state (e.g., the first configuration state as described with respect to FIG. 1). FIG. 2F depicts a non-limiting example of device 200 where the bottom portion of marker is concealed, the top portion or marker 202 is visible or concealed, the first housing 204 is not concealed, and the second housing 206 is concealed. In some embodiments, the configuration state of FIG. 2F is an active state (e.g., the second configuration state as described with respect to FIG. 1). FIG. 2G depicts a non-limiting example of device 200 in a non-extended orientation. In this depicted embodiment, the marker 202 and the first housing 204 are concealed, while the second housing 206 is not concealed. In some embodiments, the configuration state of FIG. 2G, wherein the first housing 204 is concealed but the second housing is not concealed, is an inactive state (e.g., the third configuration state as described with respect to FIG. 1). FIG. 2H depicts a non-limiting example of device 200 in an extended orientation. In this depicted embodiment, the marker 202 and the second housing 206 are not concealed, while the first housing 204 is concealed. In some embodiments, the configuration state of FIG. 2H is an inactive state (e.g., the fourth configuration state as described with respect to FIG. 1). In some embodiments, the configuration state with the entirety of the marker and first housing concealed is an inactive state (e.g., because the device has been actuated).

In some embodiments, the housing comprises a first housing marker. In some embodiments, the first housing marker comprises an annular ring. In some embodiments, the first housing marker comprises a color distinct from the housing. In some embodiments, the second housing comprises a second housing marker. In some embodiments, the second housing marker comprises a color distinct from the first housing marker.

In some embodiments, device 200 is monitored by another device (e.g., computing device 110 of FIG. 1). In some embodiments, device 200 is monitored for the configuration state. In some embodiments, the configuration state of device 200 is recognized by a computing device (e.g., by recognition component 112 of computing device 110 of FIG. 1) and verified by the computing device (e.g., by confirmation component 114 of computing device 110 of FIG. 1). In some embodiments, the configuration state of device 200 is recognized by a computing device (e.g., by recognition component 112 of computing device 110 of FIG. 1) and verified by a computing device (e.g., by confirmation component 114 of computing device 110 of FIG. 1) as being in a second (e.g., unactuated) configuration state (e.g., the configuration state depicted in FIG. 2B). In some embodiments, the second configuration state (e.g., the configuration state depicted in FIG. 2B) is an active configuration state. In some embodiments, the configuration state of device 200 is recognized by a computing device (e.g., by recognition component 112 of computing device of FIG. 1) and verified by a component (e.g., by confirmation component 114 of computing device FIG. 1) as being in a first (e.g., proper) configuration state (e.g., the configuration state depicted in FIG. 2A). In some embodiments, the first configuration state (e.g., the configuration state depicted in FIG. 2A) is an inactive and actuatable configuration state. In some embodiments, the configuration state of device 200 is recognized by a computing device (e.g., by recognition component 112 of computing device 110 of FIG. 1) and verified by the computing device (e.g., by confirmation component 114 of computing device 110 FIG. 1) as being in a third configuration state (e.g., the configuration state depicted in FIG. 2C). In some embodiments, the third configuration state (e.g., the configuration state as depicted in FIG. 2C) is an inactive and non-actuatable configuration state. In some embodiments, the configuration state of device 200 is recognized by a computing device (e.g., by recognition component 112 of computing device 110 of FIG. 1) and verified by the computing device (e.g., by confirmation component 114 of computing device 110 FIG. 1) as being in a fourth configuration state. In some embodiments, the fourth configuration state is an inactive and non-actuatable configuration state. In some embodiments, the fourth configuration state of the device is a configuration state in which the marker is not concealed but the housing is concealed, indicating that the device has been actuated. In some embodiments, the fourth configuration state confirms actuation. In some embodiments, the fourth configuration state indicates a device is not ready for actuation, for example, because the device is not loaded or the device has been used.

In some embodiments, each configuration state of the device can indicate a position of the device with respect to the target region of the subject. In some embodiments, the position of the device is only determined when the device is in the first configuration state. In some embodiments, the target region is an orifice of a subject. In some embodiments, the target region is a mouth of a subject. In some embodiments, the target region is the nasal cavity of a subject. In some embodiments, the target region is an olfactory region of a subject. In some embodiments, the target region is a columella of the subject. In some embodiments, the marker is not concealed by the housing and the housing is not concealed in a first configuration state. In some embodiments, the marker is partly concealed by the housing and the housing is concealed in a third configuration.

In some embodiments, the configuration state of the device being the first or second configuration states indicates the device can be actuated. In some embodiments, a first submarker (e.g., the bottom portion of marker 202 as described with respect to FIGS. 2B-2C) of the one or more submarkers is concealed by the housing and a second submarker (e.g., the top portion of marker 202 as described with respect to FIGS. 2B-2C) of the one or more submarkers is not concealed by the housing and the housing is not concealed in the second configuration state. In some embodiments, the first submarker of the one or more submarkers is not concealed by the housing and a second submarker of the one or more submarkers is not concealed by the housing and the housing is not concealed in the first configuration state. In some embodiments, the first submarker of the one or more submarkers is not concealed by the housing and a second submarker of the one or more submarkers is concealed by the housing and the housing is concealed in the third configuration state. In some embodiments, the marker is not concealed by the housing and the housing is not concealed in the first configuration state (e.g. FIG. 2E). In some embodiments, the marker is at least partially concealed by the housing, the housing is not concealed, and the second housing is concealed in the second configuration state (e.g. FIG. 2F). In some embodiments, the marker and the housing are concealed, and the second housing is not concealed in the third configuration state (e.g. FIG. 2G). In some embodiments, the marker is not concealed, the housing is concealed, and the second housing is not concealed in the fourth configuration state (e.g. FIG. 2H).

In some embodiments, the device is partially inserted into an orifice of the subject. In some embodiments, the orifice is the nasal cavity of the subject. In some embodiments, the device is partially inserted into the nasal cavity of the subject. In some embodiments, the partial insertion of the device into the orifice of a subject causes the device to transition from the first configuration state to the second configuration state. In some embodiments, the device is partially inserted into the orifice of the subject at a prescribed depth and angle. In some embodiments, an alignment state of the device and the subject is based upon the position of the fiducial marker. In some embodiments, the alignment state of the device and the subject is based upon a position of the fiducial marker relative to one or more subject facial features. In some embodiments, the alignment state of the device and the subject is based upon a position of the fiducial marker relative to one or more criteria. In some embodiments, the alignment state of the device and the subject is based upon a position of the fiducial marker relative to one or more subject facial features and the one or more criteria. In some embodiments, the position comprises an alignment state of the device. In some embodiments, a recommendation is provided to adjust the alignment state of the device. In some embodiments, the recommendation to adjust the alignment state of the device is provided if it is determined the alignment state is improper relative to one or more facial features of the subject.

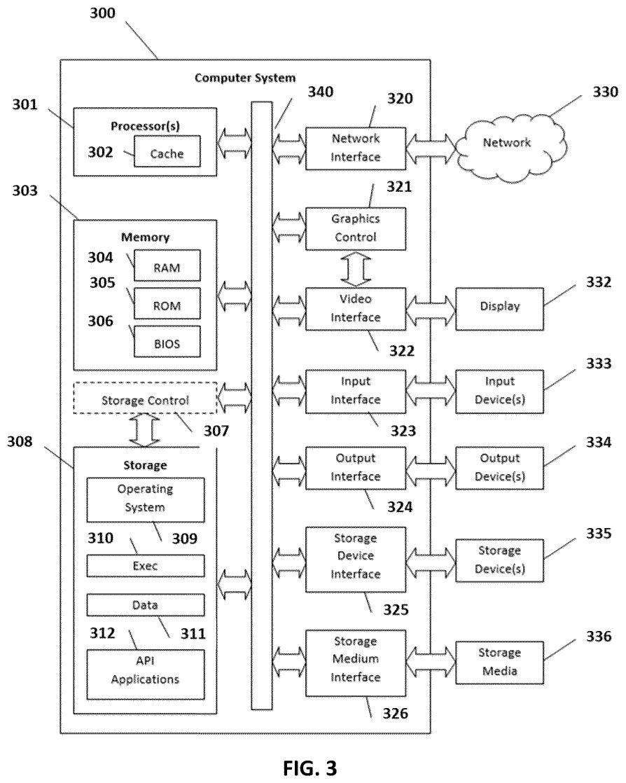

Referring to FIG. 3, a block diagram is shown depicting an exemplary machine that includes a computer system 300 (e.g., a processing or computing system) within which a set of instructions can execute for causing a device to perform or execute any one or more of the aspects and/or methodologies for static code scheduling of the present disclosure. The components in FIG. 3 are examples only and do not limit the scope of use or functionality of any hardware, software, embedded logic component, or a combination of two or more such components implementing particular embodiments.

Computer system 300 may include one or more processors 301, a memory 303, and a storage 308 that communicate with each other, and with other components, via a bus 340. The bus 340 may also link a display 332, one or more input devices 333 (which may, for example, include a keypad, a keyboard, a mouse, a stylus, etc.), one or more output devices 334, one or more storage devices 335, and various tangible storage media 336. All of these elements may interface directly or via one or more interfaces or adaptors to the bus 340. For instance, the various tangible storage media 336 can interface with the bus 340 via storage medium interface 326. Computer system 300 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 300 includes one or more processor(s) 301 (e.g., central processing units (CPUs), general purpose graphics processing units (GPGPUs), or quantum processing units (QPUs)) that carry out functions. Processor(s) 301 optionally contains a cache memory unit 302 for temporary local storage of instructions, data, or computer addresses. Processor(s) 301 are configured to assist in execution of computer readable instructions. Computer system 300 may provide functionality for the components depicted in FIG. 3 as a result of the processor(s) 301 executing non-transitory, processor-executable instructions embodied in one or more tangible computer-readable storage media, such as memory 303, storage 308, storage devices 335, and/or storage medium 336. The computer-readable media may store software that implements particular embodiments, and processor(s) 301 may execute the software. Memory 303 may read the software from one or more other computer-readable media (such as mass storage device(s) 335, 336) or from one or more other sources through a suitable interface, such as network interface 320. The software may cause processor(s) 301 to carry out one or more processes or one or more steps of one or more processes described or illustrated herein. Carrying out such processes or steps may include defining data structures stored in memory 303 and modifying the data structures as directed by the software.

The memory 303 may include various components (e.g., machine readable media) including, but not limited to, a random access memory component (e.g., RAM 304) (e.g., static RAM (SRAM), dynamic RAM (DRAM), ferroelectric random access memory (FRAM), phase-change random access memory (PRAM), etc.), a read-only memory component (e.g., ROM 305), and any combinations thereof. ROM 305 may act to communicate data and instructions unidirectionally to processor(s) 301, and RAM 304 may act to communicate data and instructions bidirectionally with processor(s) 301. ROM 305 and RAM 304 may include any suitable tangible computer-readable media described below. In one example, a basic input/output system 306 (BIOS), including basic routines that help to transfer information between elements within computer system 300, such as during start-up, may be stored in the memory 303.

Fixed storage 308 is connected bidirectionally to processor(s) 301, optionally through storage control unit 307. Fixed storage 308 provides additional data storage capacity and may also include any suitable tangible computer-readable media described herein. Storage 308 may be used to store operating system 309, executable(s) 310, data 311, applications 312 (application programs), and the like. Storage 308 can also include an optical disk drive, a solid-state memory device (e.g., flash-based systems), or a combination of any of the above. Information in storage 308 may, in appropriate cases, be incorporated as virtual memory in memory 303.

In one example, storage device(s) 335 may be removably interfaced with computer system 300 (e.g., via an external port connector (not shown)) via a storage device interface 325. Particularly, storage device(s) 335 and an associated machine-readable medium may provide non-volatile and/or volatile storage of machine-readable instructions, data structures, program modules, and/or other data for the computer system 300. In one example, software may reside, completely or partially, within a machine-readable medium on storage device(s) 335. In another example, software may reside, completely or partially, within processor(s) 301.

Bus 340 connects a wide variety of subsystems. Herein, reference to a bus may encompass one or more digital signal lines serving a common function, where appropriate. Bus 340 may be any of several types of bus structures including, but not limited to, a memory bus, a memory controller, a peripheral bus, a local bus, and any combinations thereof, using any of a variety of bus architectures. As an example, and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 300 may also include an input device 333. In one example, a user of computer system 300 may enter commands and/or other information into computer system 300 via input device(s) 333. Examples of an input device(s) 333 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 333 may be interfaced to bus 340 via any of a variety of input interfaces 323 (e.g., input interface 323) including, but not limited to, serial, parallel, game port, USB, FIREWIRE, THUNDERBOLT, or any combination of the above.

In particular embodiments, when computer system 300 is connected to network 330, computer system 300 may communicate with other devices, specifically mobile devices and enterprise systems, distributed computing systems, cloud storage systems, cloud computing systems, and the like, connected to network 330. Communications to and from computer system 300 may be sent through network interface 320. For example, network interface 320 may receive incoming communications (such as requests or responses from other devices) in the form of one or more packets (such as Internet Protocol (IP) packets) from network 330, and computer system 300 may store the incoming communications in memory 303 for processing. Computer system 300 may similarly store outgoing communications (such as requests or responses to other devices) in the form of one or more packets in memory 303 and communicated to network 330 from network interface 320. Processor(s) 301 may access these communication packets stored in memory 303 for processing.

Examples of the network interface 320 include, but are not limited to, a network interface card, a modem, and any combination thereof. Examples of a network 330 or network segment 330 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 330, may employ a wired and/or a wireless mode of communication. In general, any network topology may be used.

Information and data can be displayed through a display 332. Examples of a display 332 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 332 can interface to the processor(s) 301, memory 303, and fixed storage 308, as well as other devices, such as input device(s) 333, via the bus 340. The display 332 is linked to the bus 340 via a video interface 322, and transport of data between the display 332 and the bus 340 can be controlled via the graphics control 321. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 332, computer system 300 may include one or more other peripheral output devices 334 including, but not limited to, an audio speaker, a printer, a storage device, and any combinations thereof. Such peripheral output devices may be connected to the bus 340 via an output interface 324. Examples of an output interface 324 include, but are not limited to, a serial port, a parallel connection, a USB port, a FIREWIRE port, a THUNDERBOLT port, and any combinations thereof.

In addition or as an alternative, computer system 300 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TVR, Google Chromecast®, Amazon Fire®, and Samsung® HomeSync®. Those of skill in the art will also recognize that suitable video game console operating systems include, by way of non-limiting examples, Sony® PS3®, Sony® PS4®, Sony® PS5®, Microsoft® Xbox 360®, Microsoft® Xbox One, Microsoft® Xbox Series X, Microsoft® Xbox Series S, Nintendo® Wii®, Nintendo® Wii U®, Nintendo® Switch™, and Ouya®.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and UnityR.

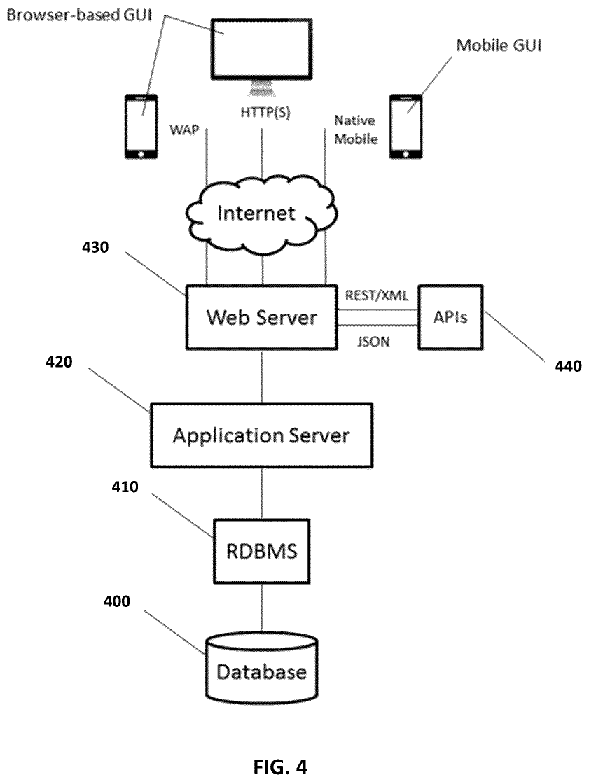

Referring to FIG. 4, in a particular embodiment, an application provision system comprises one or more databases 400 accessed by a relational database management system (RDBMS) 410. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application server 420 (such as Java servers, .NET servers, PHP servers, and the like) and one or more web servers 430 (such as Apache, IIS, GWS and the like). The web server(s) optionally expose one or more web services via app application programming interfaces (APIs) 440. Via a network, such as the Internet, the system provides browser-based and/or mobile native user interfaces.

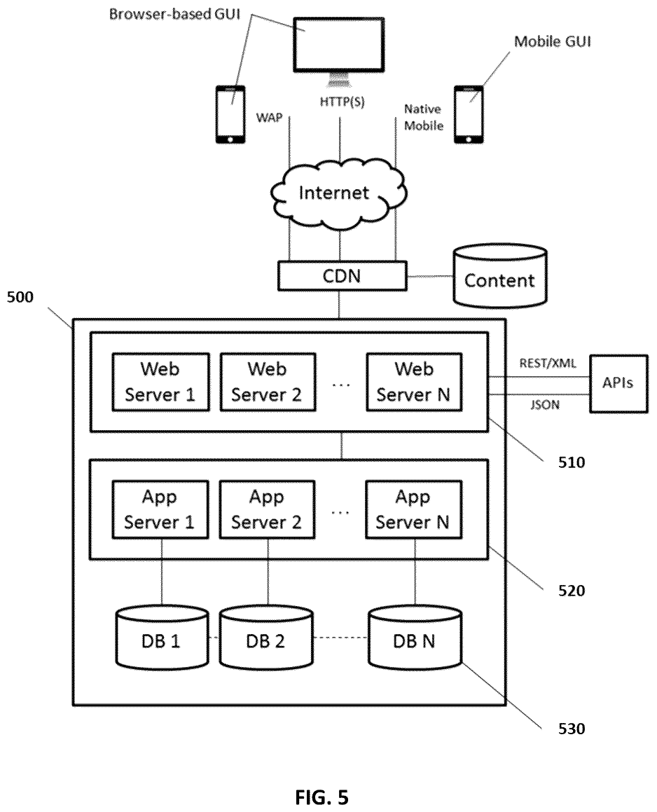

Referring to FIG. 5, in a particular embodiment, an application provision system alternatively has a distributed, cloud-based architecture 500 and comprises elastically load balanced, auto-scaling web server resources 510 and application server resources 520 as well as synchronously replicated databases 530.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, Rails, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable compiled applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of subject, device, and composition information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### Data Transmission

The subject matter described herein, including methods for recognizing and verifying a subject, a device, and a composition may be configured to be performed in one or more facilities at one or more locations. Facility locations are not limited by country and include any country or territory. In some instances, one or more steps are performed in a different country than another step of the method. In some embodiments, one or more method steps involving a computer system are performed in a different country than another step of the methods provided herein. In some embodiments, data processing and storage are performed in a different country or location than one or more steps of the methods described herein. In some embodiments, one or more products or data are transferred from one or more of the facilities to one or more different facilities for analysis or further analysis. Data includes, but is not limited to, information regarding the stratification of a subject, and any data produced by the methods disclosed herein. In some embodiments of the methods and systems described herein, the subject information is compiled, and a subsequent data transmission step will transmit or store the subject information.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as subject, device, or composition information, performed in one facility in a particular location and corresponding data transmitted to another location.

### Business Methods Utilizing a Computer

The methods described herein may utilize one or more computers. The computer may be used for managing customer and subject information such as device or composition information, database management, storing data, billing, marketing, storing subject information, or a combination thereof. The computer may include a monitor or other user interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to a subject, a guardian of the subject, a health care provider (e.g., doctor) or a health care manager.

The entity entering or reviewing information into a database for the purpose of one or more of the following: inventory tracking, order tracking, customer management, customer service, billing, and sales. Sample information may include, but is not limited to: customer name, unique customer identification, subject associated medical provider, compositions of subject, composition usage of subject, medical history, preliminary diagnosis, suspected diagnosis, or any information suitable for storage in a database.

The database may be accessible by a customer, medical professional, or other third party. Database access may take the form of electronic communication such as a computer or telephone. The database may be accessed through an intermediary such as a customer service representative, business representative, consultant, independent testing center, or medical professional. The availability or degree of database access may change upon payment of a fee for products and services rendered or to be rendered.

### Adaptors:

In some embodiments, a device may be adapted for systems or methods for device detection and medicament delivery verification. This adaption may include the addition of components or markings to provide for better visualization of the device identity, position, configuration, or actuation. In some embodiments, one or more markers such as ArUco codes may be added to the adapted device. In some embodiments, one or more sleeves or movable components may be added to the adapted device. In some embodiments, the device design may be adapted prior to manufacture of the device to include one or more features providing for adaptation. In other embodiments, the adapted device may be marked or modified post manufacturing. In some embodiments, a cap or other component of a device may be replaced with a corresponding adaptor component including one or more markers. In some embodiments, adaptation of the device may be limited to only the addition of one or more markers, without additional adaptation. The markers may be fiducial markers, non-fiducial markers, or a combination thereof.

In some embodiments, components of an adaptor kit are designed with minimal or no impact to device architecture. Adaptation of an existing device may provide for patient compliance tracking supported by simultaneous proof positive identification of who, what, how, and when the drug has been taken. Adapted devices may be used to guide and train users on the correct angle, depth, speed, and time of day, and to improve compliance or adherence (e.g. with a medication regimen).

### Multi-Marker Position Detection:

Detecting and tracking device position in three-dimensional space relative to face datums and position both enables device position guidance and training and enables detection of device insertion in the nose of a subject. In some embodiments use of one or more fiducial markers such as ArUco codes are used to detect the position of the device and allow for position calculations as described herein. In some examples, two or more fiducial markers or ArUco codes enable actuation detection and/or speed detection.

### Full/Partial Actuation Detection:

In some embodiments, detection of full or partial actuation enables confirmation that actuation has occurred. In some embodiments, detection of full or partial actuation enables full or fractional dose detection, offering the potential for a better understanding of the amount of a dose delivered.

For example, full or partial actuation may be detected by calculating the space between two markers positioned at a known distance from each other. In some embodiments, as the device is actuated one or more of the markers will move, thereby reducing the space between the markers until the post-actuation release. If released before full actuation, the system may detect a fractional dose as the space between the markers would not have been reduced to indicate full actuation. While both markers may be fiducial markers, in some embodiments one of the markers is a fiducial marker such as an ArUco code and the other is a non-fiducial marker.

In some embodiments, the method further comprises detecting full or fractional actuation. In some embodiments, detecting full or fractional actuation comprises detecting a distance between the marker and the housing and/or between the marker and the second housing.

### Detection of Speed of Actuation:

Detection of the approximate speed of actuation enables detection of the timing of a delivery. This allows for determination if a delivery is too fast, too slow, or within desired specifications. For example, to detect an approximate speed of actuation, the time from the first marker starts moving to the time full actuation is reached will be calculated. In some embodiments, this time may be about 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 550, 600, 650, 700, 750, 800, 850, 900, 950, 1000, or greater than 1000 ms.

### Methods

### Computer-Implemented Methods

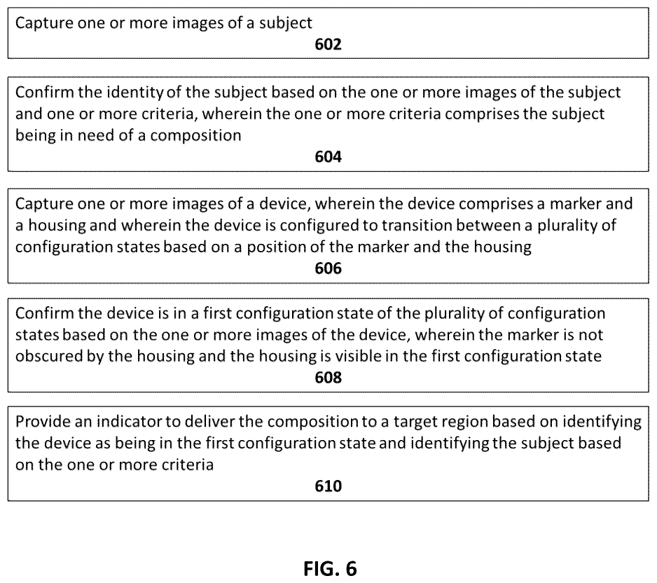

Provided herein are computer-implemented methods for delivering a composition to a target region of a subject. FIG. 6 depicts a non-limiting example of a method for identifying a subject as being in need of a composition and delivering the composition to the subject.

The method begins at 602, where one or more images of a subject are captured. In some embodiments, the one or more images are captured by a computing device (e.g., computing device 110 of FIG. 1). In some embodiments, the one or more images are captured by recognition component 112 of computing device 110 of FIG. 1. In some embodiments, the one or more images are captured by a camera. In some embodiments, the camera is in communication with the computing device (e.g., computing device 110 of FIG. 1). In some embodiments, the camera is connected to the computing device (e.g., computing device 110 of FIG. 1). In some embodiments, recognition component 112 of computing device 110 of FIG. 1 comprises the camera. In some embodiments, the camera is in communication with recognition component 112 of computing device 110 of FIG. 1. In some embodiments, the camera is connected to recognition component 112 of computing device 110 of FIG. 1.

At 604, the identity of the subject is confirmed based on the one or more images captured in 602 as compared to the subject information contained on the network or database and one or more criteria, wherein the one or more criteria comprises the subject being in need of a composition (e.g., medicament). In some embodiments, the subject information is received from a network (e.g., network 130 of FIG. 1). In some embodiments, the subject information is received from a database (e.g., database 116 of computing device 110 of FIG. 1). In some embodiments, the subject is identified using a computing device (e.g., computing device 110 of FIG. 1). In some embodiments, the subject is identified using confirmation component 114 of computing device 110 of FIG. 1. In some embodiments, confirmation component 114 of computing device 110 of FIG. 1 utilizes subject information (e.g., subject information 134 of FIG. 1). In some embodiments, confirmation component 114 of computing device 110 of FIG. 1 utilizes subject information from network 130 of FIG. 1). In some embodiments, confirmation component 114 of computing device 110 of FIG. 1 utilizes subject information from database 116 of FIG. 1.

At 606, one or more images of a device are captured. In this embodiment, the device comprises a marker and a housing. In this embodiment, the device is configured to transition between a plurality of configuration states. In this embodiment, the plurality of configuration states is based on a position of the marker and the housing. In some embodiments where the device is in communication with the computing device, some of the plurality of configuration states are configured to render the device inactive. In some embodiments where the device is in communication with the computing device, one of the plurality of configuration states are configured to activate the device. In some embodiments, where the device is not in communication with the computing device, the computing device may display a recommendation to indicate if the device should be actuated.

At 608, the device is identified as being in a proper configuration state of the plurality of configuration states based on the one or more images captured in 606. In some embodiments, the first configuration state of the plurality of configuration states is identified by the marker not being concealed by the housing and the housing being not concealed as depicted in FIG. 2A. In other embodiments, the device may be identified as being in other configuration states, such as those depicted in FIG. 2B and FIG. 2C.

At 610, an indicator is given to deliver the composition to a target region. In this embodiment, the indicator is given based on identifying the device as being in the first configuration state as in 608 and identifying the subject as in 604. In some embodiments, a second indicator indicating that the medicament should not be delivered is given based on monitoring. In this embodiment, the second indicator may be given if the device position or the configuration state of the device is recognized as improper. In some embodiments, the second indicator causes the device to not deliver the composition. In some embodiments, a third indicator indicating that the medicament should be delivered is given based on monitoring. In some embodiments, the third indicator is given after the second indicator was provided. In this embodiment, the third indicator may be given if the device is moved back into the proper position or configuration state. In some embodiments, the third indicator causes the device to allow for delivery of the composition.

In some embodiments, at 610, a recommendation to deliver the composition is displayed (e.g., on a user interface). In those embodiments, the recommendation to deliver the composition is given based on identifying the device as being in the first configuration state and identifying the subject as the proper subject. In some embodiments, the recommendation to deliver the composition is given based further on a position of the device. In those embodiments, if the position of the device is changed or the configuration state is changed, a second recommendation to not deliver the composition may be given based on the position of the device or the configuration state being changed. In some embodiments, a third recommendation may be given if the position of the device is corrected.

In some embodiments, 602 and 606 are performed at the same time. In some embodiments, 602 is performed prior to 606. In some embodiments, 606 is performed prior to 602. In some embodiments, 604 and 608 are performed at the same time. In some embodiments, 604 is performed prior to 608. In some embodiments, 608 is performed prior to 604. In some embodiments, 602 and 604 are performed prior to 606 and 608. In some embodiments, 606 and 608 are performed prior to 602 and 604. In some embodiments, 602 and 606 are performed prior to 604 and 608. In some embodiments, 604 and 608 are performed prior to 602 and 606.

### Definitions

As used herein, the singular forms “a,” “an,” and “the” include plural references, unless indicated otherwise.

The term “or” is generally used to mean “and/or”, unless it is indicated explicitly to refer to alternatives only. The phrase “and/or” means “either or both” of the elements so conjoined. These terms can convey that any combination is specifically contemplated. It should also be noted that the term “or” is generally employed in its sense including “and/or” unless the content clearly dictates otherwise. These terms can convey that any combination is specifically contemplated. Solely for illustrative purposes, the expression “A, B, and/or C” can mean A individually; B individually; C individually; A and B; B and C; A and C; and A, B, and C.

The term “comprise”, “comprising”, or the like is open-ended and means additional elements or components other than those recited may be present. Other terms such as “include”, “contain”, “have” and the like have similar meaning.

The term “consist of”, “consisting of” or the like is closed-ended and means no additional component is present.

Further, terms of degree such as “substantially”, “about” and “approximately” as used herein mean within an acceptable error range for the particular value, or a reasonable amount of deviation of the modified term, as determined by one of ordinary skill in the art. These terms of degree should be construed as including a deviation of at least ±5% of the modified term if this deviation would not negate the meaning of the word it modifies.

More specifically, the term “about” or “approximately” when used in reference to a particular recited value, means there can be an acceptable variation range, as determined by one of ordinary skill in the art to which this disclosure pertains. The term can mean ±10%, ±5%, ±2%, or ±1%. Where a particular value is recited, it can be understood that the value is modified by the term “about” or “approximately”, unless indicated otherwise. The recitation of numerical ranges includes all numbers and fractions within that range (e.g. 1 to 5 includes 1, 1.5, 2, 2.75, 3, 3.90, 4, and 5).

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### EXAMPLES

The following examples are included for illustrative purposes only and are not intended to limit the scope of the inventive concepts.

### Example 1: Subject in Need of Ketamine Receiving a First Indication

A subject suffering from migraines uses a camera on their smartphone to upload a series of images and videos as instructed by an application. The application analyzes the images and videos and stores them in an accessible network database. The subject also inputs their name and birthdate. A prescription is uploaded to the subject's account by a medical provider. The prescription details the medicine, dosage, and frequency required by the subject. The prescription is for ketamine to be administered intranasally. Each dispense of ketamine through the device holding the ketamine releases 20 mg of ketamine. The prescription frequency is for one intranasal dispense of ketamine every 4 hours as needed for migraines with the possibility of a second dose following 2-15 minutes after the first dose, where the subject should not dispense more than 100 mg of ketamine a day. The subject receives the ketamine in a canister with a pronged end for intranasal dispersion, a QR code, and a housing. The QR code is specific to the subject and their prescription for intranasal ketamine.

One morning the subject awakens with a migraine. The subject opens the application on their smartphone associated with the device containing the ketamine. The subject turns on the camera and follows the prompts on the application. They place their face and the device as-instructed in view of the camera on the smartphone. The smartphone captures one or more images of the subjects face and verifies the identity of the subject, as well as confirming the prescription of the subject and the device to be used by the subject. Upon verification of the identity and the confirmation of the prescription and device, the smartphone displays a prompt indicating that the subject should put the device in the proper position and configuration state for delivering the medicament. The subject properly positions the device while the smartphone is monitoring, and upon recognizing the proper position, the smartphone displays a prompt to actuate the device.

### Example 2: Subject in Need of Ketamine Receives Ketamine from the Device

To begin, the process of Example 1 is performed. The subject then adds pressure to the canister upward to place the device in a second configuration state, and actuates the device, which causes the device to deliver the medicament and move to a third configuration state. Upon delivering the medicament, the smartphone indicates to the subject that the medicament has been delivered and generates a report indicating whether the position of the device was proper, whether the subject was the correct subject, what time the medicament was delivered, and if the medicament was delivered at a time consistent with the subject's prescription.

### Example 3: Subject in Need of Ketamine Receiving a Second Indication

To begin, the process of Example 1 is performed. However, prior to the subject using the device to dispense the ketamine, the device moves away from the nose of the subject and into an improper position. The smartphone is monitoring the entire event and verifies the device is now in an improper position for dispersion of ketamine. Upon verifying the device is in an improper position, the smartphone sends a second indicator to the device indicating that the device should not dispense the ketamine. In response, the device blocks the ketamine from being dispensed.

### Example 4: Subject in Need of Ketamine Receiving a Third Indication

To begin, the process of Example 3 is performed. Upon receiving the second indicator, the subject receives a prompt on the screen of their smartphone for the application informing the subject of the issue that resulted in the second indicator and instructions of how to correct the issue. The subject corrects the position of the device. The smartphone, which has continuously monitored the device and the subject, recognizes that the device is again in the proper position and sends a third indicator to the device that the device can now dispense the ketamine.

### Example 5: Subject not Recognized

A subject with migraines has been prescribed ketamine for treatment through intranasal administration. To avoid overuse or misuse, the subject is only able to dispense the ketamine to themselves as directed in their prescription. While asleep early one morning, the subject's roommate takes their device and smartphone to administer ketamine to themself. The roommate opens the application and follows the prompts, placing their face and the device in view of camera on the smartphone. The application retrieves images of the subject and compares them to the image of the roommate currently in view of the camera. The application cannot verify that the roommate is the correct recipient of the ketamine (e.g., as the subject is the correct recipient) from the device. The application sends an indicator to the device blocking the dispersion of ketamine.

### Example 6: Subject Reached Daily Maximum

A subject in need of ketamine for migraines with a device containing ketamine for treatment of their migraine using an intranasal dosage continues to have migraines and goes to use the device containing the ketamine to dispense the dosage required. The subject has used the device five times today for a total of 100 mg of ketamine received intranasally. The subject opens the application on their smartphone associated with the device containing the ketamine. The subject turns on the camera and follows the prompts on the application. The subject places their face and the device as instructed in view of the camera on the smartphone. The subject then adds pressure to the canister upward to place the device in a second configuration state. Although the subject and device are verified by the application, the application notes that the subject has reached their maximum daily dosage of ketamine and the smartphone sends a second indicator to the device to block the dispersion of ketamine.

### Example 7: Subject Requesting Dosage Too Early

To begin, the process of Example 1 is performed. The subject in need of ketamine for migraines with a device containing ketamine for treatment of their migraine using an intranasal dosage continues to have migraines and goes to use the device containing the ketamine to dispense the dosage required. The subject last received a dose of ketamine 3 hours ago. The subject opens the application on their smartphone associated with the device containing the ketamine. The subject turns on the camera and follows the prompts on the application. They place their face and the device as instructed in view of the camera on the smartphone. The subject then adds pressure to the canister upward to place the device in a second configuration state. Although the subject and device are verified by the application, the application notes that the subject needs to wait 4 hours after the last dosage of ketamine before they can get another dosage and the smartphone sends a second indicator to the device to block the dispersion of ketamine.

### Example 8: Subject in Need of Ketamine Receiving a Second Indication

To begin, the process of Example 1 is performed. However, prior to the subject using the device to dispense the ketamine, the device moves away from the nose of the subject and into an improper position. The smartphone is monitoring the entire event and verifies the device is now in an improper position for dispersion of ketamine. Upon verifying the device is in an improper position, the smartphone displays a prompt that the device is not in the correct position and needs to be put into the correct position. Instead, the subject proceeds with dispensing the ketamine in the improper position. The smartphone generates a report that indicates that while the subject was the correct subject in need of ketamine, the device was in the improper position, and therefore, the delivery of the ketamine was improper.

### Example 9: Subject in Need of Ketamine Receiving a Third Indication

To begin, the process of Example 8 is performed. The subject corrects the position of the device in response to the second indicator. The smartphone, which has continuously monitored the device and the subject, recognizes that the device is again in the proper position and displays a prompt that the device is in the correct position and the device should be actuated. The subject then actuates the device while the device is in the proper position. Upon delivering the ketamine, the smartphone generates a report indicating whether the position of the device was proper, whether the subject was the correct subject, what time the medicament was delivered, and if the medicament was delivered at a time consistent with the subject's prescription.

### Example 10: Subject not Recognized

A subject with migraines has been prescribed ketamine for treatment through intranasal administration. To avoid overuse or misuse, the subject is only able to dispense the ketamine to themselves as directed in their prescription. While asleep early one morning, the subject's roommate takes their device and smartphone to administer ketamine to themself. The roommate opens the application and follows the prompts, placing their face and the device in view of the camera on the smartphone. The application retrieves images of the subject and compares them to the image of the roommate currently in view of the camera. The application cannot verify that the roommate is the correct recipient of the ketamine (e.g., as the subject is the correct recipient) from the device. The smartphone displays a prompt indicating that the roommate is not the correct subject for receiving the ketamine. The roommate, ignoring the prompt, proceeds with actuating the device. After the delivery of the ketamine, the smartphone generates a report indicating that the ketamine was not delivered to the correct subject and that the subject may need another dose to complete their prescription.

### Example 11: Device being Tampered with

A subject with migraines has been prescribed ketamine for treatment through intranasal administration. While asleep early one morning, the subject's roommate takes their device. Expecting that the application will not verify that they are the correct recipient of the ketamine, the roommate attempts to forcefully actuate the device. After several unsuccessful attempts, the roommate leaves the device where they found it. A tamper-evident system of the device is triggered as a result of the roommate's action. The subject continues to use the device as prescribed. When the subject returns the device to the health care provider that prescribed the ketamine, the health care provider determines that the device has been tampered with. The health care provider records the details of the tampering in a database.

### Example 12: Device being Tampered with

A subject with migraines has been prescribed ketamine for treatment through intranasal administration. While asleep early one morning, the subject's roommate takes their device. Expecting that the application will not verify that they are the correct recipient of the ketamine, the roommate attempts to forcefully actuate the device. The roommate successfully extract some ketamine from the device and thereafter leaves the device where they found it. A tamper-evident system of the device is triggered as a result of the roommate's action. A tamper indication system notifies the subject of the tampering and sends details of the event to the health care provider.

### Example 13: Subject Reached Daily Maximum

A subject in need of ketamine for migraines with a device containing ketamine for treatment of their migraine using an intranasal dosage continues to have migraines and goes to use the device containing the ketamine to dispense the dosage required. The subject has used the device five times today for a total of 100 mg of ketamine received intranasally. The subject opens the application on their smartphone associated with the device containing the ketamine. The subject turns on the camera and follows the prompts on the application. The subject places their face and the device as instructed in view of the camera on the smartphone. The subject then adds pressure to the canister upward to place the device in a second configuration state. Although the subject and device are verified by the application, the smartphone notes that the subject has reached their maximum daily dosage of ketamine and the smartphone displays a prompt indicating that the maximum dosage has been reached. The subject, ignoring the prompt, proceeds with actuating the device. After the delivery of the ketamine, the smartphone generates a report that although the correct subject received the ketamine, the dose was administered at a time inconsistent with the prescription (e.g., over the maximum daily dose).

### Example 14: Subject Requesting Dosage Too Early

To begin, the process of Example 1 is performed. The subject in need of ketamine for migraines with a device containing ketamine for treatment of their migraine using an intranasal dosage continues to have migraines and goes to use the device containing the ketamine to dispense the dosage required. The subject last received a dose of ketamine 3 hours ago. The subject opens the application on their smartphone associated with the device containing the ketamine. The subject turns on the camera and follows the prompts on the application. They place their face and the device as instructed in view of the camera on the smartphone. Although the subject and device are verified by the application, the smartphone notes that the subject has received the last dose of ketamine within 3 hours, and another hour of wait time is required before receiving another dose. The smartphone then displays a prompt indicating that the subject should wait another hour before receiving the dose. The subject, ignoring the prompt, proceeds with actuating the device. After the delivery of the ketamine, the smartphone generates a report that although the correct subject received the ketamine, the dose was administered at a time inconsistent with the prescription (e.g., the subject received the dose too early).

### Example 15: Subject Change after Identification

To begin, the process of Example 1 is performed. The subject in need of ketamine, rather than actuating the device, hands the device to a friend who wants to receive the ketamine. The smartphone, which no longer recognizes the subject because they have moved out of view, provides an indicator to the device, which prevents the device from actuating.

### Example 16: Subject Change after Identification

To begin, the process of Example 1 is performed. The subject in need of ketamine, rather than actuating the device, hands the device to a friend who wants to receive the ketamine. The smartphone, which no longer recognizes the subject because they have moved out of view, displays a prompt indicating that the smartphone does not recognize the subject and that the device should not be actuated. Instead, the friend actuates the device. Later, upon recognizing the device was actuated by recognizing the un-concealed marker without the housing, the smartphone generates a report that the device was actuated with an unidentified patient.

### Example 17: Guidance and Training

A nasal fluid delivery device is adapted by printing two fiducial marker ArUco code stickers to surfaces of the device during manufacturing, as shown in FIG. 7A. These markers and system provide simultaneous detection and tracking of device, relative subject face datums and position. This enables the system to provide device positional guidance and feedback to the subject (i.e. to desired specifications). The multiple ArUco codes also allow for precision device position tracking and enables detection of device insertion.

### Example 18: Full/Fractional Dose

A nasal fluid delivery device is adapted by printing two fiducial marker ArUco code stickers to surfaces of the device during manufacturing, as shown in FIG. 8A. The stickers are printed at a position such that they are visible during use of the device and are moved relative to each other during actuation of the device. A system is programmed to recognize the difference in movement and detect full or fractional dose. This is reported and can be used to manage medication adherence and help alert, train, and correct subject's behaviour for future doses.

### Example 19: Delivery Speed

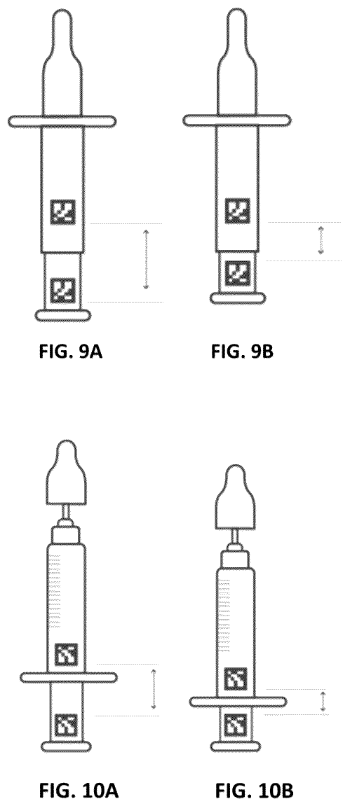

A nasal fluid delivery device is adapted by printing two fiducial marker ArUco code stickers to surfaces of the device during manufacturing, as shown in FIG. 9A. The stickers are printed at a position such that they are visible during use of the device and are moved relative to each other during actuation of the device. A system is programmed to detect speed of delivery. This is reported and can be used to manage medication adherence and help alert, train and correct subject's behaviour for future doses.

### Example 20: Adaption of an Inhaler Device to Allow for Medicament Verification

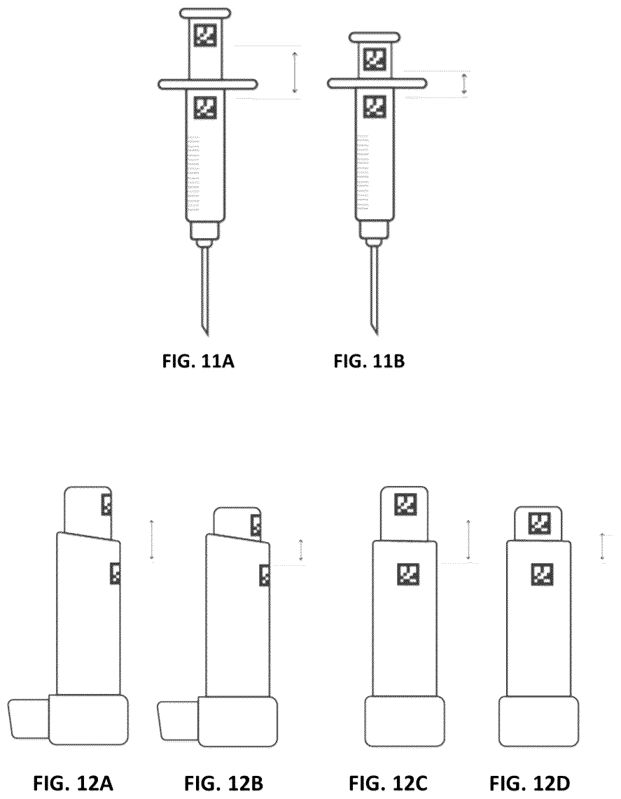

An inhaler device is adapted by the application of two fiducial marker ArUco code stickers to the device, as shown in FIG. 12A. The stickers are positioned such that they are visible during use of the device and are moved relative to each other during actuation of the device. A system is programed to recognize the adapted device in an unactuated configuration and in an actuated configuration.

### Example 21: Adaption of a Nasal Fluid Delivery Device to Allow for Medicament Verification

A nasal fluid delivery device is adapted by printing two fiducial marker ArUco code stickers to surfaces of the device during manufacturing, as shown in FIG. 14A. The stickers are printed at a position such that they are visible during use of the device and are moved relative to each other during actuation of the device. A system is programed to recognize the adapted device in an unactuated configuration and in an actuated configuration.

### Example 21: Adaption of a Device to Allow for Multi-Dose Medicament Verification

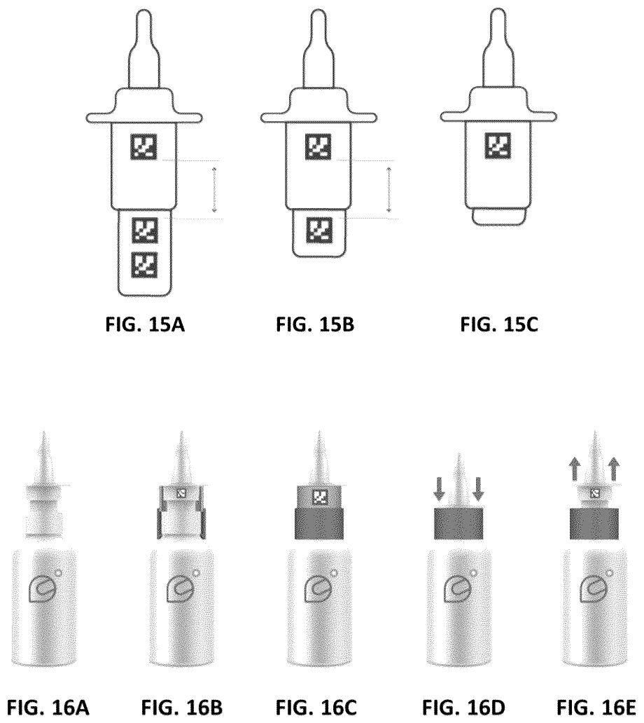

A multi-dose intranasal delivery device is adapted by the application of three fiducial marker ArUco codes to the device, as shown in FIG. 15A. The markers are positioned such that they are visible during use of the device and two markers are moved relative to a stationary marker during actuation of the device. A system is programed to recognize the adapted device in an unactuated configuration, in a partially actuated configuration corresponding to delivery of a single dose, and in a fully-actuated configuration corresponding to delivery of a second dose.

### Example 22: Adaption of a Device to Allow for Multi-Dose Medicament Verification

A delivery device is adapted by the application of two fiducial marker ArUco codes and inner and outer rings attached to the device, as shown in FIG. 16A-E. In an unactuated configuration a first ArUco code is visible on the inner sleeve. When the device is actuated, the inner sleeve is hidden behind the outer sleeve. Post actuation, the inner sleeve remains hidden behind the outer sleeve and the second ArUco code becomes visible. Actuation detection logic is based on detection that the ArUco code on the inner blue sleeve is no longer visible and is further confirmed by detecting the appearance of the second ArUco code marking.

### Example 23: Adaption of a Device to Allow for Multi-Dose Medicament Verification

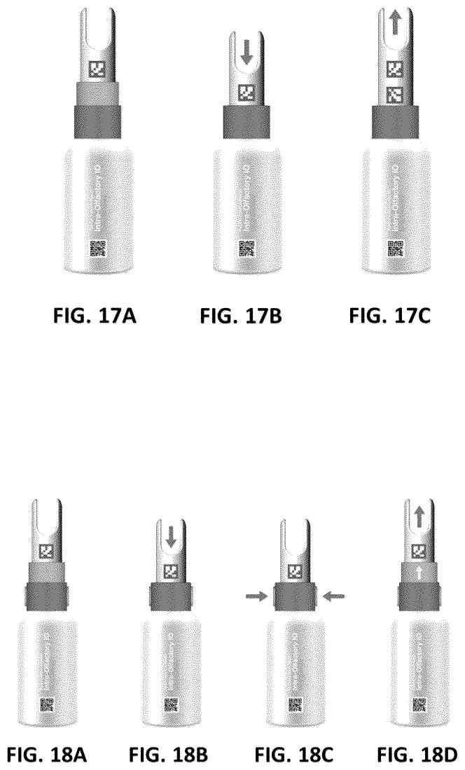

A dual-inserter intranasal delivery device includes two fiducial marker ArUco codes and inner and outer rings attached to the device, as shown in FIG. 17A-C. In an unactuated configuration a first ArUco code is visible above the inner sleeve. In an actuated configuration the inner sleeve is hidden behind the outer sleeve and the first ArUco code remains visible above the outer sleeve. Post actuation the inner sleeve remains hidden behind the outer sleeve and the two ArUco codes are visible above the outer sleeve. Actuation detection logic is based on first detecting the disappearance of the blue inner sleeve and as further confirmed when both ArUco codes are detected.

### Example 24: Adaption of a Device to Allow for Multi-Dose Medicament Verification

A dual-inserter intranasal delivery device includes a fiducial marker ArUco code and inner and outer rings attached to the device, as shown in FIG. 18A-D. In an unactuated configuration the ArUco code is visible above the inner sleeve. In an actuated configuration, the inner sleeve is hidden behind the outer sleeve and the ArUco code remains visible above the outer sleeve. Post actuation push buttons on either side of the cap are actuated to return the device to its original state, with the ArUco code visible above the inner sleeve. Actuation detection logic is based on detection of the disappearance of the inner blue ring and detection of the ArUco code and the inner blue ring after actuation.

### Example 25: Improper Position of a Non-Smart Device

A subject in need of ketamine opens the application on their smartphone that provides guidance to use the device. The subject turns on the camera and follows the prompts on the application. They place their face and the device as instructed in view of the camera on the smartphone. The smartphone captures one or more images of the subject's face and verifies the identity of the subject. However, the device moves away from the nose of the subject and into an improper position. The smartphone is monitoring the entire event and verifies the device is now in an improper position for dispersion of ketamine. The smartphone displays a message to the subject not to actuate the device and provides a recommendation to correct the position. The subject follows the recommendation to correct the position. The smartphone verifies correct positioning of the device and displays a prompt for the subject to actuate the device.

### Example 26: Improper Use of a Non-Smart Device

A subject has been prescribed ketamine but the next dose is not due. The subject nevertheless opens the application on their smartphone that provides guidance to use the device. The subject turns on the camera and follows the prompts on the application. They place their face and the device as instructed in view of the camera on the smartphone. The smartphone captures one or more images of the subject's face and verifies the identity of the subject. The smartphone registers that the subject should not be administering ketamine at the time. The smartphone displays a warning and a recommendation to wait to use the device. The subject ignores the warning and proceeds to actuate the device. The smartphone captures the actuation event on the camera and sends a notification of the improper use to the health care provider.

### Enumerated Embodiments

Enumerated embodiment 1. A method for delivery of a composition to a target region of a subject, comprising:
a. capturing one or more images of the subject;
b. identifying the subject based on the one or more images of the subject and one or more criteria, wherein the one or more criteria comprise the subject being in need of the composition;
c. capturing one or more images of a device, wherein the device comprises a marker and a housing, wherein the device is configured to transition between a plurality of configuration states based on a position of the marker and the housing;
d. identifying the device as being in a first configuration state of the plurality of configuration states based on the one or more images of the device, wherein the marker is not concealed by the housing in the first configuration state;
e. at least one of:
   i. providing an indicator to deliver the composition to the target region based on identifying the device as being in the first configuration state and identifying the subject based on the one or more criteria; or
   ii. displaying a recommendation to deliver the composition to the target region based on identifying the device as being in the proper configuration state and identifying the subject based on the one or more criteria; or
   iii. verifying actuation of the device based on a change in the configuration state of the device, thereby verifying delivery of the composition of the subject.
Enumerated embodiment 2. The method of any one of the enumerated embodiments, wherein the marker is a fiducial marker.
Enumerated embodiment 3. The method of any one of the enumerated embodiments wherein the first configuration state of the plurality of configuration states indicates an alignment of the device with respect to the target region of the subject, and/or with respect to an alignment region of the subject.
Enumerated embodiment 4. The method of any one of the enumerated embodiments, wherein the target region and/or the alignment region is the nasal cavity of the subject, an olfactory region of the subject, an orifice, a mouth, or the columella of the subject.
Enumerated embodiment 5. The method of any one of the enumerated embodiments, further comprising identifying the device as being in a second configuration state of the plurality of configuration states, wherein the marker is at least partially concealed by the housing and the housing is visible.
Enumerated embodiment 6. The method of any one of the enumerated embodiments, wherein identifying the device being in the second configuration state occurs after identifying the device being in the first configuration state.
Enumerated embodiment 7. The method of any one of the enumerated embodiments, further comprising identifying the device as being in a third configuration state of the plurality of configuration states, wherein the marker is at least partially concealed by the housing and the housing is not visible.
Enumerated embodiment 8. The method of any one of the enumerated embodiments, wherein identifying the device being in the third configuration state occurs after identifying the device being in the second configuration state.
Enumerated embodiment 9. The method of any one of the enumerated embodiments, further comprising identifying the device as being in a fourth configuration state of the plurality of configuration states, wherein the marker is not concealed by the housing and the housing is not visible.
Enumerated embodiment 10. The method of any one of the enumerated embodiments, wherein identifying the device being in the fourth configuration state occurs after identifying the device being in the third configuration state.
Enumerated embodiment 11. The method of any one of the enumerated embodiments, wherein the fourth configuration state confirms actuation of the device.
Enumerated embodiment 12. The method of any one of the enumerated embodiments, wherein the fourth configuration state indicates the device does not comprise the composition.
Enumerated embodiment 13. The method of any of the enumerated embodiments, wherein the device further comprises a second housing transitionable from the second configuration state to the third configuration state.
Enumerated embodiment 14. The method of any of the enumerated embodiments, wherein the second housing is not visible in the first configuration state and the second configuration state, wherein the second housing is visible in the third configuration state and the fourth configuration state.
Enumerated embodiment 15. The method of any one of the enumerated embodiments, wherein the marker is a QR code.
Enumerated embodiment 16. The method of any one of the enumerated embodiments, wherein the marker is an ArUco marker.
Enumerated embodiment 17. The method of any one of the enumerated embodiments, wherein the device being in the first configuration state indicates the device can be actuated.
Enumerated embodiment 18. The method of any one of the enumerated embodiments, further comprising monitoring the device and/or the subject.
Enumerated embodiment 19. The method of any one of the enumerated embodiments, wherein, based on the monitoring, the method comprises providing a second indicator, wherein the second indicator prevents the device from delivering the composition.
Enumerated embodiment 20. The method of any one of the enumerated embodiments, wherein the second indicator is provided based on the subject being unidentifiable during the monitoring or the device not being in the first configuration state during the monitoring.
Enumerated embodiment 21. The method of any one of the enumerated embodiments, wherein, based on the monitoring, the method comprises providing a third indicator after providing the second indicator, wherein the third indicator allows the device to deliver the composition.
Enumerated embodiment 22. The method of any one of the enumerated embodiments, wherein the third indicator is provided based on the subject becoming identifiable during the monitoring or the device being in the first configuration state during the monitoring after previously being in a different configuration state.
Enumerated embodiment 23. The method of any one of the enumerated embodiments, wherein the marker comprises one or more submarkers.
Enumerated embodiment 24. The method of any one of the enumerated embodiments, wherein, in the second configuration state, a first submarker of the one or more submarkers is concealed by the housing and a second submarker of the one or more submarkers is not concealed by the housing and the housing is not concealed.
Enumerated embodiment 25. The method of any one of the enumerated embodiments, wherein, in the first configuration state, the first submarker of the one or more submarkers is not concealed, and the second submarker of the one or more submarkers is not concealed, and the housing is not concealed.
Enumerated embodiment 26. The method of any one of the enumerated embodiments, wherein, in the third configuration state, the first submarker of the one or more submarkers is concealed, a second submarker of the one or more submarkers is not concealed, and the housing is concealed.
Enumerated embodiment 27. The method of any one of the enumerated embodiments, wherein the device is partially inserted into a nasal cavity of the subject, causing the device to transition from the first configuration state to the second configuration state.
Enumerated embodiment 28. The method of any one of the enumerated embodiments, wherein the device is partially inserted into the nasal cavity of the subject at a prescribed depth and angle.
Enumerated embodiment 29. The method of any one of the enumerated embodiments, further comprising determining an alignment state of the device and the subject based upon a position of the marker.
Enumerated embodiment 30. The method of any one of the enumerated embodiments, further comprising determining an alignment state of the device and the subject based upon a position of the marker relative to one or more subject facial features, the one or more criteria, or combinations thereof.
Enumerated embodiment 31. The method of any one of the enumerated embodiments, wherein the position comprises the alignment state of the device, and wherein the method further comprises providing a recommendation to adjust the alignment state of the device if it is determined the alignment state is improper relative to the one or more subject facial features.
Enumerated embodiment 32. The method of any one of the enumerated embodiments, wherein the identifying the subject comprises analyzing an image amongst the one or more images, wherein the image is of the subject's face, and associating at least 68 data points to facial features of the subject.
Enumerated embodiment 33. The method of any one of the enumerated embodiments, wherein the data points associated with facial features of the subject comprise at least two points corresponding to a nose bridge and a tip of a nose of the subject.
Enumerated embodiment 34. The method of any one of the enumerated embodiments, wherein the data points associated with facial features of the subject are used to produce a 3D facial map.
Enumerated embodiment 35. The method of any one of the enumerated embodiments, wherein the 3D facial map is produced using a machine learning algorithm.
Enumerated embodiment 36. The method of any one of the enumerated embodiments, wherein the machine learning algorithm calculates the position of a facial feature based on an image not present in the captured one or more images.
Enumerated embodiment 37. The method of any one of the enumerated embodiments, wherein the marker comprises at least two submarkers which indicate a configuration state of the device.
Enumerated embodiment 38. The method of any one of the enumerated embodiments, further comprising determining a configuration state of the device based upon the position of the marker.
Enumerated embodiment 39. The method of any one of the enumerated embodiments, further comprising determining an alignment state of the device based upon the position of the marker relative to one or more subject facial features, the one or more subject indicia, or combinations thereof.
Enumerated embodiment 40. The method of any one of the enumerated embodiments, wherein the 3D facial map comprises facial landmarks not directly measured by the camera, wherein the facial landmarks not directly measured by the camera are extrapolated using the machine learning algorithm and input based on the one or more data points.
Enumerated embodiment 41. The method of any one of the enumerated embodiments, further comprising providing a side profile of the subject to the machine learning algorithm.
   Enumerated embodiment 42. The method of any one of the enumerated embodiments, wherein the side profile is used to calculate the nose angle of the subject.
Enumerated embodiment 43. The method of any one of the enumerated embodiments, wherein the nose angle is calculated using a formula

$${tan}^{- 1} ( \frac{\vert {z}_{2} - {z}_{1} \vert}{\vert {y}_{2} - {y}_{1} \vert} ) * \frac{180}{π} ,$$

by comparing a depth at a middle of the nose z1 and a depth at top of nose z2, where (x1,y1,z1) and (x2,y2,z2) are the Cartesian-coordinates of the two points, respectively.
Enumerated embodiment 44. The method of any one of the enumerated embodiments, further comprising measuring the nose angle of the subject by comparing the tip of the nose of the subject with a depth of the nose of the subject at a top of the nose bridge of the nose.
Enumerated embodiment 45. The method of any one of the enumerated embodiments, wherein measuring the nose angle of the subject is with an accuracy of +/−2 degrees, +/−3 degrees, +/−4 degrees, +/−5 degrees, +/−6 degrees, +/−7 degrees, +/−8 degrees, +/−9 degrees, +/−10 degrees, +/−11 degrees, +/−12 degrees, +/−13 degrees, +/−14 degrees, or +/−15 degrees.
Enumerated embodiment 46. The method of any one of the enumerated embodiments, further comprising comparing a position of the device in the nasal cavity of the subject relative to a database of video or images files of a device in a nasal cavity of the subject, and indicating a proper or improper alignment state of the device based upon similarity in one or more features between a captured image or video file of the subject, and the database of video or image files.
Enumerated embodiment 47. The method of any one of the enumerated embodiments, wherein the position of the device is determined by applying a bounding box around the marker.
Enumerated embodiment 48. The method of any one of the enumerated embodiments, wherein the position is determined based upon a pitch or a yaw of the device.
Enumerated embodiment 499. The method of any one of the enumerated embodiments, wherein the pitch or the yaw of the device is calculated based upon the shape of the bounding box.
Enumerated embodiment 50. The method of any one of the enumerated embodiments, further comprising using a position of the housing compared to the position of the first fiducial marker to calculate a pitch of the device.
Enumerated embodiment 51. The method of any one of the enumerated embodiments, wherein the pitch or the yaw of the device is calculated based upon a measurement of the marker to a projection of a plane defined by the measurement of the marker.
Enumerated embodiment 52. The method of any one of the enumerated embodiments, wherein the projection of the plane defined by the known measurement of the marker is captured as a 3D object projected into a 2D space.
Enumerated embodiment 53. The method of any one of the enumerated embodiments, wherein the pitch of the device is calculated based on the projection onto the plane using a formula:

$${cos}^{- 1} ( \frac{observed height}{actual height} ) * \frac{180}{π} ,$$

wherein observed height is an observed height of the fiducial marker, and actual height is an actual height of the fiducial marker.
Enumerated embodiment 54. The method of any one of the enumerated embodiments, wherein the camera comprises a depth camera, wherein the pitch of the device is calculated based upon the projection onto the plane using a formula:

$${sin}^{- 1} ( \frac{\vert {z}_{2} - {z}_{1} \vert}{λ} ) * \frac{180}{π} ,$$

wherein λ is an actual height of the fiducial marker, wherein z₁ is a depth at a top of the marker and z₂ is a depth at the bottom of the marker.
Enumerated embodiment 55. The method of any one of the enumerated embodiments, wherein the pitch of the device is calculated with an accuracy of +/−2°, +/−3°, +/−4°, +/−5°, +/−6°, +/−7°, +/−8°, +/−9°, +/−10°, +/−11°, +/−12°, +/−13°, +/−14°, or +/−15° when the pitch is greater than 20°, or ±10°, +11°, +12°, +13°, +14°, or ±15° when the pitch is or is lower than 20°.
Enumerated embodiment 56. The method of any one of the enumerated embodiments, wherein the accuracy of the pitch relative to the nose of the subject is calculated based on an accuracy of the pitch of the device and an accuracy of the pitch of the nose.
Enumerated embodiment 57. The method of any one of the enumerated embodiments, wherein the yaw of the device is calculated based upon a vertical edge of the bounding box.
Enumerated embodiment 58. The method of any one of the enumerated embodiments, wherein the yaw of the device is calculated based upon a projection onto the plane using a formula:

$${tan}^{- 1} ( \frac{{x}_{1} - {x}_{2}}{{y}_{1} - {y}_{2}} ) * \frac{180}{π} ,$$

wherein x1 is a horizontal coordinate at a top corner of the marker, x2 is a horizontal coordinate at a bottom corner of the marker, y₁ is a vertical coordinate at a top corner of the marker, and y2 is a vertical coordinate at a bottom corner of the marker. Enumerated embodiment 59. The method of any one of the enumerated embodiments, wherein the yaw is calculated with an accuracy of ±3°, ±4°, ±5°, ±6°, ±7°, ±8°, ±9°, ±10°, ±11°, ±12°, ±13°, ±14°, or ±15°
Enumerated embodiment 60. The method of any one of the enumerated embodiments, wherein the accuracy of the yaw relative to the subject's nose is calculated based on an accuracy of a yaw of the device and an accuracy of a yaw of the nose.
Enumerated embodiment 61. The method of any one of the enumerated embodiments, wherein the recommendation comprises feedback to the subject as to the alignment state of the device, wherein the subject is directed to adjust alignment state of the device if it is determined the device is in an improper alignment state.
Enumerated embodiment 62. The method of any one of the enumerated embodiments, further comprising providing feedback to the subject as to the alignment state of the device, wherein the subject is directed to adjust the alignment state of the device if it is determined the device is in an improper alignment state.
Enumerated embodiment 63. The method of any one of the enumerated embodiments, further comprising providing feedback as to the alignment state of the device, wherein the subject is provided an indication of a proper alignment state of the device prior to actuating the device.
Enumerated embodiment 64. The method of any one of the enumerated embodiments, further comprising continuously tracking and continuously calculating the position of the one or more subject facial features relative to the camera.
Enumerated embodiment 65. The method of any one of the enumerated embodiments, wherein the identifying the subject comprises using a machine learning algorithm to determine a 3D facial surface map of the subject with the camera.
Enumerated embodiment 66. The method of any one of the enumerated embodiments, further comprising collecting data corresponding to facial features of the subject.
Enumerated embodiment 67. The method of any one of the enumerated embodiments, further comprising calculating an angle of the nose, an angle of the head, or position of a nasal cavity using at least a portion of the data points associated with facial features of the subject.
Enumerated embodiment 68. The method of any one of the enumerated embodiments, wherein the identifying the subject comprises using a machine learning algorithm to determine a 3D facial surface of the subject with the camera without using a depth sensor.
Enumerated embodiment 69. The method of any one of the enumerated embodiments, wherein the identifying the subject comprises comparing detected facial features of the subject to a known or verified image of the subject to identify the subject with at least about 70%, at least about 75%, at least about 80%, at least about 85%, at least about 90%, or at least about 95% accuracy.
Enumerated embodiment 70. The method of any one of the enumerated embodiments, further comprising tracking a position of the nose or face of the subject relative to the camera, wherein tracking the position of the nose or face of the subject comprises extrapolating an angle of the nose based upon the at least two points one or more points corresponding to a nose bridge and a tip of a nose of the subject.
Enumerated embodiment 71. The method of any one of the enumerated embodiments, wherein identifying the subject using one or more subject indicia comprises applying a facial recognition algorithm to identify the subject.
Enumerated embodiment 72. The method of any one of the enumerated embodiments, wherein the one or more subject indicia comprises one or more facial features of the subject.
Enumerated embodiment 73. The method of any one of the enumerated embodiments, wherein the identifying the subject comprises matching the one or more facial features of the subject to a data file comprising data corresponding to one or more facial features of the subject.
Enumerated embodiment 74. The method of any one of the enumerated embodiments, wherein the identifying the subject comprises matching the one or more facial features of the subject to a data file comprising a verified image of the subject.
Enumerated embodiment 75. The method of any one of the enumerated embodiments, wherein the machine learning algorithm isolates a face within an individual frame of a captured video from the camera for analysis.
Enumerated embodiment 76. The method of any one of the enumerated embodiments, wherein identifying the subject using one or more subject indicia comprises applying a facial recognition algorithm to identify the subject by matching the one or more subject indicia to a known facial scan of the subject.
Enumerated embodiment 77. The method of any one of the enumerated embodiments, wherein identifying the subject comprises instructing the subject to position a face of the subject within a target view field of the camera.
Enumerated embodiment 78. The method of any one of the enumerated embodiments, wherein identifying the subject comprises indicating to the subject when the face of the subject is correctly positioned within the target view field of the camera.
Enumerated embodiment 79. The method of any one of the enumerated embodiments, further comprising continuously identifying the subject during performance of the method of any one of the enumerated embodiments, or continuously identifying the subject at multiple time points during performance of the method of any one of the enumerated embodiments.
Enumerated embodiment 80. The method of any one of the enumerated embodiments, further comprising continuously identifying the subject during the actuating of the device, verifying actuation of the device, the determining the alignment state of the device, or any combination thereof.
Enumerated embodiment 81. The method of any one of the enumerated embodiments, further comprising re-identifying the subject if the subject leaves the field of view of the camera during performance of the method.
Enumerated embodiment 82. The method of any one of the enumerated embodiments, further comprising identifying the device by scanning and identifying indica located on the device with the computing device.
Enumerated embodiment 83. The method of any one of the enumerated embodiments, further comprising verifying the device corresponds to a prescription by referencing matching the device to a data file comprising data corresponding to a prescription.
Enumerated embodiment 84. The method of any one of the enumerated embodiments, further comprising verifying the device corresponds to a prescription belonging to the subject based upon a positive device identification, a positive prescription identification, a positive subject identification, or combinations thereof.
Enumerated embodiment 85. The method of any one of the enumerated embodiments, further comprising identifying an impediment to identifying the subject or verifying the device, and prompting the subject to correct the impediment.
Enumerated embodiment 86. The method of any one of the enumerated embodiments, wherein the impediment comprises the subject or the device being positioned too far away from the camera, an improper lighting state, a physical obstruction in view of the camera, an improper subject head angle, a subject position out of view of the camera, or any combination thereof.
Enumerated embodiment 87. The method of any one of the enumerated embodiments, further comprising continuously identifying a position of the device during performance of the claimed method, or continuously identifying a position of the device at multiple time points during performance of the claimed method.
Enumerated embodiment 88. The method of any one of the enumerated embodiments, further comprising continuously identifying a position of the device during the actuating of the device, verifying actuation of the device, the determining the alignment state of the device, or any combination thereof.
Enumerated embodiment 89. The method of any one of the enumerated embodiments, further comprising verifying a configuration state of the device based upon whether the housing is visible.
Enumerated embodiment 90. The method of any one of the enumerated embodiments, further comprising prompting the subject to actuate the device.
Enumerated embodiment 91. The method of any one of the enumerated embodiments, further comprising wherein the first indicator prompts the subject to actuate the device.
Enumerated embodiment 92. The method of any one of the enumerated embodiments, wherein the third indicator prompts the subject to re-actuate the device.
Enumerated embodiment 93. The method of any one of the enumerated embodiments, wherein the device comprises a securing mechanism preventing actuation of the device.
Enumerated embodiment 94. The method of any one of the enumerated embodiments, wherein the device is in communication with the computing device.
Enumerated embodiment 95. The method of any the enumerated embodiments, wherein the securing mechanism is released upon verifying a positive subject identification, a positive prescription identification, a proper alignment state of the device, or combinations thereof.
Enumerated embodiment 96. The method of any one of the enumerated embodiments, wherein the securing mechanism is released upon a signal transmitted from the computing device to the device.
Enumerated embodiment 97. The method of any one of the enumerated embodiments, wherein the recommendation comprises one or more prompts.
Enumerated embodiment 98. The method of any one of the enumerated embodiments, wherein verifying delivery of the composition to the subject comprises maintaining a continuous positive subject identification throughout the actuating the device.
Enumerated embodiment 99. The method of any one of the enumerated embodiments, further comprising identifying a negative subject identification and a positive prescription identification, and not releasing the securing mechanism, preventing actuation of the device.
Enumerated embodiment 100. The method of any one of the enumerated embodiments, further comprising identifying a negative subject identification and a positive prescription identification, and generating a report.
Enumerated embodiment 101. The method of any one of the enumerated embodiments, wherein the data file comprising data corresponding to one or more facial features of the subject, the data file comprising a verified image of the subject, the data file comprising data corresponding to a prescription are stored locally on the computing device.
Enumerated embodiment 102. The method of any one of the enumerated embodiments, wherein the data file comprising data corresponding to one or more facial features of the subject, the data file comprising a verified image of the subject, the data file comprising data corresponding to a prescription are stored remotely from the computing device.
Enumerated embodiment 103. The method of any one of the enumerated embodiments, further comprising capturing one or more images of the subject when the device is actuated.
Enumerated embodiment 104. The method of any one of the enumerated embodiments, further comprising capturing one or more images of the subject during the identifying the subject.
Enumerated embodiment 105. The method of any one of the enumerated embodiments, wherein the marker comprises a bar code, a QR code, or other camera readable code comprising drug data, prescription data, or device identification data.
Enumerated embodiment 106. The method of any one of the enumerated embodiments, wherein the marker comprises an ArUco marker.
Enumerated embodiment 107. The method of any one of the enumerated embodiments, further comprising updating the machine learning algorithm based upon a captured image or video file of the subject.
Enumerated embodiment 108. The method of any one of the enumerated embodiments, further comprising detecting an object or obstruction within a reference frame.
Enumerated embodiment 109. The method of any one of the enumerated embodiments, further comprising identifying objects or obstructions within the reference frame based upon a shape, a contour, a color, or a texture of the object or obstruction.
Enumerated embodiment 110. The method of any one of the enumerated embodiments, further comprising localizing a position of the object or obstruction within a reference frame.
Enumerated embodiment 111. The method of any one of the enumerated embodiments, further comprising measuring a distance between the camera and the subject or device using a relative scale, wherein the relative scale increases the accuracy of the measurement.
Enumerated embodiment 112. The method of any one of the enumerated embodiments, further comprising detecting the timing of delivery of the composition.
Enumerated embodiment 113. The method of any one of the enumerated embodiments, wherein detecting the timing of delivery of the composition comprises detecting a speed of actuation.
Enumerated embodiment 114. The method of any one of the enumerated embodiments, wherein patient identification, detection of device positioning, detection of device actuation, and/or corrective guidance are performed simultaneously.
Enumerated embodiment 115. The method of any one of the enumerated embodiments, further comprising generating a data report comprising subject identity, device serial number, drug identity, date, time, location, a video of performance of the claimed method, a photo of performance of the claimed method, an actuation event, face position at actuation, device position at actuation, an actuation confirmation, incorrect dose alert, tampering alert or any combination thereof.
Enumerated embodiment 116. The method of any one of the enumerated embodiments, further comprising storing data in a database following performance of any step of the method.
Enumerated embodiment 117. The method of any one of the enumerated embodiments, wherein the data comprises image or video data.
Enumerated embodiment 118. The method of any one of the enumerated embodiments, wherein the image or video data is stored as a JSON object structure.
Enumerated embodiment 119. The method of any one of the enumerated embodiments, wherein the JSON object structure comprises keys mapping the positioning state of the device, the identifying the subject, the actuating the device, or any combination thereof relative to a state of performance of the method.
Enumerated embodiment 120. The method of any one of the enumerated embodiments, wherein the composition comprises a medicament, vaccine, biologic, small molecule, cell line, or therapeutic.
Enumerated embodiment 121. A system, comprising:
one or more processors; and
a memory comprising executable instructions which, when executed by the one or more processors, cause the system to:
   capture one or more images of a subject;
   identify the subject based on the one or more images of the subject and one or more criteria, wherein the one or more criteria comprises the subject being in need of the composition;
   capture one or more images of a device, wherein the device comprises a marker and a housing, wherein the device is configured to transition between a plurality of configuration states based on a position of the marker and the housing;
   identify the device as being in a first configuration state of the plurality of configuration states based on the one or more images of the device, wherein the marker is not concealed by the housing in the first configuration state;
   at least one of:
      provide an indicator to deliver the composition to the target region based on identifying the device as being in the first configuration state and identifying the subject based on the one or more criteria; or
      display a recommendation to deliver the composition to the target region based on identifying the device as being in the proper configuration state and identifying the subject based on the one or more criteria; or verify actuation of the device based on a change in the configuration state of the device, thereby verifying delivery of the composition of the subject.
Enumerated embodiment 122. The system of any one of the enumerated embodiments, wherein the marker is a fiducial marker.
Enumerated embodiment 123. The system of any one of the enumerated embodiments, wherein the first configuration state of the plurality of configuration states indicates an alignment of the device with respect to the target region of the subject, and/or with respect to an alignment region of the subject.
Enumerated embodiment 124. The system of any one of the enumerated embodiments, wherein the target region and/or the alignment region is an orifice of the subject, the mouth of the subject, the nasal cavity of the subject, an olfactory region of the subject, or the columella of the subject.
Enumerated embodiment 125. The system of any one of the enumerated embodiments, wherein the marker is at least partially concealed by the housing and the housing is visible in a second configuration state of the plurality of configuration states.
Enumerated embodiment 126. The system of any one of the enumerated embodiments, wherein the marker is at least partially concealed by the housing and the housing is not visible in a third configuration state of the plurality of configuration states.
Enumerated embodiment 127. The system of any one of the enumerated embodiments, wherein the marker is not concealed by the housing and the housing is not visible in a fourth configuration state of the plurality of configuration states.
Enumerated embodiment 128. The system of any one of the enumerated embodiments, wherein the device further comprises a second housing transitionable from the second configuration state to the third configuration state.
Enumerated embodiment 129. The system of any one of the enumerated embodiments, wherein the second housing is not visible in the first configuration state and the second configuration state, wherein the second housing is visible in the third configuration state and the fourth configuration state.
Enumerated embodiment 130. The system of any one of the enumerated embodiments, wherein the marker is a QR code.
Enumerated embodiment 131. The system of any one of the enumerated embodiments, wherein the marker is an ArUco marker.
Enumerated embodiment 132. The system of any one of the enumerated embodiments, wherein the device being in the first configuration state indicates the device can be actuated.
Enumerated embodiment 133. The system of any one of the enumerated embodiments, wherein the one or more processors are further configured to cause the system to monitor the device and/or the subject.
Enumerated embodiment 134. The system of any one of the enumerated embodiments, wherein, the one or more processors are further configured to provide a second indicator, wherein the second indicator prevents the device from delivering the composition.
Enumerated embodiment 135. The system of any one of the enumerated embodiments, wherein the second indicator is provided based on the subject not being identifiable during monitoring or the device not being in the first configuration state during monitoring.
Enumerated embodiment 136. The system of any one of the enumerated embodiments, wherein, the one or more processors are further configured to provide a third indicator after providing the second indicator, wherein the third indicator allows the device to deliver the composition.
Enumerated embodiment 137. The system of any one of the enumerated embodiments, wherein the third indicator is provided based on the subject becoming identifiable during monitoring or the device being in the first configuration state during monitoring after previously being in a different configuration state.
Enumerated embodiment 138. The system of any one of the enumerated embodiments, wherein the marker has one or more submarkers.
Enumerated embodiment 139. The system of any one of the enumerated embodiments, wherein, in the second configuration state, a first submarker of the one or more submarkers is concealed by the housing and a second submarker of the one or more submarkers is not concealed by the housing and the housing is not concealed.
Enumerated embodiment 140. The system of any one of the enumerated embodiments, wherein, in the first configuration state, the first submarker of the one or more submarkers is not concealed, and the second submarker of the one or more submarkers is not concealed, and the housing is not concealed.
Enumerated embodiment 141. The system of any one of the enumerated embodiments, wherein, in the third configuration state, the first submarker of the one or more submarkers is not concealed, a second submarker of the one or more submarkers is concealed, and the housing is concealed.
Enumerated embodiment 142. The system of any one of the enumerated embodiments, wherein the device is partially inserted into a nasal cavity of the subject, causing the device to transition from the first configuration state to the second configuration state.
Enumerated embodiment 143. The system of any one of the enumerated embodiments, wherein the device is partially inserted into the nasal cavity of the subject at a prescribed depth and angle.
Enumerated embodiment 144. The system of any one of the enumerated embodiments, wherein the one or more processors are further configured to determine an alignment state of the device and the subject based upon a position of the marker.
Enumerated embodiment 145. The system of any one of the enumerated embodiments, wherein the one or more processors are further configured to determine an alignment state of the device and the subject based upon a position of the marker relative to one or more subject facial features, the one or more criteria, or combinations thereof.
Enumerated embodiment 146. The system of any one of the enumerated embodiments, wherein the position comprises the alignment state of the device, and wherein the one or more processors are further configured to provide a recommendation to adjust the alignment state of the device if it is determined the alignment state is improper relative to the one or more subject facial features.
Enumerated embodiment 147. The system of any one of the enumerated embodiments, wherein the one or more processors being configured to identify the subject comprises the one or more processors being configured to analyze an image amongst the one or more images, wherein the image is of the subject's face, and associating at least 68 data points to facial features of the subject.
Enumerated embodiment 148. The system of any one of the enumerated embodiments, wherein the data points associated with facial features of the subject comprise at least two points corresponding to a nose bridge and a tip of a nose of the subject.
Enumerated embodiment 149. The system of any one of the enumerated embodiments, wherein the data points associated with facial features of the subject are used to produce a 3D facial map.
Enumerated embodiment 150. The system of any one of the enumerated embodiments, wherein the 3D facial map is produced using a machine learning algorithm.
Enumerated embodiment 151. The system of any one of the enumerated embodiments, wherein the machine learning algorithm calculates the position of a facial feature based on an image not present in the captured one or more images.
Enumerated embodiment 152. The system of any one of the enumerated embodiments, wherein the marker comprises at least two submarkers which indicate a configuration state of the device.
Enumerated embodiment 153. The system of any one of the enumerated embodiments, wherein the one or more processors are further configured to determine a configuration state of the device based upon the position of the marker.
Enumerated embodiment 154. The system of any one of the enumerated embodiments, wherein the one or more processors are further configured to determine an alignment state of the device based upon the position of the marker relative to one or more subject facial features, the one or more subject indicia, or combinations thereof.
Enumerated embodiment 155. The system of any one of the enumerated embodiments, wherein the 3D facial map comprises facial landmarks not directly measured by the camera, wherein the facial landmarks not directly measured by the camera are extrapolated using the machine learning algorithm and input based on the one or more data points.
Enumerated embodiment 156. The system of any one of the enumerated embodiments, wherein the one or more processors are further configured to provide a side profile of the subject to the machine learning algorithm.
Enumerated embodiment 157. The system of any one of the enumerated embodiments, wherein the side profile is used to calculate the nose angle of the subject.
Enumerated embodiment 158. The system of any one of the enumerated embodiments, wherein the nose angle is calculated using a formula

$${tan}^{- 1} ( \frac{\vert {z}_{2} - {z}_{1} \vert}{\vert {y}_{2} - {y}_{12} \vert} ) * \frac{180}{π} ,$$

by comparing a depth at a middle of the nose (z1/y1/x1) and a depth at top of the nose (z2/y2/x2).
Enumerated embodiment 159. The system of any one of the enumerated embodiments, wherein the one or more processors are further configured to measure the nose angle of the subject by comparing the tip of the nose of the subject with a depth of the nose of the subject at a top of the nose bridge of the nose.
Enumerated embodiment 160. The system of any one of the enumerated embodiments, wherein a measurement of the nose angle of the subject is with an accuracy of +/−2 degrees, +/−3 degrees, +/−4 degrees, +/−5 degrees, +/−6 degrees, +/−7 degrees, +/−8 degrees, +/−9 degrees, +/−10 degrees, +/−11 degrees, +/−12 degrees, +/−13 degrees, +/−14 degrees or +/−15 degrees.
Enumerated embodiment 161. The system of any one of the enumerated embodiments, wherein the one or more processors are further configured to compare a position of the device in the nasal cavity of the subject relative to a database of video or images files of a device in a nasal cavity of the subject, and indicating a proper or improper alignment state of the device based upon similarity in one or more features between a captured image or video file of the subject, and the database of video or image files.
Enumerated embodiment 162. The system of any one of the enumerated embodiments, wherein the one or more processors are configured to apply a bounding box around the marker to determine the position of the device.
Enumerated embodiment 163. The system of any one of the enumerated embodiments, wherein the position is determined based upon a pitch or a yaw of the device.
Enumerated embodiment 164. The system of any one of the enumerated embodiments, wherein the pitch or the yaw of the device is calculated based upon the shape of the bounding box.
Enumerated embodiment 165. The system of any one of the enumerated embodiments, wherein the one or more processors are further configured to use a position of the housing compared to the position of the first fiducial marker to calculate a pitch of the device.
Enumerated embodiment 166. The system of any one of the enumerated embodiments, wherein the pitch or the yaw of the device is calculated based upon a measurement of the marker to a projection of a plane defined by the measurement of the marker.
Enumerated embodiment 167. The system of any one of the enumerated embodiments, wherein the projection of the plane defined by the known measurement of the marker is captured as a 3D object projected into a 2D space.
Enumerated embodiment 168. The system of any one of the enumerated embodiments, wherein the pitch of the device is calculated based upon the projection of the plane using a formula:

$${cos}^{- 1} ( \frac{observed height}{actual height} ) * \frac{180}{π} ,$$

wherein observed height is an observed height of the fiducial marker, and actual height is an actual height of the fiducial marker.
Enumerated embodiment 169. The system of any one of the enumerated embodiments, wherein the camera comprises a depth camera, wherein the pitch of the device is calculated based upon the projection of the plane using a formula:

$${sin}^{- 1} ( \frac{\vert {z}_{2} - {z}_{1} \vert}{λ} ) * \frac{180}{π} ,$$

wherein λ is an actual height of the fiducial marker, wherein z₁ is a depth at a top of the marker and z2 is a depth at a bottom of the marker.
Enumerated embodiment 170. The system of any one of the enumerated embodiments, wherein the pitch of the device is calculated with an accuracy of +/−2°, +/−3°, +/−4°, +/−5°, +/−6°, +/−7°, +/−8°, +/−9°, +/−10°, +/−11°, +/−12°, +/−13°, +/−14° or +/−15° when the pitch is greater than 20°, or ±10°, +/−11°, +/−12°, +/−13°, +/−14° or +/−15° when the pitch is or is lower than 20°.
Enumerated embodiment 171. The system of any one of the enumerated embodiments, wherein the accuracy of the pitch relative to the nose of the subject is calculated based on an accuracy of the pitch of the device and an accuracy of the pitch of the nose.
Enumerated embodiment 172. The system of any one of the enumerated embodiments, wherein the yaw of the device is calculated based upon a vertical edge of the bounding box
Enumerated embodiment 173. The system of any one of the enumerated embodiments, wherein the yaw of the device is calculated based upon a projection onto the plane using a formula:

$${tan}^{- 1} ( \frac{{x}_{1} - {x}_{2}}{{y}_{1} - {y}_{2}} ) * \frac{180}{π} ,$$

wherein x₁ is a horizontal coordinate at a top corner of the marker, x₂ is a horizontal coordinate at a bottom corner of the marker, y₁ is a vertical coordinate at the top corner of the marker, and y₂ is a vertical coordinate at the bottom corner of the marker.
Enumerated embodiment 174. The system of any one of the enumerated embodiments, wherein the yaw is calculated with an accuracy of ±3°, +/−4°, +/−5°, +/−6°, +/−7°, +/−8°, +/−9°, +/−10°, +/−11°, +/−12°, +/−13°, +/−14°, or +/−15°.
Enumerated embodiment 175. The system of any one of the enumerated embodiments, wherein the accuracy of the yaw relative to the subject's nose is calculated based on an accuracy of a yaw of the device and an accuracy of a yaw of the nose.
Enumerated embodiment 176. The system of any one of the enumerated embodiments, wherein the recommendation comprises feedback as to the alignment state of the device, wherein the feedback indicates the alignment state of the device should be adjusted if it is determined the device is in an improper alignment state.
Enumerated embodiment 177. The system of any one of the enumerated embodiments, wherein the one or more processors are further configured to provide feedback to the subject as to the alignment state of the device, wherein the subject is directed to adjust the alignment state of the device if it is determined the device is in an improper alignment state.
Enumerated embodiment 178. The system of any one of the enumerated embodiments, wherein the one or more processors are further configured to provide feedback as to the alignment state of the device, wherein an indication of a proper alignment state of the device is provided prior to actuating the device.
Enumerated embodiment 179. The system of any one of the enumerated embodiments, wherein the one or more processors are further configured to continuously track and continuously calculate the position of the one or more subject facial features relative to the camera.
Enumerated embodiment 180. The system of any one of the enumerated embodiments, wherein the one or more processors being configured to identify the subject comprises using a machine learning algorithm to determine a 3D facial surface map of the subject with the camera.
Enumerated embodiment 181. The system of any one of the enumerated embodiments, wherein the one or more processors are further configured to collect data corresponding to facial features of the subject.
Enumerated embodiment 182. The system of any one of the enumerated embodiments, wherein the one or more processors are further configured to calculate an angle of the nose, an angle of the head, or position of a nasal cavity using at least a portion of the data points associated with facial features of the subject.
Enumerated embodiment 183. The system of any one of the enumerated embodiments, wherein the one or more processors being configured to identify the subject comprises the one or more processors being configured to use a machine learning algorithm to determine a 3D facial surface of the subject with the camera without using a depth sensor.
Enumerated embodiment 184. The system of any one of the enumerated embodiments, wherein the one or more processors being configured to identify the subject comprises the one or more processors being configured to compare detected facial features of the subject to a known or verified image of the subject to identify the subject with at least about 70%, at least about 75%, at least about 80%, at least about 85%, at least about 90%, or at least about 95% accuracy.
Enumerated embodiment 185. The system of any one of the enumerated embodiments, further comprising tracking a position of the nose or face of the subject relative to the camera, wherein tracking the position of the nose or face of the subject comprises extrapolating an angle of the nose based upon the at least two points one or more points corresponding to a nose bridge and a tip of a nose of the subject.
Enumerated embodiment 186. The system of any one of the enumerated embodiments, wherein the one or more processors being configured to identify the subject using one or more subject indicia comprises the one or more processors being configured to apply a facial recognition algorithm to identify the subject.
Enumerated embodiment 187. The system of any one of the enumerated embodiments, wherein the one or more subject indicia comprises one or more facial features of the subject.
Enumerated embodiment 188. The system of any one of the enumerated embodiments, wherein the one or more processors being configured to identify the subject comprises the one or more processors being configured to match the one or more facial features of the subject to a data file comprising data corresponding to one or more facial features of the subject.
Enumerated embodiment 189. The system of any one of the enumerated embodiments, wherein the one or more processors being configured to identify the subject comprises the one or more processors being configured to match the one or more facial features of the subject to a data file comprising a verified image of the subject.
Enumerated embodiment 190. The system of any one of the enumerated embodiments, wherein the machine learning algorithm isolates a face within an individual frame of a captured video from the camera for analysis.
Enumerated embodiment 191. The system of any one of the enumerated embodiments, wherein the one or more processors being configured to identify the subject using one or more subject indicia comprises the one or more processors being configured to apply a facial recognition algorithm to identify the subject by matching the one or more subject indicia to a known facial scan of the subject.
Enumerated embodiment 192. The system of any one of the enumerated embodiments, wherein the one or more processors being configured to identify the subject comprises the one or more processors being configured to provide instruction to position a face of the subject within a target view field of the camera.
Enumerated embodiment 193. The system of any one of the enumerated embodiments, wherein the one or more processors being configured to identify the subject comprises the one or more processors being configured to indicate to the subject when the face of the subject is correctly positioned within the target view field of the camera.
Enumerated embodiment 194. The system of any one of the enumerated embodiments, wherein the one or more processors are further configured to continuously identify the subject during performance of the claimed method, or continuously identify the subject at multiple time points during performance of the claimed method.
Enumerated embodiment 195. The system of any one of the enumerated embodiments, wherein the one or more processors are further configured to continuously identify the subject during the actuating of the device, verifying actuation of the device, the determining the alignment state of the device, or any combination thereof.
Enumerated embodiment 196. The system of any one of the enumerated embodiments, wherein the one or more processors are further configured to re-identify the subject if the subject is determined to leave the field of view of the camera during performance of the method.
Enumerated embodiment 196. The system of any one of the enumerated embodiments, wherein the one or more processors are further configured to identify the device by scanning and identify indicia located on the device with the computing device.
Enumerated embodiment 198. The system of any one of the enumerated embodiments, wherein the one or more processors are further configured to verify the device corresponds to a prescription by referencing matching the device to a data file comprising data corresponding to a prescription.
Enumerated embodiment 199. The system of any one of the enumerated embodiments, wherein the one or more processors are further configured to verify the device corresponds to a prescription belonging to the subject based upon a positive device identification, a positive prescription identification, a positive subject identification, or combinations thereof.
Enumerated embodiment 200. The system of any one of the enumerated embodiments, wherein the one or more processors are further configured to identify an impediment to identifying the subject or verifying the device, and prompting the subject to correct the impediment.
Enumerated embodiment 201. The system of any one of the enumerated embodiments, wherein the impediment comprises the subject or the device being positioned too far away from the camera, an improper lighting state, a physical obstruction in view of the camera, an improper subject head angle, a subject position out of view of the camera, or any combination thereof.
Enumerated embodiment 202. The system of any one of the enumerated embodiments, wherein the one or more processors are further configured to continuously identify a position of the device during performance of the claimed method, or continuously identify a position of the device at multiple time points.
Enumerated embodiment 203. The system of any one of the enumerated embodiments, wherein the one or more processors are further configured to continuously identify a position of the device during the actuating of the device, verifying actuation of the device, the determining the alignment state of the device, or any combination thereof.
Enumerated embodiment 204. The system of any one of the enumerated embodiments, wherein the one or more processors are further configured to verify a configuration state of the device based upon whether the housing is visible.
Enumerated embodiment 205. The system of any one of the enumerated embodiments, wherein the one or more processors are further configured to providing feedback to actuate the device.
Enumerated embodiment 206. The system of any one of the enumerated embodiments, wherein the first indicator prompts the subject to actuate the device.
Enumerated embodiment 207. The system of any one of the enumerated embodiments, wherein the third indicator prompts the subject to re-actuate the device.
Enumerated embodiment 208. The system of any one of the enumerated embodiments, wherein the device comprises a securing mechanism preventing actuation of the device.
Enumerated embodiment 209. The system of any one of the enumerated embodiments, wherein the device is in communication with the computing device.
Enumerated embodiment 210. The system of any one of the enumerated embodiments, wherein the securing mechanism is released upon verifying a positive subject identification, a positive prescription identification, a proper alignment state of the device, or combinations thereof.
Enumerated embodiment 211. The system of any one of the enumerated embodiments, wherein the securing mechanism is released upon a signal transmitted from the computing device to the device.
Enumerated embodiment 212. The system of any one of the enumerated embodiments, wherein the recommendation comprises one or more prompts.
Enumerated embodiment 213. The system of any one of the enumerated embodiments, wherein the one or more processors being configured to verify delivery of the composition to the subject comprises the one or more processors being configured to maintain a continuous positive subject identification throughout the actuating the device.
Enumerated embodiment 214. The system of any one of the enumerated embodiments, wherein the one or more processors are further configured to identify a negative subject identification and a positive prescription identification, and not releasing the securing mechanism, preventing actuation of the device.
Enumerated embodiment 215. The system of any one of the enumerated embodiments, wherein the one or more processors are further configured to identify a negative subject identification and a positive prescription identification, and generating a report.
Enumerated embodiment 216. The system of any one of the enumerated embodiments, wherein the data file comprising data corresponding to one or more facial features of the subject, the data file comprising a verified image of the subject, the data file comprising data corresponding to a prescription are stored locally on the computing device.
Enumerated embodiment 217. The system of any one of the enumerated embodiments, wherein the data file comprising data corresponding to one or more facial features of the subject, the data file comprising a verified image of the subject, the data file comprising data corresponding to a prescription are stored remotely from the computing device.
Enumerated embodiment 218. The system of any one of the enumerated embodiments, wherein the one or more processors are further configured to capture one or more images of the subject when the device is actuated.
Enumerated embodiment 219. The system of any one of the enumerated embodiments, wherein the one or more processors are further configured to capture one or more images of the subject during the identifying the subject.
Enumerated embodiment 220. The system of any one of the enumerated embodiments, wherein the marker comprises a bar code, a QR code, or other camera readable code comprising drug data, prescription data, or device identification data.
Enumerated embodiment 221. The system of any one of the enumerated embodiments, wherein the marker comprises an ArUco marker.
Enumerated embodiment 222. The system of any one of the enumerated embodiments, wherein the one or more processors are further configured to update the machine learning algorithm based upon a captured image or video file of the subject.
Enumerated embodiment 223. The system of any one of the enumerated embodiments, wherein the one or more processors are further configured to detect an object or obstruction within a reference frame.
Enumerated embodiment 224. The system of any one of the enumerated embodiments, wherein the one or more processors are further configured to identify objects or obstructions within the reference frame based upon a shape, a contour, a color, or a texture of the object or obstruction.
Enumerated embodiment 225. The system of any one of the enumerated embodiments, wherein the one or more processors are further configured to localize a position of the object or obstruction within a reference frame.
Enumerated embodiment 226. The system of any one of the enumerated embodiments, wherein the one or more processors are further configured to measure a distance between the camera and the subject or device using a relative scale, wherein the relative scale increases the accuracy of the measurement.
Enumerated embodiment 227. The system of any one of the enumerated embodiments, wherein patient identification, detection of device positioning, detection of device actuation, and/or corrective guidance are performed simultaneously.
Enumerated embodiment 228. The system of any one of the enumerated embodiments, wherein the one or more processors are further configured to generate a data report comprising subject identity, device serial number, drug identity, date, time, location, a video of performance of the claimed method, a photo of performance of the claimed system, an actuation event, face position at actuation, device position at actuation, an actuation confirmation, incorrect dose alert, tampering alert or any combination thereof.
Enumerated embodiment 229. The system of any one of the enumerated embodiments, wherein the one or more processors are further configured to store data in a database following performance of any step of the method.
Enumerated embodiment 230. The system of any one of the enumerated embodiments, wherein the data comprises image or video data.
Enumerated embodiment 231. The system of any one of the enumerated embodiments, wherein the image or video data is stored as a JSON object structure.
Enumerated embodiment 232. The system of any one of the enumerated embodiments, wherein the JSON object structure comprises keys mapping the positioning state of the device, the identifying the subject, the actuating the device, or any combination thereof relative to a state of performance of the method.
Enumerated embodiment 233. The system of any one of the enumerated embodiments, wherein the composition comprises a medicament, vaccine, biologic, small molecule, cell line, or therapeutic.
Enumerated embodiment 234. A non-transitory computer-readable medium comprising executable instructions that, when executed by one or more processors, cause the one or more processors to perform the method according to any one of the enumerated embodiments.
Enumerated embodiment 235. A device for delivering a composition, comprising:
a housing transitionable from a first configuration state to a second configuration state, wherein transitioning the housing from the first configuration state to the second configuration state actuates the device;
a marker readable by an electronic device, the marker being exposed in the first configuration state, the marker being at least partially obscured in the second configuration state, the marker indicating the configuration state of the device; and one or more dispensing elements for delivery of the composition upon actuation of the device.
Enumerated embodiment 236. The device of any one of the enumerated embodiments, further comprising a compartment for holding a composition, wherein the composition is delivered upon actuation of the device.
Enumerated embodiment 237. The device of any one of the enumerated embodiments, further comprising an actuation mechanism, wherein,
the device is configured to move between a plurality of configuration states comprising the first configuration state,
the marker is not concealed in the first configuration state,
the device being in the first configuration state indicates that the device has not been actuated; and
the device is configured to verify actuation of the device based on a change in configuration state of the device and obscuring of the marker.
Enumerated embodiment 238. The device of any one of the enumerated embodiments, wherein the marker is a fiducial marker.
Enumerated embodiment 239. The device of any one of the enumerated embodiments, wherein the marker indicates an alignment of the device with respect to a target region of the subject.
Enumerated embodiment 240. The device of any one of the enumerated embodiments, wherein the device is a nasal administration device, wherein the device delivers the composition to the nasal cavity of the subject, or an olfactory region of the subject.
Enumerated embodiment 241. The device of any one of the enumerated embodiments, wherein the housing further comprises a first housing marker which further indicates a configuration state or an actuation state of the device.
Enumerated embodiment 242. The device of any one of the enumerated embodiments, wherein the marker is at least partially obscured by the housing and the first housing marker is visible in the second configuration state of the plurality of configuration states.
Enumerated embodiment 243. The device of any one of the enumerated embodiments, wherein the marker is at least partially concealed by the housing and the first housing marker is not visible in a third configuration state of the plurality of configuration states.
Enumerated embodiment 244. The device of any one of the enumerated embodiments, wherein the marker is a QR code.
Enumerated embodiment 245. The device of any one of the enumerated embodiments, wherein the marker is an ArUco marker.
Enumerated embodiment 246. The device of any one of the enumerated embodiments, wherein the device being in the first configuration state indicates the device can be actuated or is unactuated.
Enumerated embodiment 247. The device of any one of the enumerated embodiments, wherein the marker comprises one or more submarkers.
Enumerated embodiment 248. The device of any one of the enumerated embodiments, wherein, in the second configuration state, a first submarker of the one or more submarkers is concealed by the housing and a second submarker of the one or more submarkers is or is not concealed by the housing and the housing marker is not concealed.
Enumerated embodiment 249. The device of any one of the enumerated embodiments, wherein, in the first configuration state, the first submarker of the one or more submarkers is not concealed, and the second submarker of the one or more submarkers is not concealed, and the first housing marker is not concealed.
Enumerated embodiment 250. The device of any one of the enumerated embodiments, wherein, in the third configuration state, the first submarker of the one or more submarkers is concealed, the second submarker of the one or more submarkers is or is not concealed, and the first housing marker is concealed.
Enumerated embodiment 251. The device of any one of the enumerated embodiments, wherein the device is configured to transition from the first configuration state to the second configuration state when the device is partially inserted into a nasal cavity of the subject, or when pressure is applied to the device by the subject's columella.
Enumerated embodiment 252. The device of any one of the enumerated embodiments, wherein the device is configured to transition from the first configuration state to the second configuration state when the device is partially inserted into the nasal cavity of the subject at a prescribed depth and angle, or when pressure is applied to the device by the subject's columella.
Enumerated embodiment 253. The device of any one of the enumerated embodiments, wherein the marker comprises at least two submarkers which indicate a configuration state of the device.
Enumerated embodiment 254. The device of any one of the enumerated embodiments, a position of the marker indicates a configuration state of the device.
Enumerated embodiment 255. The device of any one of the enumerated embodiments, wherein the position of the marker relative to one or more subject facial features, one or more subject indicia, or combinations thereof indicates an alignment state of the device.
Enumerated embodiment 256. The device of any one of the enumerated embodiments, wherein a pitch or a yaw of the device is calculated based upon a measurement of the marker to a projection of a plane defined by the measurement of the marker.
Enumerated embodiment 257. The device of any one of the enumerated embodiments, wherein the pitch of the device is calculated based upon the projection of the plane using a formula:

$${cos}^{- 1} ( \frac{observed height}{actual height} ) * \frac{180}{π} ,$$

wherein observed height is an observed height of the fiducial marker, and actual height is an actual height of the fiducial marker.
Enumerated embodiment 258. The device of any one of the enumerated embodiments, wherein the pitch of the device is calculated with an accuracy of +/−2°, +/−3°, +/−4°, +/−5°, +/−6°, +/−7°, +/−8°, +/−9°, +/−10°, +/−11°, +/−12°, +/−13°, +/−14°, or +/−15° when the pitch is greater than 20°, or ±10° when the pitch is or is lower than 20°.
Enumerated embodiment 259. The device of any one of the enumerated embodiments, wherein the yaw of the device is calculated based upon a projection onto the plane using a formula:

$${tan}^{- 1} ( \frac{{x}_{1} - {x}_{2}}{{y}_{1} - {y}_{2}} ) * \frac{180}{π} ,$$

wherein x₁ is a horizontal coordinate at a top corner of the marker, x₂ is a horizontal coordinate at a bottom corner of the marker, y₁ is a vertical coordinate at the top corner of the marker, and y₂ is a vertical coordinate at the bottom corner of the marker.
Enumerated embodiment 260. The device of any one of the enumerated embodiments, wherein the accuracy of the yaw relative to the subject's nose is calculated based on an accuracy of a yaw of the device and an accuracy of a yaw of the nose.
Enumerated embodiment 261. The device of any one of the enumerated embodiments, wherein the device comprises a securing mechanism preventing actuation of the device.
Enumerated embodiment 262. The device of any one of the enumerated embodiments, wherein the marker comprises a bar code, a QR code, or other camera readable code comprising drug data, prescription data, or device identification data.
Enumerated embodiment 263. The device of any one of the enumerated embodiments, wherein the marker comprises an ArUco marker.
Enumerated embodiment 264. The device of any one of the enumerated embodiments, wherein the device is in communication with a computing device.
Enumerated embodiment 265. The device of any one of the enumerated embodiments, wherein, the device is configured to receive a first indicator, wherein the device allows actuation of the device upon receiving the first indicator.
Enumerated embodiment 266. The device of any one of the enumerated embodiments, wherein the device is configured to receive the first indicator when the device in the first configuration state.
Enumerated embodiment 267. The device of any one of the enumerated embodiments, wherein, the device is configured to receive a second indicator after receiving the first indicator, wherein the device is configured to prevent actuation upon receiving the second indicator.
Enumerated embodiment 268. The device of any one of the enumerated embodiments, wherein the device is configured to receive the second indicator when the device is not in the first configuration state.
Enumerated embodiment 269. The device of any one of the enumerated embodiments, wherein the device is configured to receive a third indicator after receiving the second indicator, wherein the device allows actuation of the device upon receiving the third indicator.
Enumerated embodiment 270. The device of any one of the enumerated embodiments, wherein the device is configured to receive the third indicator when the device is in the first configuration state.
Enumerated embodiment 271. The device of any one of the enumerated embodiments, wherein the composition comprises a medicament, vaccine, biologic, small molecule, cell line, or therapeutic.
Enumerated embodiment 272. The device of any one of the enumerated embodiments, wherein the first housing marker is a feature of the housing.
Enumerated embodiment 273. The device of any one of the enumerated embodiments, wherein the first housing marker comprises an annular ring.
Enumerated embodiment 274. The device of any one of the enumerated embodiments, wherein the first housing marker comprises a color distinct from the housing.
Enumerated embodiment 275. The device of any one of the enumerated embodiments, wherein the marker is visible, the first housing marker in not visible in a fourth configuration state of the plurality of configuration states, wherein the fourth configuration state confirms actuation of the device or that the device does not comprise the composition.
Enumerated embodiment 276. The device of any one of the enumerated embodiments, further comprising a second housing transitionable from the second configuration state to the third configuration state.
Enumerated embodiment 277. The device of any one of the enumerated embodiments, wherein the second housing comprises a second housing marker.
Enumerated embodiment 278. The device of any one of the enumerated embodiments, wherein second housing marker is not visible in the first configuration state and the second configuration state of the plurality of configuration states.
Enumerated embodiment 279. The device of any one of the enumerated embodiments, wherein second housing marker is visible in the third configuration state of the plurality of configuration states.
Enumerated embodiment 280. The device of any one of the enumerated embodiments, wherein the maker is visible, the first housing marker in not visible, and the second housing marker is visible in the fourth configuration state of the plurality of configuration states, wherein the fourth configuration state confirms actuation of the device or that the device does not comprise the composition.
Enumerated embodiment 281. The method of any one of the enumerated embodiments, wherein the housing further comprises a first housing marker.
Enumerated embodiment 282. The method of any one of the enumerated embodiments, wherein the first housing marker is a feature of the housing.
Enumerated embodiment 283. The method of any one of the enumerated embodiments, wherein first housing marker comprises an annular ring.
Enumerated embodiment 284. The method of any one of the enumerated embodiments, wherein first housing marker comprises a color distinct from the housing.
Enumerated embodiment 285. The method of any one of the enumerated embodiments, wherein the second housing further comprises a second housing marker.
Enumerated embodiment 286. The system of any one of the enumerated embodiments, wherein the housing further comprises a first housing marker.
Enumerated embodiment 287. The system of any one of the enumerated embodiments, wherein the first housing marker is a feature of the housing.
Enumerated embodiment 288. The system of any one of the enumerated embodiments, wherein first housing marker comprises an annular ring.
Enumerated embodiment 289. The system of any one of the enumerated embodiments, wherein first housing marker comprises a color distinct from the housing.
Enumerated embodiment 290. The system of any one of the enumerated embodiments, wherein the second housing further comprises a second housing marker.

All publications, patents, and patent applications mentioned in this specification are herein incorporated by reference to the same extent as if each individual publication, patent, or patent application was specifically and individually indicated to be incorporated by reference.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A device for delivering a composition, comprising:
one or more housings;
a marker readable by an electronic device; and
one or more dispensing elements for delivery of the composition upon actuation of the device,
wherein the device is transitionable between a plurality of configuration states based on a position of the marker and the one or more housings, and
wherein the marker (i) is exposed in a first configuration state, and (ii) is at least partially obscured in a second configuration state.

2. The device of claim 1, wherein the device is configured to transition from the first configuration state to the second configuration state when pressure is applied to the device by the subject's columella.

3. The device of claim 1, wherein the device is configured to receive a first indicator when the device is in the first configuration state, wherein the device allows actuation of the device upon receiving the first indicator.

4. The device of claim 1, wherein the device is configured to verify actuation of the device based on a change in configuration state of the device and obscuring of the marker.

5. The device of claim 1, wherein the one or more housings comprises a first housing comprising a first housing marker which further indicates a configuration state or an actuation state of the device, wherein the first housing marker is visible in the second configuration state of the plurality of configuration states.

6. The device of claim 5, wherein the one or more housings further comprises a second housing transitionable from the second configuration state to a third configuration state.

7. The device of claim 6, wherein the second housing comprises a second housing marker, wherein the second housing marker is not visible in the first configuration state and the second configuration state of the plurality of configuration states, and wherein the second housing marker is visible in the third configuration state of the plurality of configuration states.

8. The device of claim 6, wherein the marker is visible, the first housing marker is not visible, and the second housing marker is visible in a fourth configuration state of the plurality of configuration states, wherein the fourth configuration state confirms actuation of the device or that the device does not comprise the composition.

9. The device of claim 1, wherein the marker is a fiducial marker.

10. The device of claim 1, wherein the device is a nasal administration device configured for delivering the composition to a nasal cavity of the subject.

11. A system, comprising:
one or more processors; and
a memory comprising executable instructions which, when executed by the one or more processors, cause the system to:
capture one or more images of a subject;
identify the subject based on the one or more images of the subject and one or more criteria, wherein the one or more criteria comprises the subject being in need of a composition;
capture one or more images of a device for delivering the composition to a target region of the subject, wherein the device comprises a marker and one or more housings, wherein the device is configured to transition between a plurality of configuration states based on a position of the marker and the one or more housings;
identify the device as being in a first configuration state of the plurality of configuration states based on the one or more images of the device, wherein the marker is not concealed by the one or more housings in the first configuration state; and
at least one of:
i. provide an indicator to deliver the composition to the target region based on identifying the device as being in the first configuration state and identifying the subject based on the one or more criteria;
ii. display a recommendation to deliver the composition to the target region based on identifying the device as being in the proper configuration state and identifying the subject based on the one or more criteria; and
iii. verify actuation of the device based on a change in the configuration state of the device, thereby verifying delivery of the composition of the subject.

12. The system of claim 11, wherein the target region is in a nasal cavity of the subject.

13. The system of claim 11, wherein the marker is a fiducial marker.

14. The system of claim 11, wherein the one or more processors are further configured to determine an alignment state of the device and the subject based upon a position of the marker relative to one or more subject facial features, the one or more criteria, or combinations thereof.

15. The system of claim 14, wherein the position comprises the alignment state of the device, and wherein the one or more processors are further configured to provide a recommendation to adjust the alignment state of the device if it is determined the alignment state is improper relative to the one or more subject facial features.

16. The system of claim 11, wherein the one or more processors are further configured to calculate an angle of the nose, an angle of the head, or position of a nasal cavity using at least a portion of the data points associated with facial features of the subject.

17. The system of claim 11, wherein the one or more processors are configured to analyze an image amongst the one or more images, wherein the image is of the subject's face, and associating at least 68 data points to facial features of the subject, wherein the data points associated with facial features of the subject comprise at least two points corresponding to a nose bridge and a tip of a nose of the subject

18. The system of claim 11, wherein the one or more processors are further configured to verify the device corresponds to a prescription belonging to the subject based upon a positive device identification, a positive prescription identification, a positive subject identification, or combinations thereof.

19. The system of claim 11, wherein the one or more processors are further configured to provide a second indicator, wherein the second indicator prevents the device from delivering the composition.

20. The system of claim 19, wherein the second indicator is provided based on the subject not being identifiable or the device not being in the first configuration state.
